(19) United States
(12) Patent Application Publication
(10) Pub. No.: US 2011/0038850 A1
(43) Pub. Date: Feb. 17, 2011

(54) G Protein-Coupled Receptor and Modulators Thereof For The Treatment of Gaba-Related Neurological Disorders Including Sleep-Related Disorders

(76) Inventors: Didier Bagnol, San Diego, CA (US); Andrew J. Grottick, Chula Vista, CA (US)

Correspondence Address: Arena Pharmaceuticals, Inc.;Bozicevic, Field & Francis LLP, 1900 University Avenue, Suite 200, East Palo Alto, CA, 94303, US

(21) Appl. No.: 12/521,274

(22) PCT Filed: Jan. 4, 2008
(86) PCT No.: PCT/US2008/000116

§ 371 Date: May 12, 2010

Related U.S. Application Data
(60) Provisional application No. 60/878,796, filed on Jan. 5, 2007.

(51) Int. Cl. A61K 38/16; A61K 38/02; A61P 25/00; A61K 39/395; A61P 25/22; A61P 25/18; A61P 25/06; A61P 25/08; A61P 25/24; A61P 25/28; G01N 33/567

(52) U.S. Cl. 424/130.1; 514/17.7; 435/7.21

(57) ABSTRACT

The present invention relates to methods of using BRS-3 to screen candidate compounds as compounds suitable for the treatment of sleep-related disorders. Inverse agonists and antagonists of the invention are useful as therapeutic agents for promoting sleep and for preventing or treating sleep disorders ameliorated by promoting sleep, such as insomnia and the like. Agonists and partial agonists of the invention are useful as therapeutic agents for promoting wakefulness and for preventing or treating excessive sleepiness, such as excessive sleepiness associated with narcolepsy and the like. The invention further relates to methods of using a BRS-3 to screen candidate compounds as pharmaceutical agents for a GABA-related neurological disorder such as a sleep disorder, an anxiety disorder, a convulsive disorder, migraine, a depressive disorder, a psychotic disorder, or a cognitive disorder. Compounds of the invention encompass compounds having sleep-promoting, wakefulness-promoting, anxiolytic, anticonvulsant, antidepressant, antipsychotic, and cognition-enhancing activities.

### FIELD OF THE INVENTION

The present invention relates to methods of using a G protein-coupled receptor (GPCR) to screen candidate compounds as compounds suitable for the treatment of sleep-related disorders. Inverse agonists and antagonists of the invention are useful as therapeutic agents for promoting sleep and for preventing or treating sleep disorders ameliorated by promoting sleep, such as insomnia and the like. Agonists and partial agonists of the invention are useful as therapeutic agents for promoting wakefulness and for preventing or treating excessive sleepiness, such as excessive sleepiness associated with narcolepsy and the like. The invention further relates to methods of using a GPCR to screen candidate compounds as pharmaceutical agents for a GABA-related neurological disorder such as a sleep disorder, an anxiety disorder, a convulsive disorder, migraine, a depressive disorder, a psychotic disorder, or a cognitive disorder. Compounds of the invention encompass compounds having sleep-promoting, wakefulness-promoting, anxiolytic, anticonvulsant, antidepressant, antipsychotic, and cognition-enhancing activities.

### BACKGROUND OF THE INVENTION

The following discussion is intended to facilitate the understanding of the invention, but is not intended nor admitted to be prior art to the invention.

A. Sleep Disorders

Sleep is part of a basic physiologic rhythym that is characterized in humans by three states: wakefulness, non-rapid eye movement (NREM) sleep, and rapid eye movement (REM) sleep. Sleep disorders are disturbances of usual sleep patterns or behaviors.

The term “insomnia” refers to the perception of inadequate or non-restful sleep by a subject. Problems can occur, for example, with one or more of the following: sleep onset, sleep maintenance or early morning awakenings. Insomnia is a frequent complaint, reported by 32% of the adult population surveyed in the Los Angeles area (Bixler et al, Am J Psychiatry (1979) 136:1257-1262). Fully 45% of the surveyed population of Alachua County, Florida, reported trouble getting to sleep or staying asleep (Karacan et al, Soc Sci Med (1976) 10:239-244).

Excessive daytime sleepiness (hypersomnia) is a cardinal feature of narcolepsy. Hypersomnia is also associated with a number of neurological disorders (multiple sclerosis, myonic dystrophy, Parkinson's disease), psychiatric disorders (depression, schizophrenia) and other disorders (obstructive sleep apnea, night-shift sleep disorder, drug-induced sedation).

Sleep disorders are common and can lead to significant disability and social and financial costs (for example, road traffic accidents, poor work performance). See, e.g., Szabadi, Br J Clin Pharmacol (2006) 61:761-766; Harris, Respir Care Clin (2005) 11:567-586; American Academy of Sleep Medicine, ICSD—International classification of sleep disorders, revised: Diagnostic and coding manual, American Academy of Sleep Medicine, 2001.

B. Bombesin Receptor Subtype-3 (BRS-3)

Bombesin is a 14 amino acid peptide isolated from frog skin. Bombesin Receptor Subtype-3 BRS-3 G protein-coupled receptor (BRS-3; e.g., human BRS-3, GenBank® Accession No. AAA35604 and alleles thereof; e.g., mouse BRS-3, GenBank® Accession No. NP_{—}033896 and alleles thereof; e.g., rat BRS-3, GenBank® Accession No. AF510984 and alleles thereof) exhibits about 50% homology to gastric-releasing peptide receptor (GRP-R; e.g., human GRP-R, GenBank® Accession No. NP_{—}005305) and neuromedin B receptor (NMB-R; e.g., human NMB-R, GenBank® Accession No. NP_{—}002502), and together they form the bombesin-like receptor group. BRS-3 is selectively expressed in tissues including hypothalamus and uterus. BRS-3 activation leads to increased accumulation of intracellular inositol 1,4,5-triphosphate (IP3), consistent with BRS-3 being coupled to Gq. In recent studies, BRS-3 knockout mice developed obesity, diabetes, and hypertension [Ohki-Hamazaki et al., Nature (1997) 390:165-169].

C. Gamma-Aminobutyric Acid (GABA)

GABA (gamma-aminobutyric acid) is the major inhibitory transmitter in the brain. Glutamic acid decarboxylase 67 (GAD67) is a marker for GABAergic neurons. The gamma-aminobutyric acid type A (GABA_{A}) receptors are the major inhibitory neuronal receptors in the mammalian brain. Their activation by GABA opens the intrinsic ion channel, enabling chloride flux into the cell with subsequent hyperpolarization. The GABA_{A} receptors are pentameric structures, most commonly composed of α, β and γ subunits with a stoichiometry of two α subunits, two β subunits and one γ subunit. In mammals, several GABA_{A} receptor subunit isoforms have been cloned, including α1-6, β1-3 and γ1-3. Subunit composition of a GABA_{A} receptor determines its pharmacological properties. Benzodiazepines, e.g. diazepam, and the like produce their therapeutic effects by binding to a specific site on the GABA_{A} receptor and allosterically enhancing the GABA-evoked chloride flux. Other compounds that bind to the benzodiazepine binding site, e.g. FG 7142 (N-methyl-β-carboline-3-carboxamide) and α5IA (3-(5-methylisoxazol-3-yl)-6-[(1-methyl-1,2,3-triazol-4-yl)methyl-oxy]-1,2,4-triazolo[3,4-α]phthalazine), can allosterically reduce the GABA-evoked chloride flux.

GABA plays a role in regulating sleep, anxiety, convulsion, depression, psychosis, cognition, and the like, states in mammals.

(Da Settimo et al., Curr Med Chem (2007) 14:2680-2701; Mohler, Cell Tissue Res (2006) 326:505-516; Crestani et al., Mol Pharmacol (2001) 59:442-445; Nutt, J Clin Sleep Med (2006) 15:S7-S11; Lippa et al, Proc Natl Acad Sci USA (2005) 102:7380-7385; McKernan et al, Nat Neurosci (2000) 3:587-92; Low et al, Science (2000) 290:131-134; Crestani et al, Proc Natl Acad Sci USA (2002) 99:8980-8985; Dawson et al, J Pharmacol Exp Ther (2006) 316:1335-1345; Collinson et al, J Neurosci (2002) 22:5572-5580; Calabresi et al, Trends Pharmacol Sci (2007) 28:188-195; Sanacora et al, CNS Neurol Disord Drug Targets (2007) 6:127-140.)

D. Hypothalamus

The dorsomedial hypothalamic nucleus (DMH) is one the largest sources of input to the ventrolateral preoptic nucleus (VLPO) and the lateral hypothalamus area (LH), respectively involved in the promotion of sleep and wakefulness (Chou et al., J Neurosci (2002) 22:977-990; Chou et al., J Neurosci (2003) 23:10691-10702; Saper et al., Nature (2005) 437:1257-1263; Thompson et al., J Comp Neurol (1996) 376:143-173; Yoshida et al., J Comp Neurol (2006) 494:845-861). Particularly, DMH neurons contain the principal inhibitory neurotransmitter gamma-aminobutyric acid (GABA) projecting to the sleep-promoting nucleus VLPO while glutamate-thyrotropin-releasing hormone neurons project to the wake-promoting LH including orexin neurons (Chou et al., J Neurosci (2003) 23:10691-10702).

VLPO GABAergic neurons control directly the excitability of wake-promoting orexin neurons in the LH (Gong et al., J Physiol (2004) 556:935-946). Orexin neurons in the LH play a critical role in promoting wakefulness in mouse, dog and human (Chemelli et al., Cell (1999) 98:437-451; Hungs et al., Genome Res (2001) 11:531-539; Lin et al., Cell (1999) 98:365-376; Peyron et al., Nat Med (2000) 6:991-997).

E. G Protein-Coupled Receptors

Although a number of receptor classes exist in humans, by far the most abundant and therapeutically relevant is represented by the G protein-coupled receptor (GPCR) class. It is estimated that there are some 30,000-40,000 genes within the human genome, and of these, approximately 2% are estimated to code for GPCRs.

GPCRs represent an important area for the development of pharmaceutical products: from approximately 20 of the 100 known GPCRs, approximately 60% of all prescription pharmaceuticals have been developed. For example, in 1999, of the top 100 brand name prescription drugs, the following drugs interact with GPCRs (the primary diseases and/or disorders treated related to the drug is indicated in parentheses):

| | | |
|---|---|---|
| Claritin ® (allergies) | Prozac ® (depression) | Vasotec ® (hypertension) |
| Paxil ® (depression) | Zoloft ® (depression) | Zyprexa ®(psychotic disorder) |
| Cozaar ® (hypertension) | Imitrex ® (migraine) | Zantac ® (reflux) |
| Propulsid ® (reflux disease) | Risperdal ® (schizophrenia) | Serevent ® (asthma) |
| Pepcid ® (reflux) | Gaster ® (ulcers) | Atrovent ® (bronchospasm) |
| Effexor ® (depression) | Depakote ® (epilepsy) | Cardura ®(prostatic hypertrophy) |
| Allegra ® (allergies) | Lupron ® (prostate cancer) | Zoladex ® (prostate cancer) |
| Diprivan ® (anesthesia) | BuSpar ® (anxiety) | Ventolin ® (bronchospasm) |
| Hytrin ® (hypertension) | Wellbutrin ® (depression) | Zyrtec ® (rhinitis) |
| Plavix ® (MI/stroke) | Toprol-XL ® (hypertension) | Tenormin ® (angina) |
| Xalatan ® (glaucoma) | Singulair ® (asthma) | Diovan ® (hypertension) |
| Harnal ® (prostatic hyperplasia) | | |
| (Med Ad News 1999 Data). | | |

GPCRs share a common structural motif, having seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when a ligand binds with the receptor (often referred to as “activation” of the receptor), there is a change in the conformation of the receptor that facilitates coupling between the intracellular region and an intracellular “G-protein.” It has been reported that GPCRs are “promiscuous” with respect to G proteins, i.e., that a GPCR can interact with more than one G protein. See, Kenakin, Life Sciences (1988) 43:1095-1101. Although other G proteins exist, currently, Gq, Gs, Gi, Gz and Go are G proteins that have been identified. Ligand-activated GPCR coupling with the G-protein initiates a signaling cascade process (referred to as “signal transduction”). Under normal conditions, signal transduction ultimately results in cellular activation or cellular inhibition. Although not wishing to be bound to theory, it is thought that the IC-3 loop as well as the carboxy terminus of the receptor interact with the G protein.

Gs-coupled GPCRs increase intracellular cAMP levels. GPCRs coupled to Gi, Go, or Gz decrease intracellular cAMP levels. Gq-coupled GPCRs increase intracellular IP3 and Ca²⁺ levels.

There are also promiscuous G proteins, which appear to couple several classes of GPCRs to the phospholipase C pathway, such as G15 or G16 [Offermanns & Simon, J Biol Chem (1995) 270:15175-80], or chimeric G proteins designed to couple a large number of different GPCRs to the same pathway, e.g. phospholipase C [Milligan & Rees, Trends in Pharmaceutical Sciences (1999) 20:118-24]. A GPCR coupled to the phospholipase C pathway increases intracellular IP3 and Ca²⁺ levels.

Under physiological conditions, GPCRs exist in the cell membrane in equilibrium between two different conformations: an “inactive” state and an “active” state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response.

A receptor may be stabilized in an active state by a ligand or a compound such as a drug. Recent discoveries, including but not exclusively limited to modifications to the amino acid sequence of the receptor, provide means other than ligands or drugs to promote and stabilize the receptor in the active state conformation. These means effectively stabilize the receptor in an active state by simulating the effect of a ligand binding to the receptor. Stabilization by such ligand-independent means is termed “constitutive receptor activation.”

### SUMMARY OF THE INVENTION

Nucleotide sequence encoding human BRS-3 polypeptide is given in SEQ ID NO: 1; the amino acid sequence of said encoded human BRS-3 polypeptide is given in SEQ ID NO: 2.

Applicants have unexpectedly discovered that in the dorsomedial hypothalamic nucleus (DMH) the majority of BRS-3 neurons are GABAergic neurons. The present invention features methods relating to BRS-3 for screening candidate compounds as compounds suitable for the treatment of sleep-related disorders. The present invention features methods relating to BRS-3 for screening candidate compounds as pharmaceutical agents for a GABA-related neurological disorder such as a sleep disorder, an anxiety disorder, a convulsive disorder, migraine, a depressive disorder, a psychotic disorder, and a cognitive disorder. Inverse agonists and antagonists of the invention are useful as therapeutic agents for promoting sleep and for the prevention or treatment of disorders ameliorated by promoting sleep including, but not limited to, insomnia and the like. Inverse agonists and antagonists of the invention are useful as therapeutic agents for a sleep disorder ameliorated by promoting sleep such as Insomnia, an anxiety disorder such as Generalized Anxiety Disorder or Panic Attack, a convulsive disorder such as Epilepsy, Migraine, a depressive disorder such as Major Depressive Disorder, and a psychotic disorder such as Schizophrenia. Agonists and partial agonists of the invention are useful as therapeutic agents for promoting wakefulness and for preventing or treating excessive sleepiness, such as excessive sleepiness associated with narcolepsy and the like. Agonists and partial agonists of the invention are useful as therapeutic agents for a sleep disorder ameliorated by promoting wakefulness such as Narcolepsy and a cognitive disorder such as Dementia or Dementia of the Alzheimer's Type.

In a first aspect, the invention features a method for identifying compounds suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder, comprising the steps of

(a) contacting a candidate compound with a host cell or with membrane of a host cell that expresses a GPCR, wherein the GPCR comprises an amino acid sequence selected from the group consisting of:
(i) the amino acid sequence of SEQ ID NO: 2;
(ii) amino acids 2-399 of SEQ ID NO: 2;
(iii) the amino acid sequence of a G protein-coupled receptor encoded by a polynucleotide that is amplifiable by polymerase chain reaction (PCR) on a human DNA sample using specific primers SEQ ID NO: 3 and SEQ ID NO: 4;
(iv) the amino acid sequence of a G protein-coupled receptor encoded by a polynucleotide that hybridizes under stringent conditions to the complement of SEQ ID NO: 1;
(v) the amino acid sequence of a G protein-coupled receptor having an amino acid sequence derived from SEQ ID NO: 2 by substitution, deletion or addition of one or several amino acids in the amino acid sequence of SEQ ID NO: 2;
(vi) the amino acid sequence of a G protein-coupled receptor having at least about 75%, at least about 80%, at least about 85%, at least about 90% or at least about 95% identity to SEQ ID NO: 2;
(vii) the amino acid sequence of a G protein-coupled receptor that is a constitutively active version of a receptor having SEQ ID NO: 2; and
(viii) a biologically active fragment of any one of (i) to (vii); and

(b) determining the ability of the candidate compound to inhibit functionality of the receptor,

wherein the ability of the candidate compound to inhibit functionality of the GPCR is indicative of the candidate compound being a compound suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder.

In certain embodiments, said method is a method for identifying compounds suitable for promoting sleep. In certain embodiments, said method is a method for identifying compounds suitable for preventing or treating GABA-related neurological disorder selected from the group consisting of a sleep disorder ameliorated by promoting sleep, an anxiety disorder, a convulsive disorder, migraine, a depressive disorder, and a psychotic disorder. In certain embodiments, said method is a method for identifying compounds suitable for preventing or treating a sleep disorder ameliorated by promoting sleep. In certain embodiments, said method is a method for identifying compounds suitable for preventing or treating an anxiety disorder. In certain embodiments, said method is a method for identifying compounds suitable for preventing or treating a convulsive disorder. In certain embodiments, said method is a method for identifying compounds suitable for preventing or treating migraine. In certain embodiments, said method is a method for identifying compounds suitable for preventing or treating a depressive disorder. In certain embodiments, said method is a method for identifying compounds suitable for preventing or treating a psychotic disorder.

The invention additionally features a method for identifying compounds suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder, comprising steps (a) and (b) of this first aspect, and further comprising:
(c) optionally synthesizing a compound which inhibits functionality of the GPCR in step (b);
(d) administering a compound which inhibits functionality of the GPCR in step (b) to a mammal; and
(e) determining whether the compound promotes sleep, has anxiolytic activity, has anticonvulsant activity, has anti-migraine activity, has antidepressant activity, or has antipsychotic activity in the mammal;
   wherein the ability of the candidate compound to promote sleep, to show anxiolytic activity, to show anticonvulsant activity, to show anti-migraine activity, to show antidepressant activity, or to show antipsychotic activity in the mammal is indicative of the candidate compound being a compound suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder.

In certain embodiments, said method is a method for identifying compounds suitable for promoting sleep. In certain embodiments, said method is a method for identifying compounds suitable for preventing or treating GABA-related neurological disorder selected from the group consisting of a sleep disorder ameliorated by promoting sleep, an anxiety disorder, a convulsive disorder, migraine, a depressive disorder, and a psychotic disorder. In certain embodiments, said method is a method for identifying compounds suitable for preventing or treating a sleep disorder ameliorated by promoting sleep. In certain embodiments, said method is a method for identifying compounds suitable for preventing or treating an anxiety disorder. In certain embodiments, said method is a method for identifying compounds suitable for preventing or treating a convulsive disorder. In certain embodiments, said method is a method for identifying compounds suitable for preventing or treating migraine. In certain embodiments, said method is a method for identifying compounds suitable for preventing or treating a depressive disorder. In certain embodiments, said method is a method for identifying compounds suitable for preventing or treating a psychotic disorder.

In some embodiments, the mammal is a human. In some embodiments, the mammal is a non-human mammal. In some embodiments, the non-human mammal is a laboratory animal. In some embodiments, the non-human mammal is a non-human primate. In some embodiments, the non-human mammal is a rodent. In some embodiments, the non-human mammal is a rat. In some embodiments, the non-human mammal is a mouse.

In some embodiments, said determining whether the compound promotes sleep in the mammal comprises polysomnography.

In some embodiments, the method comprises identifying an inverse agonist of the GPCR. In some embodiments, said method further comprises formulating the inverse agonist as a pharmaceutical.

In some embodiments, the method comprises identifying an antagonist of the GPCR. In some embodiments, said method further comprises formulating the antagonist as a pharmaceutical.

In some embodiments, said contacting comprises contacting in the presence of a known ligand of the GPCR. In some embodiments, said contacting comprises contacting in the presence of a known ligand of endogenous human BRS-3.

In some embodiments, said contacting comprises contacting in the presence of a known agonist of the GPCR. In some embodiments, the known agonist of the GPCR is a known agonist of endogenous human BRS-3. In some embodiments relating to said contacting comprising contacting in the presence of a known agonist of the GPCR, the candidate compound is contacted with the GPCR prior to the known agonist being contacted with the GPCR. In some embodiments relating to said contacting comprising contacting in the presence of a known agonist of the GPCR, the candidate compound is contacted with the GPCR for a period of up to several minutes prior to the known agonist being contacted with the GPCR. In some embodiments relating to said contacting comprising contacting in the presence of a known agonist of the GPCR, the candidate compound is contacted with the GPCR for a period of up to about 5 min, of up to about 10 min or of up to about 30 min prior to the known agonist being contacted with the GPCR.

In some embodiments, said contacting comprises contacting in the presence of a known agonist of the GPCR, wherein the known agonist of the GPCR is a compound selected from Table D. In some embodiments, said contacting comprises contacting in the presence of a known agonist of the GPCR, wherein the known agonist of the GPCR is Compound D28, Compound D30, Compound D31 or Compound D34.

In some embodiments, said contacting comprises contacting in the absence of a known ligand of the GPCR. In some embodiments, said contacting comprises contacting in the absence of a known ligand of endogenous human BRS-3. In some embodiments, said contacting comprises contacting in the absence of a known agonist of the GPCR. In some embodiments, said contacting comprises contacting in the absence of a known agonist of endogenous human BRS-3.

In some embodiments, the method comprises detecting a second messenger.

In some embodiments, said determining is by a process comprising the measurement of a level of a second messenger selected from the group consisting of cyclic AMP (cAMP), cyclic GMP (cGMP), inositol 1,4,5-triphosphate (IP3), diacylglycerol (DAG), MAP kinase activity, MAPK/ERK kinase kinase-1 (MEKK1) activity, and Ca²⁺. In some embodiments, said second messenger is IP3. In some embodiments, the level of intracellular IP3 is decreased. In some embodiments, said second messenger is Ca²⁺. In some embodiments, the level of intracellular Ca²⁺ is decreased.

In some embodiments, said determining is by a process comprising the use of a Melanophore assay. In some embodiments, the melanophore cells undergo pigment dispersion. In some embodiments, the candidate compound inhibits agonist induced pigment dispersion. In some embodiments, the candidate compound inhibits constitutively (e.g., agonist independent) induced pigment dispersion.

In some embodiments, said determining is by a process comprising the measurement of GTPγS binding to membrane comprising the GPCR. In some embodiments, GTPγS binding to membrane comprising the GPCR is decreased.

In some embodiments, the method further comprises the step of comparing the modulation of the GPCR caused by the candidate compound to a second modulation of the GPCR caused by contacting the GPCR with a known modulator of the GPCR.

In some embodiments, the baseline intracellular response is inhibited in the presence of the candidate compound by at least about 10%, at least about 20%, at least about 30%, at least about 40%, at least about 50%, at least about 60%, at least about 70%, at least about 75%, at least about 80%, at least about 85%, at least about 90%, or at least about 95% as compared with the baseline response in the absence of the candidate compound.

In some embodiments, the baseline intracellular response (e.g., the response in the absence of a known agonist) is inhibited in the presence of the inverse agonist by at least about 10%, at least about 20%, at least about 30%, at least about 40%, at least about 50%, at least about 60%, at least about 70%, at least about 75%, at least about 80%, at least about 85%, at least about 90%, or at least about 95% as compared with the baseline response in the absence of the inverse agonist.

In some embodiments, the baseline intracellular response (e.g., the response in the presence of a known agonist) is inhibited in the presence of the antagonist by at least about 10%, at least about 20%, at least about 30%, at least about 40%, at least about 50%, at least about 60%, at least about 70%, at least about 75%, at least about 80%, at least about 85%, at least about 90%, or at least about 95% as compared with the baseline response in the absence of the antagonist.

The invention also relates to a method for identifying compounds suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder, comprising the steps of:

(a′) contacting a host cell or membrane of a host cell that expresses a GPCR with an optionally labeled known ligand to the GPCR in the presence or absence of a candidate compound, wherein the GPCR comprises an amino acid sequence selected from the group consisting of:
(i) the amino acid sequence of SEQ ID NO: 2;
(ii) amino acids 2-399 of SEQ ID NO: 2;
(iii) the amino acid sequence of a G protein-coupled receptor encoded by a polynucleotide that is amplifiable by polymerase chain reaction (PCR) on a human DNA sample using specific primers SEQ ID NO: 3 and SEQ ID NO: 4;
(iv) the amino acid sequence of a G protein-coupled receptor encoded by a polynucleotide that hybridizes under stringent conditions to the complement of SEQ ID NO: 1;
(v) the amino acid sequence of a G protein-coupled receptor having an amino acid sequence derived from SEQ ID NO: 2 by substitution, deletion or addition of one or several amino acids in the amino acid sequence of SEQ ID NO: 2;
(vi) the amino acid sequence of a G protein-coupled receptor having at least about 75%, at least about 80%, at least about 85%, at least about 90% or at least about 95% identity to SEQ ID NO: 2;
(vii) the amino acid sequence of a G protein-coupled receptor that is a constitutively active version of a receptor having SEQ ID NO: 2; and
(viii) a biologically active fragment of any one of (i) to (vii); and

(b′) detecting the complex between said known ligand and said GPCR;

(c′) determining whether less of said complex is formed in the presence of the candidate compound than in the absence of the candidate compound;

(d′) optionally synthesizing a compound in the presence of which less of said complex is formed in step (c′);

(e′) administering a compound in the presence of which less of said complex is formed in step (c′) to a mammal; and

(f) determining whether the compound promotes sleep, has anxiolytic activity, has anticonvulsant activity, has anti-migraine activity, has antidepressant activity, or has antipsychotic activity in the mammal;

wherein the ability of the candidate compound to promote sleep, to show anxiolytic activity, to show anticonvulsant activity, to show anti-migraine activity, to show antidepressant activity, or to show antipsychotic activity in the mammal is indicative of the candidate compound being a compound suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder.

In certain embodiments, said method is a method for identifying compounds suitable for promoting sleep. In certain embodiments, said method is a method for identifying compounds suitable for preventing or treating GABA-related neurological disorder selected from the group consisting of a sleep disorder ameliorated by promoting sleep, an anxiety disorder, a convulsive disorder, migraine, a depressive disorder, and a psychotic disorder. In certain embodiments, said method is a method for identifying compounds suitable for preventing or treating a sleep disorder ameliorated by promoting sleep. In certain embodiments, said method is a method for identifying compounds suitable for preventing or treating an anxiety disorder. In certain embodiments, said method is a method for identifying compounds suitable for preventing or treating a convulsive disorder. In certain embodiments, said method is a method for identifying compounds suitable for preventing or treating migraine. In certain embodiments, said method is a method for identifying compounds suitable for preventing or treating a depressive disorder. In certain embodiments, said method is a method for identifying compounds suitable for preventing or treating a psychotic disorder.

In some embodiments, the mammal is a human. In some embodiments, the mammal is a non-human mammal. In some embodiments, the non-human mammal is a laboratoryanimal. In some embodiments, the non-human mammal is a non-human primate. In some embodiments, the non-human mammal is a rodent. In some embodiments, the non-human mammal is a rat. In some embodiments, the non-human mammal is a mouse.

In some embodiments, said determining whether the compound promotes sleep in the mammal comprises polysomnography.

In some embodiments, said optionally labeled known ligand is radiolabeled.

In some embodiments, the known ligand is a compound selected from Table D. In some embodiments, the known ligand is Compound D28, Compound D30, Compound D31, or Compound D34. In some embodiments, the known ligand is a compound selected from Table E. In some embodiments, the known ligand is Compound E21 or Compound E22.

In some embodiments, said determining whether less of said complex is formed in the presence in the presence of the candidate compound than in the absence of the candidate compound comprises determining whether at least about 10% less, at least about 20% less, at least about 30% less, at least about 40% less, at least about 50% less, at least about 60% less, at least about 70% less, at least about 75% less, at least about 80% less, at least about 85% less, at least about 90% less, or at least about 95% less of said complex is formed in the presence in the presence of the candidate compound than in the absence of the candidate compound.

In some embodiments, said method is for isolating compounds suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder.

In some embodiments, the sleep disorder comprises fragmented sleep architecture. In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the host cell is a eukaryotic cell.

In some embodiments, the host cell is a mammalian cell. In some embodiments, the mammalian host cell is a CHO cell, a COS-7 cell, an MCB3901 cell, a 293 cell or a 293T cell.

In some embodiments, the host cell is a yeast cell.

In some embodiments, the host cell is a melanophore cell.

In some embodiments, the G protein-coupled receptor comprises the amino acid sequence of a G protein-coupled receptor having at least about 75%, at least about 80%, at least about 85%, at least about 90% or at least about 95% identity to SEQ ID NO: 2.

In some embodiments, the G protein-coupled receptor comprises the amino acid sequence of SEQ ID NO: 2.

In some embodiments, the G protein-coupled receptor comprises the amino acid sequence of a G protein-coupled receptor that is a constitutively active version of a receptor having SEQ ID NO: 2.

In some embodiments, PCR is RT-PCR.

In some embodiments, the human DNA is human cDNA derived from a tissue or cell type that expresses BRS-3. In some embodiments, the human cDNA is derived from hypothalamus.

In some embodiments, the G protein-coupled receptor encoded by a polynucleotide that is amplifiable by polymerase chain reaction (PCR) on a human DNA sample using specific primers SEQ ID NO: 3 and SEQ ID NO: 4 is an endogenous BRS-3 G protein-coupled receptor.

In some embodiments, the GPCR is recombinant.

In some embodiments, the host cell is a recombinant host cell.

In some embodiments, the GPCR is endogenous. In some embodiments, the GPCR that is endogenous is an endogenous mammalian GPCR. In some embodiments, the GPCR that is an endogenous mammalian GPCR is an endogenous mammalian BRS-3. In some embodiments, the GPCR is non-endogenous.

In some embodiments, the GPCR is a mammalian BRS-3.

In some embodiments, said host cell comprises an expression vector comprising a polynucleotide encoding the GPCR.

In some embodiments, said determining is carried out with membrane comprising the GPCR.

In some embodiments, the candidate compound is a small molecule. In some embodiments, the candidate compound is a polypeptide. In some embodiments, the candidate compound is not an antibody or an antigen-binding fragment thereof. In some embodiments, the candidate compound is a polypeptide, provided that the polypeptide is not an antibody or an antigen-binding fragment thereof. In some embodiments, the candidate compound is an antibody or an antigen-binding fragment thereof. In some embodiments, the candidate compound is a lipid. In some embodiments, the candidate compound is not a polypeptide. In some embodiments, the candidate compound is not a peptoid. In some embodiments, the candidate compound is not a lipid. In some embodiments, the candidate compound is non-endogenous. In some embodiments, the candidate compound is not material that a prokaryote or eukaryote naturally produces. In some embodiments, the candidate compound is not material that a prokaryote naturally produces. In some embodiments, the candidate compound is not material that a eukaryote naturally produces. In some embodiments, the candidate compound is not material that a mammal naturally produces. In some embodiments, the candidate compound is a compound not known to inhibit or stimulate functionality of the GPCR. In some embodiments, the candidate compound is a compound not known to be an agonist of the GPCR. In some embodiments, the candidate compound is a compound not known to be a partial agonist of the GPCR. In some embodiments, the candidate compound is a compound not known to be an inverse agonist of the GPCR. In some embodiments, the candidate compound is a compound not known to be an antagonist of the GPCR.

In some embodiments, said method further comprises synthesis of the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder. In some embodiments, the compound which inhibits functionality of the GPCR in step (b) or the compound suitable for promoting sleep or for preventing or treating a sleep disorder ameliorated by promoting sleep, an anxiety disorder, a convulsive disorder, migraine, a depressive disorder, or a psychotic disorder is an inverse agonist or an antagonist of the GPCR.

In some embodiments, said method further comprises: optionally, determining the structure of the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder; and providing the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder or the name or structure of the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder. In some embodiments, the compound which inhibits functionality of the GPCR in step (b) or the compound suitable for promoting sleep or for preventing or treating a sleep disorder ameliorated by promoting sleep, an anxiety disorder, a convulsive disorder, migraine, a depressive disorder, or a psychotic disorder is an inverse agonist or an antagonist of the GPCR.

In some embodiments, said method further comprises: optionally, determining the structure of the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder; optionally, providing the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder or the name or structure of the compound which inhibits functionality of the GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder; and producing or synthesizing the compound which inhibits functionality of GPCR in step (b) or the compound in the presence of which less of said complex is formed in step (c′) or the compound suitable for promoting sleep or for preventing or treating a sleep disorder ameliorated by promoting sleep, an anxiety disorder, a convulsive disorder, migraine, a depressive disorder, or a psychotic disorder. In some embodiments, the compound which inhibits functionality of the GPCR in step (b) or the compound suitable for promoting sleep or for preventing or treating a sleep disorder ameliorated by promoting sleep, an anxiety disorder, a convulsive disorder, migraine, a depressive disorder, or a psychotic disorder is an inverse agonist or an antagonist of the GPCR.

In a second aspect, the invention features a compound suitable for promoting sleep or for preventing or treating a sleep disorder ameliorated by promoting sleep, an anxiety disorder, a convulsive disorder, migraine, a depressive disorder, or a psychotic disorder identifiable according to a method of the first aspect.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is identified according to a method of the first aspect.

In some embodiments, the sleep disorder comprises fragmented sleep architecture.

In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of the GPCR. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist of the GPCR. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an antagonist of the GPCR. In some embodiments, the inverse agonist or antagonist of the GPCR is an inverse agonist or antagonist of a mammalian BRS-3. In some embodiments, the inverse agonist or antagonist of the GPCR that is an inverse agonist or antagonist of a mammalian BRS-3 is a BRS-3 selective inverse agonist or antagonist. In some embodiments, the mammalian BRS-3 is a human BRS-3.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is a small molecule.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is a polypeptide. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is a polypeptide, provided that the polypeptide is not an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is a lipid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a polypeptide. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a peptoid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a lipid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is non-endogenous. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a prokaryote or eukaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a prokaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a eukaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a mammal naturally produces.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM at human BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 10 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 1 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 100 nM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 of less than about 1 μM, of less than about 100 nM, or of less than about 10 nM in said assay. In some embodiments, said modulator is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 10 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 1 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 100 nM.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is orally active.

In a third aspect, the invention features a pharmaceutical composition comprising a compound suitable for promoting sleep or for preventing or treating a sleep disorder ameliorated by promoting sleep, an anxiety disorder, a convulsive disorder, migraine, a depressive disorder, or a psychotic disorder and a pharmaceutically acceptable carrier.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of a mammalian BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of a human BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of human BRS-3 having the amino acid sequence of SEQ ID NO: 2. In some embodiments, the compound suitable for promoting sleep or for preventing or treating a sleep disorder ameliorated by promoting sleep, an anxiety disorder, a convulsive disorder, migraine, a depressive disorder, or a psychotic disorder that is an inverse agonist or antagonist of a mammalian or human BRS-3 is a BRS-3 selective inverse agonist or antagonist.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is according to the second aspect.

In some embodiments, the sleep disorder comprises fragmented sleep architecture.

In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is a small molecule.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is a polypeptide. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is a polypeptide, provided that the polypeptide is not an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is a lipid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a polypeptide. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a peptoid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a lipid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is non-endogenous. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a prokaryote or eukaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a prokaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a eukaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a mammal naturally produces.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM at human BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 10 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 1 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 100 nM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM in said assay. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 10 M. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 1 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 100 nM.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is orally active.

In a fourth aspect, the invention features a method of preparing a pharmaceutical composition comprising admixing a compound suitable for promoting sleep or for preventing or treating a sleep disorder ameliorated by promoting sleep, an anxiety disorder, a convulsive disorder, migraine, a depressive disorder, or a psychotic disorder and a pharmaceutically acceptable carrier.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of a mammalian BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of a human BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of human BRS-3 having the amino acid sequence of SEQ ID NO: 2. In some embodiments, the compound suitable for promoting sleep or for preventing or treating a sleep disorder ameliorated by promoting sleep, an anxiety disorder, a convulsive disorder, migraine, a depressive disorder, or a psychotic disorder that is an inverse agonist or antagonist of a mammalian or human BRS-3 is a BRS-3 selective inverse agonist or antagonist.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is according to the second aspect.

In some embodiments, the sleep disorder comprises fragmented sleep architecture.

In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist of the GPCR. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist of the GPCR. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an antagonist of the GPCR. In some embodiments, the inverse agonist or antagonist of the GPCR is an inverse agonist or antagonist of a mammalian BRS-3. In some embodiments, the inverse agonist or antagonist of the human BRS-3 is an inverse agonist or antagonist of human BRS-3 having the amino acid sequence of SEQ ID NO: 2.

In some embodiments, the inverse agonist or antagonist of the GPCR that is an inverse agonist or antagonist of a mammalian BRS-3 is a BRS-3 selective inverse agonist or antagonist. In some embodiments, the mammalian BRS-3 is a human BRS-3.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is a small molecule.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is a polypeptide. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is a polypeptide, provided that the polypeptide is not an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an antibody or an antigen-binding fragment thereof. In some embodiments, the compound suitable for promoting sleep or for preventing or treating a sleep disorder ameliorated by promoting sleep, an anxiety disorder, a convulsive disorder, migraine, a depressive disorder, or a psychotic disorder is a lipid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a polypeptide. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a peptoid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not a lipid. In some embodiments, the compound suitable for promoting sleep or for preventing or treating a sleep disorder ameliorated by promoting sleep, an anxiety disorder, a convulsive disorder, migraine, a depressive disorder, or a psychotic disorder is non-endogenous. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a prokaryote or eukaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a prokaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a eukaryote naturally produces. In some embodiments, the compound suitable for promoting sleep or for preventing or treating a sleep disorder ameliorated by promoting sleep, an anxiety disorder, a convulsive disorder, migraine, a depressive disorder, or a psychotic disorder is not material that a mammal naturally produces.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM at human BRS-3. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 10 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 1 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than a value selected from the interval of about 10 nM to 100 nM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than about 10 μM, of less than about 1 μM, of less than about 100 nM, or of less than about 10 nM in said assay. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 10 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 1 μM. In some embodiments, the compound suitable for promoting sleep or for preventing or treating a sleep disorder ameliorated by promoting sleep, an anxiety disorder, a convulsive disorder, migraine, a depressive disorder, or a psychotic disorder is an inverse agonist or antagonist with an IC₅₀ in said assay of less than a value selected from the interval of about 10 nM to 100 nM.

In some embodiments, the compound suitable for promoting sleep or for preventing or treating a sleep disorder ameliorated by promoting sleep, an anxiety disorder, a convulsive disorder, migraine, a depressive disorder, or a psychotic disorder is orally active.

In a fifth aspect, the invention features a method of promoting sleep or of preventing or treating a sleep disorder ameliorated by promoting sleep, an anxiety disorder, a convulsive disorder, migraine, a depressive disorder, or a psychotic disorder comprising administering to a mammal in need thereof a therapeutically effective amount of an inverse agonist or antagonist of the mammalian BRS-3 or a pharmaceutically acceptable composition comprising the inverse agonist or antagonist and a pharmaceutically acceptable carrier.

In some embodiments, the mammal is a human.

In some embodiments, the inverse agonist or antagonist of the mammalian BRS-3 is an inverse agonist or antagonist of a human BRS-3. In some embodiments, the inverse agonist or antagonist of the human BRS-3 is an inverse agonist or antagonist of human BRS-3 having the amino acid sequence of SEQ ID NO: 2. In some embodiments, the inverse agonist or antagonist of a mammalian or human BRS-3 is a BRS-3 selective inverse agonist or antagonist.

In some embodiments, the inverse agonist or antagonist of the mammalian BRS-3 is according to the second aspect.

In some embodiments, the sleep disorder comprises fragmented sleep architecture.

In some embodiments, said promoting sleep or preventing or treating a sleep disorder ameliorated by promoting sleep comprises promoting sleep consolidation.

In some embodiments, said promoting sleep or preventing or treating a sleep disorder ameliorated by promoting sleep comprises increasing delta power.

In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the inverse agonist or antagonist of the mammalian BRS-3 is a small molecule.

In some embodiments, the inverse agonist or antagonist of the mammalian BRS-3 is a polypeptide. In some embodiments, the inverse agonist or antagonist of the mammalian BRS-3 is not an antibody or an antigen-binding fragment thereof. In some embodiments, the inverse agonist or antagonist of the mammalian BRS-3 is a polypeptide, provided that the polypeptide is not an antibody or an antigen-binding fragment thereof. In some embodiments, the inverse agonist or antagonist of the mammalian BRS-3 is an antibody or an antigen-binding fragment thereof. In some embodiments, the inverse agonist or antagonist of the mammalian BRS-3 is a lipid. In some embodiments, the inverse agonist or antagonist of the mammalian BRS-3 is not a polypeptide. In some embodiments, the inverse agonist or antagonist of the mammalian BRS-3 is not a peptoid. In some embodiments, the inverse agonist or antagonist of the mammalian BRS-3 is not a lipid. In some embodiments, the inverse agonist or antagonist of the mammalian BRS-3 is non-endogenous. In some embodiments, the inverse agonist or antagonist of the mammalian BRS-3 is not material that a prokaryote or eukaryote naturally produces. In some embodiments, the inverse agonist or antagonist of the mammalian BRS-3 is not material that a prokaryote naturally produces. In some embodiments, the inverse agonist or antagonist of the mammalian BRS-3 is not material that a eukaryote naturally produces. In some embodiments, the inverse agonist or antagonist of the mammalian BRS-3 is not material that a mammal naturally produces.

In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM at human BRS-3. In some embodiments, the inverse agonist or antagonist of the mammalian BRS-3 is an inverse agonist or antagonist with an IC₅₀ of less than a value selected from the interval of about 10 nM to 10 μM. In some embodiments, the inverse agonist or antagonist of the mammalian BRS-3 is an inverse agonist or antagonist with an IC₅₀ of less than a value selected from the interval of about 10 nM to 1 μM. In some embodiments, the inverse agonist or antagonist of the mammalian BRS-3 is an inverse agonist or antagonist with an IC₅₀ of less than a value selected from the interval of about 10 nM to 100 nM. In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM in said assay. In some embodiments, the inverse agonist or antagonist of the mammalian BRS-3 is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the inverse agonist or antagonist of the mammalian BRS-3 is an inverse agonist or antagonist with an IC₅₀ in said assay of less than a value selected from the interval of about 10 nM to 10 μM. In some embodiments, the inverse agonist or antagonist of the mammalian BRS-3 is an inverse agonist or antagonist with an IC₅₀ in said assay of less than a value selected from the interval of about 10 nM to 1 In some embodiments, the inverse agonist or antagonist of the mammalian BRS-3 is an inverse agonist or antagonist with an IC₅₀ in said assay of less than a value selected from the interval of about 10 nM to 100 nM.

In some embodiments, the inverse agonist or antagonist of the mammalian BRS-3 is a compound selected from Table E. In some embodiments, the inverse agonist or antagonist of the mammalian BRS-3 is Compound E21. In some embodiments, the inverse agonist or antagonist of the mammalian BRS-3 is Compound E22.

In some embodiments, the antagonist of the mammalian BRS-3 is a compound selected from Table E. In some embodiments, the antagonist of the mammalian BRS-3 is Compound E21. In some embodiments, the antagonist of the mammalian BRS-3 is Compound E22.

In some embodiments, the inverse agonist or antagonist of the mammalian BRS-3 is orally active.

In a sixth aspect, the invention features use of an inverse agonist or antagonist of a mammalian BRS-3 for the manufacture of a medicament for promoting sleep or for preventing or treating a sleep disorder ameliorated by promoting sleep, an anxiety disorder, a convulsive disorder, migraine, a depressive disorder, or a psychotic disorder.

In some embodiments, the use of an inverse agonist or antagonist of a mammalian BRS-3 for the manufacture of a medicament for promoting sleep or for preventing or treating a sleep disorder ameliorated by promoting sleep, an anxiety disorder, a convulsive disorder, migraine, a depressive disorder, or a psychotic disorder is a use of an inverse agonist or antagonist of a mammalian BRS-3 for the manufacture of a medicament for promoting sleep or for preventing or treating a sleep disorder ameliorated by promoting sleep, an anxiety disorder, a convulsive disorder, migraine, a depressive disorder, or a psychotic disorder in the mammal.

In some embodiments, the mammalian BRS-3 is a human BRS-3. In some embodiments, the mammal is a human.

In some embodiments, the inverse agonist or antagonist of the mammalian BRS-3 is an inverse agonist or antagonist of a human BRS-3. In some embodiments, the inverse agonist or antagonist of the human BRS-3 is an inverse agonist or antagonist of human BRS-3 having the amino acid sequence of SEQ ID NO: 2. In some embodiments, the inverse agonist or antagonist of a mammalian or human BRS-3 is a BRS-3 selective inverse agonist or antagonist.

In some embodiments, the inverse agonist or antagonist of the mammalian BRS-3 is according to the second aspect.

In some embodiments, the sleep disorder comprises fragmented sleep architecture.

In some embodiments, said promoting sleep or preventing or treating a sleep disorder ameliorated by promoting sleep comprises promoting sleep consolidation.

In some embodiments, said promoting sleep or preventing or treating a sleep disorder ameliorated by promoting sleep comprises increasing delta power.

In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the inverse agonist or antagonist of the mammalian BRS-3 is a small molecule.

In some embodiments, the inverse agonist or antagonist of the mammalian BRS-3 is a polypeptide. In some embodiments, the inverse agonist or antagonist of the mammalian BRS-3 is not an antibody or an antigen-binding fragment thereof. In some embodiments, the inverse agonist or antagonist of the mammalian BRS-3 is a polypeptide, provided that the polypeptide is not an antibody or an antigen-binding fragment thereof. In some embodiments, the inverse agonist or antagonist of the mammalian BRS-3 is an antibody or an antigen-binding fragment thereof. In some embodiments, the inverse agonist or antagonist of the mammalian BRS-3 is a lipid. In some embodiments, the inverse agonist or antagonist of the mammalian BRS-3 is not a polypeptide. In some embodiments, the inverse agonist or antagonist of the mammalian BRS-3 is not a peptoid. In some embodiments, the inverse agonist or antagonist of the mammalian BRS-3 is not a lipid. In some embodiments, the inverse agonist or antagonist of the mammalian BRS-3 is non-endogenous. In some embodiments, the inverse agonist or antagonist of the mammalian BRS-3 is not material that a prokaryote or eukaryote naturally produces. In some embodiments, the inverse agonist or antagonist of the mammalian BRS-3 is not material that a prokaryote naturally produces. In some embodiments, the inverse agonist or antagonist of the mammalian BRS-3 is not material that a eukaryote naturally produces. In some embodiments, the inverse agonist or antagonist of the mammalian BRS-3 is not material that a mammal naturally produces.

In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM at human BRS-3. In some embodiments, the inverse agonist or antagonist of the mammalian BRS-3 is an inverse agonist or antagonist with an IC₅₀ of less than a value selected from the interval of about 10 nM to 10 μM. In some embodiments, the inverse agonist or antagonist of the mammalian BRS-3 is an inverse agonist or antagonist with an IC₅₀ of less than a value selected from the interval of about 10 nM to 1 μM. In some embodiments, the inverse agonist or antagonist of the mammalian BRS-3 is an inverse agonist or antagonist with an IC₅₀ of less than a value selected from the interval of about 10 nM to 100 nM. In some embodiments, the inverse agonist or antagonist of the mammalian. BRS-3 is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM in said assay. In some embodiments, the inverse agonist or antagonist of the mammalian BRS-3 is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the inverse agonist or antagonist of the mammalian BRS-3 is an inverse agonist or antagonist with an IC₅₀ in said assay of less than a value selected from the interval of about 10 nM to 10 μM. In some embodiments, the inverse agonist or antagonist of the mammalian BRS-3 is an inverse agonist or antagonist with an IC₅₀ in said assay of less than a value selected from the interval of about 10 nM to 1 μM. In some embodiments, the inverse agonist or antagonist of the mammalian BRS-3 is an inverse agonist or antagonist with an IC₅₀ in said assay of less than a value selected from the interval of about 10 nM to 100 nM.

In some embodiments, the inverse agonist or antagonist of the mammalian BRS-3 is a compound selected from Table E. In some embodiments, the inverse agonist or antagonist of the mammalian BRS-3 is Compound E21. In some embodiments, the inverse agonist or antagonist of the mammalian BRS-3 is Compound E22.

In some embodiments, the antagonist of the mammalian BRS-3 is a compound selected from Table E. In some embodiments, the antagonist of the mammalian BRS-3 is Compound E21. In some embodiments, the antagonist of the mammalian BRS-3 is Compound E22.

In some embodiments, the inverse agonist or antagonist of the mammalian BRS-3 is orally active.

In a seventh aspect, the invention features an inverse agonist or antagonist of a mammalian BRS-3 or a pharmaceutical composition comprising the inverse agonist or antagonist and a pharmaceutically acceptable carrier for use to promote sleep or to prevent or treat a sleep disorder ameliorated by promoting sleep, an anxiety disorder, a convulsive disorder, migraine, a depressive disorder, or a psychotic disorder.

In some embodiments, the mammalian BRS-3 is a human BRS-3. In some embodiments, the mammal is a human.

In some embodiments, the inverse agonist or antagonist of a mammalian BRS-3 or a pharmaceutical composition comprising the inverse agonist or antagonist and a pharmaceutically acceptable carrier for use to promote sleep or to prevent or treat a sleep disorder ameliorated by promoting sleep, an anxiety disorder, a convulsive disorder, migraine, a depressive disorder, or a psychotic disorder is an inverse agonist or antagonist of a mammalian BRS-3 for use to promote sleep or to prevent or treat a sleep disorder ameliorated by promoting sleep, an anxiety disorder, a convulsive disorder, migraine, a depressive disorder, or a psychotic disorder. In some embodiments, the inverse agonist or antagonist of a mammalian BRS-3 or a pharmaceutical composition comprising the inverse agonist or antagonist and a pharmaceutically acceptable carrier for use to promote sleep or to prevent or treat a sleep disorder ameliorated by promoting sleep, an anxiety disorder, a convulsive disorder, migraine, a depressive disorder, or a psychotic disorder is the pharmaceutical composition comprising the inverse agonist or antagonist of a mammalian BRS-3 and a pharmaceutically acceptable carrier for use to promote sleep or to prevent or treat a sleep disorder ameliorated by promoting sleep, an anxiety disorder, a convulsive disorder, migraine, a depressive disorder, or a psychotic disorder.

In some embodiments, the inverse agonist or antagonist of a mammalian BRS-3 or a pharmaceutical composition comprising the inverse agonist or antagonist and a pharmaceutically acceptable carrier for use to promote sleep or to prevent or treat a sleep disorder ameliorated by promoting sleep, an anxiety disorder, a convulsive disorder, migraine, a depressive disorder, or a psychotic disorder is an inverse agonist or antagonist of a mammalian BRS-3 or a pharmaceutical composition comprising the inverse agonist or antagonist and a pharmaceutically acceptable carrier for use to promote sleep or to prevent or treat a sleep disorder ameliorated by promoting sleep, an anxiety disorder, a convulsive disorder, migraine, a depressive disorder, or a psychotic disorder in the mammal.

In some embodiments, the inverse agonist or antagonist of the mammalian BRS-3 is an inverse agonist or antagonist of a human BRS-3. In some embodiments, the inverse agonist or antagonist of the human BRS-3 is an inverse agonist or antagonist of human BRS-3 having the amino acid sequence of SEQ ID NO: 2. In some embodiments, the inverse agonist or antagonist of a mammalian or human BRS-3 is a BRS-3 selective inverse agonist or antagonist.

In some embodiments, the inverse agonist or antagonist of the mammalian BRS-3 is according to the second aspect.

In some embodiments, the sleep disorder comprises fragmented sleep architecture.

In some embodiments, said promoting sleep or preventing or treating a sleep disorder ameliorated by promoting sleep comprises promoting sleep consolidation.

In some embodiments, said promoting sleep or preventing or treating a sleep disorder ameliorated by promoting sleep comprises increasing delta power.

In some embodiments, the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

In some embodiments, the sleep disorder is insomnia.

In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia.

In some embodiments, the inverse agonist or antagonist of the mammalian BRS-3 is a small molecule.

In some embodiments, the inverse agonist or antagonist of the mammalian BRS-3 is a polypeptide. In some embodiments, the inverse agonist or antagonist of the mammalian BRS-3 is not an antibody or an antigen-binding fragment thereof. In some embodiments, the inverse agonist or antagonist of the mammalian BRS-3 is a polypeptide, provided that the polypeptide is not an antibody or an antigen-binding fragment thereof. In some embodiments, the inverse agonist or antagonist of the mammalian BRS-3 is an antibody or an antigen-binding fragment thereof. In some embodiments, the inverse agonist or antagonist of the mammalian BRS-3 is a lipid. In some embodiments, the inverse agonist or antagonist of the mammalian BRS-3 is not a polypeptide. In some embodiments, the inverse agonist or antagonist of the mammalian BRS-3 is not a peptoid. In some embodiments, the inverse agonist or antagonist of the mammalian BRS-3 is not a lipid. In some embodiments, the inverse agonist or antagonist of the mammalian BRS-3 is non-endogenous. In some embodiments, the inverse agonist or antagonist of the mammalian BRS-3 is not material that a prokaryote or eukaryote naturally produces. In some embodiments, the inverse agonist or antagonist of the mammalian BRS-3 is not material that a prokaryote naturally produces. In some embodiments, the inverse agonist or antagonist of the mammalian BRS-3 is not material that a eukaryote naturally produces. In some embodiments, the inverse agonist or antagonist of the mammalian BRS-3 is not material that a mammal naturally produces.

In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM at human BRS-3. In some embodiments, the inverse agonist or antagonist of the mammalian BRS-3 is an inverse agonist or antagonist with an IC₅₀ of less than a value selected from the interval of about 10 nM to 10 μM. In some embodiments, the inverse agonist or antagonist of the mammalian BRS-3 is an inverse agonist or antagonist with an IC₅₀ of less than a value selected from the interval of about 10 nM to 1 μM. In some embodiments, the inverse agonist or antagonist of the mammalian BRS-3 is an inverse agonist or antagonist with an IC₅₀ of less than a value selected from the interval of about 10 nM to 100 nM. In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the inverse agonist or antagonist of the mammalian BRS-3 is an inverse agonist or antagonist with an IC₅₀ of less than about 10 μM, of less than about 1 μM, of less than about 100 nM, or of less than about 10 nM in said assay. In some embodiments, the inverse agonist or antagonist of the mammalian BRS-3 is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 AM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the inverse agonist or antagonist of the mammalian BRS-3 is an inverse agonist or antagonist with an IC₅₀ in said assay of less than a value selected from the interval of about 10 nM to 10 μM. In some embodiments, the inverse agonist or antagonist of the mammalian BRS-3 is an inverse agonist or antagonist with an IC₅₀ in said assay of less than a value selected from the interval of about 10 nM to 1 μM. In some embodiments, the inverse agonist or antagonist of the mammalian BRS-3 is an inverse agonist or antagonist with an IC₅₀ in said assay of less than a value selected from the interval of about 10 nM to 100 nM. In some embodiments, the inverse agonist or antagonist of the mammalian BRS-3 is a compound selected from Table E. In some embodiments, the inverse agonist or antagonist of the mammalian BRS-3 is Compound E21. In some embodiments, the inverse agonist or antagonist of the mammalian BRS-3 is Compound E22.

In some embodiments, the antagonist of the mammalian BRS-3 is a compound selected from Table E. In some embodiments, the antagonist of the mammalian BRS-3 is Compound E21. In some embodiments, the antagonist of the mammalian BRS-3 is Compound E22.

In some embodiments, the inverse agonist or antagonist of the mammalian BRS-3 is orally active.

In an eighth aspect, the invention features a method for identifying compounds suitable for promoting wakefulness or for preventing or treating excessive sleepiness or for preventing or treating a GABA-related neurological disorder selected from the group consisting of a sleep disorder ameliorated by promoting wakefulness and a cognitive disorder, comprising the steps of:

(a) contacting a candidate compound with a host cell or with membrane of a host cell that expresses a GPCR, wherein the GPCR comprises an amino acid sequence selected from the group consisting of:
(i) the amino acid sequence of SEQ ID NO: 2;
(ii) amino acids 2-399 of SEQ ID NO: 2;
(iii) the amino acid sequence of a G protein-coupled receptor encoded by a polynucleotide that is amplifiable by polymerase chain reaction (PCR) on a human DNA sample using specific primers SEQ ID NO: 3 and SEQ ID NO: 4;
(iv) the amino acid sequence of a G protein-coupled receptor encoded by a polynucleotide that hybridizes under stringent conditions to the complement of SEQ ID NO: 1;
(v) the amino acid sequence of a G protein-coupled receptor having an amino acid sequence derived from SEQ ID NO: 2 by substitution, deletion or addition of one or several amino acids in the amino acid sequence of SEQ ID NO: 2;
(vi) the amino acid sequence of a G protein-coupled receptor having at least about 75%, at least about 80%, at least about 85%, at least about 90% or at least about 95% identity to SEQ ID NO: 2;
(vii) the amino acid sequence of a G protein-coupled receptor that is a constitutively active version of a receptor having SEQ ID NO: 2; and
(viii) a biologically active fragment of any one of (i) to (vii); and

(b) determining the ability of the candidate compound to stimulate functionality of the receptor;

wherein the ability of the candidate compound to stimulate functionality of the GPCR is indicative of the candidate compound being a compound suitable for promoting wakefulness or for preventing or treating excessive sleepiness or for preventing or treating a GABA-related neurological disorder selected from the group consisting of a sleep disorder ameliorated by promoting wakefulness and a cognitive disorder.

In certain embodiments, said method is a method for identifying compounds suitable for promoting wakefulness. In certain embodiments, said method is a method for identifying compounds suitable for preventing or treating excessive sleepiness. In certain embodiments, said method is a method for identifying compounds suitable for preventing or treating a GABA-related neurological disorder selected from the group consisting of a sleep disorder ameliorated by promoting wakefulness and a cognitive disorder. In certain embodiments, said method is a method for identifying compounds suitable for preventing or treating a sleep disorder ameliorated by promoting wakefulness. In certain embodiments, said method is a method for identifying compounds suitable for preventing or treating a cognitive disorder.

The invention additionally features a method for identifying compounds suitable for promoting wakefulness or for preventing or treating excessive sleepiness or for preventing or treating a GABA-related neurological disorder selected from the group consisting of a sleep disorder ameliorated by promoting wakefulness, or a cognitive disorder, comprising steps (a) and (b) of this eighth aspect, and further comprising:
(c) optionally synthesizing a compound which stimulates functionality of the GPCR in step (b);
(d) administering a compound which stimulates functionality of the GPCR in step (b) to a mammal; and
(e) determining whether the compound promotes wakefulness or has cognition-enhancing activity in the mammal;
   wherein the ability of the candidate compound to promote wakefulness or to show cognition-enhancing activity in the mammal is indicative of the candidate compound being a compound suitable for promoting wakefulness or for preventing or treating excessive sleepiness or for preventing or treating a GABA-related neurological disorder selected from the group consisting of a sleep disorder ameliorated by promoting wakefulness and a cognitive disorder.

In certain embodiments, said method is a method for identifying compounds suitable for promoting wakefulness. In certain embodiments, said method is a method for identifying compounds suitable for preventing or treating excessive sleepiness. In certain embodiments, said method is a method for identifying compounds suitable for preventing or treating a GABA-related neurological disorder selected from the group consisting of a sleep disorder ameliorated by promoting wakefulness and a cognitive disorder. In certain embodiments, said method is a method for identifying compounds suitable for preventing or treating a sleep disorder ameliorated by promoting wakefulness. In certain embodiments, said method is a method for identifying compounds suitable for preventing or treating a cognitive disorder.

In some embodiments, the mammal is a human. In some embodiments, the mammal is a non-human mammal. In some embodiments, the non-human mammal is a laboratory animal. In some embodiments, the non-human mammal is a non-human primate. In some embodiments, the non-human mammal is a rodent. In some embodiments, the non-human mammal is a rat. In some embodiments, the non-human mammal is a mouse.

In some embodiments, said determining whether the compound promotes wakefulness in the mammal comprises polysomnography.

In some embodiments, the method comprises identifying an agonist of the GPCR. In some embodiments, said method further comprises formulating the agonist as a pharmaceutical.

In some embodiments, the method comprises identifying a partial agonist of the GPCR. In some embodiments, said method further comprises formulating the partial agonist as a pharmaceutical.

In some embodiments, said contacting comprises contacting in the absence of a known ligand of the GPCR. In some embodiments, said contacting comprises contacting in the absence of a known ligand of endogenous human BRS-3. In some embodiments, said contacting comprises contacting in the absence of a known agonist of the GPCR. In some embodiments, said contacting comprises contacting in the absence of a known agonist of endogenous human BRS-3.

In some embodiments, the method comprises detecting a second messenger.

In some embodiments, said determining is by a process comprising the measurement of a level of a second messenger selected from the group consisting of cyclic AMP (cAMP), cyclic GMP (cGMP), inositol 1,4,5-triphosphate (IP3), diacylglycerol (DAG), MAP kinase activity, MAPK/ERK kinase kinase-1 (MEKK1) activity, and Ca²⁺. In some embodiments, said second messenger is IP3. In some embodiments, the level of intracellular IP3 is increased. In some embodiments, said second messenger is Ca²⁺. In some embodiments, the level of intracellular Ca²⁺ is increased.

In some embodiments, said determining is by a process comprising the use of a Melanophore assay. In some embodiments, the melanophore cells undergo pigment dispersion. In some embodiments, the candidate compound stimulates pigment dispersion.

In some embodiments, said determining is by a process comprising the measurement of GTPγS binding to membrane comprising the GPCR. In some embodiments, GTPγS binding to membrane comprising the GPCR is increased.

In some embodiments, the method further comprises the step of comparing the modulation of the GPCR caused by the candidate compound to a second modulation of the GPCR caused by contacting the GPCR with a known modulator of the GPCR.

In some embodiments, the baseline intracellular response is stimulated in the presence of the candidate compound by at least about 10%, by a least about 25%, by at least about 50%, by a least about 100%, by at least about 200%, by at least about 300%, by at least about 400%, or by least about 500% as compared with the baseline response in the absence of the candidate compound.

In some embodiments, the baseline intracellular response is stimulated in the presence of the agonist or partial agonist by at least about 10%, by a least about 25%, by at least about 50%, by a least about 100%, by at least about 200%, by at least about 300%, by at least about 400%, or by least about 500% as compared with the baseline response in the absence of the agonist or partial agonist.

The invention also relates to a method for identifying compounds suitable for promoting wakefulness or for preventing or treating excessive sleepiness or for preventing or treating a GABA-related neurological disorder selected from the group consisting of a sleep disorder ameliorated by promoting wakefulness and a cognitive disorder, comprising the steps of:

(a′) contacting a host cell or membrane of a host cell that expresses a GPCR with an optionally labeled known ligand to the GPCR in the presence or absence of a candidate compound, wherein the GPCR comprises an amino acid sequence selected from the group consisting of:
(i) the amino acid sequence of SEQ ID NO: 2;
(ii) amino acids 2-399 of SEQ ID NO: 2;
(iii) the amino acid sequence of a G protein-coupled receptor encoded by a polynucleotide that is amplifiable by polymerase chain reaction (PCR) on a human DNA sample using specific primers SEQ ID NO: 3 and SEQ ID NO: 4;
(iv) the amino acid sequence of a G protein-coupled receptor encoded by a polynucleotide that hybridizes under stringent conditions to the complement of SEQ ID NO: 1;
(v) the amino acid sequence of a G protein-coupled receptor having an amino acid sequence derived from SEQ ID NO: 2 by substitution, deletion or addition of one or several amino acids in the amino acid sequence of SEQ ID NO: 2;
(vi) the amino acid sequence of a G protein-coupled receptor having at least about 75%, at least about 80%, at least about 85%, at least about 90% or at least about 95% identity to SEQ ID NO: 2;
(vii) the amino acid sequence of a G protein-coupled receptor that is a constitutively active version of a receptor having SEQ ID NO: 2; and
(viii) a biologically active fragment of any one of (i) to (vii); and

(b′) detecting the complex between said known ligand and said GPCR;

(c′) determining whether less of said complex is formed in the presence of the candidate compound than in the absence of the candidate compound;

(d′) optionally synthesizing a compound in the presence of which less of said complex is formed in step (c′);

(e′) administering a compound in the presence of which less of said complex is formed in step (c′) to a mammal; and

(f) determining whether the compound promotes wakefulness or has cognition-enhancing activity in the mammal;

wherein the ability of the candidate compound to promote wakefulness or to show cognition-enhancing activity in the mammal is indicative of the candidate compound being a compound suitable for promoting wakefulness or for preventing or treating excessive sleepiness or for preventing or treating a GABA-related neurological disorder selected from the group consisting of a sleep disorder ameliorated by promoting wakefulness and a cognitive disorder.

In certain embodiments, said method is a method for identifying compounds suitable for promoting wakefulness. In certain embodiments, said method is a method for identifying compounds suitable for preventing or treating excessive sleepiness. In certain embodiments, said method is a method for identifying compounds suitable for preventing or treating a GABA-related neurological disorder selected from the group consisting of a sleep disorder ameliorated by promoting wakefulness and a cognitive disorder. In certain embodiments, said method is a method for identifying compounds suitable for preventing or treating a sleep disorder ameliorated by promoting wakefulness. In certain embodiments, said method is a method for identifying compounds suitable for preventing or treating a cognitive disorder.

In some embodiments, the mammal is a human. In some embodiments, the mammal is a non-human mammal. In some embodiments, the non-human mammal is a laboratory animal. In some embodiments, the non-human mammal is a non-human primate. In some embodiments, the non-human mammal is a rodent. In some embodiments, the non-human mammal is a rat. In some embodiments, the non-human mammal is a mouse.

In some embodiments, said method is for isolating compounds suitable for promoting wakefulness or for preventing or treating excessive sleepiness or for preventing or treating a GABA-related neurological disorder selected from a sleep disorder ameliorated by promoting wakefulness and a cognitive disorder.

In some embodiments, said determining whether the compound promotes wakefulness in the mammal comprises polysomnography.

In some embodiments, said optionally labeled known ligand is radiolabeled.

In some embodiments, the known ligand is a compound selected from Table D. In some embodiments, the known ligand is Compound D28, Compound D30, Compound D31, or Compound D34. In some embodiments, the known ligand is a compound selected from Table E. In some embodiments, the known ligand is Compound E21 or Compound E22.

In some embodiments, said determining whether less of said complex is formed in the presence in the presence of the candidate compound than in the absence of the candidate compound comprises determining whether at least about 10% less, at least about 20% less, at least about 30% less, at least about 40% less, at least about 50% less, at least about 60% less, at least about 70% less, at least about 75% less, at least about 80% less, at least about 85% less, at least about 90% less, or at least about 95% less of said complex is formed in the presence in the presence of the candidate compound than in the absence of the candidate compound.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder,

wherein the psychological disorder is selected from depression and schizophrenia. In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, the Cognitive Disorder is Dementia or Dementia of the Alzheimer's Type. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the host cell is a eukaryotic cell.

In some embodiments, the host cell is a mammalian cell. In some embodiments, the mammalian host cell is a CHO cell, a COS-7 cell, an MCB3901 cell, a 293 cell or a 293T cell.

In some embodiments, the host cell is a yeast cell.

In some embodiments, the host cell is a melanophore cell.

In some embodiments, the G protein-coupled receptor comprises the amino acid sequence of a G protein-coupled receptor having at least about 75%, at least about 80%, at least about 85%, at least about 90% or at least about 95% identity to SEQ ID NO: 2.

In some embodiments, the G protein-coupled receptor comprises the amino acid sequence of SEQ ID NO: 2.

In some embodiments, the G protein-coupled receptor comprises the amino acid sequence of a G protein-coupled receptor that is a constitutively active version of a receptor having SEQ ID NO: 2.

In some embodiments, PCR is RT-PCR.

In some embodiments, the human DNA is human cDNA derived from a tissue or cell type that expresses BRS-3. In some embodiments, the human cDNA is derived from hypothalamus.

In some embodiments, the G protein-coupled receptor encoded by a polynucleotide that is amplifiable by polymerase chain reaction (PCR) on a human DNA sample using specific primers SEQ ID NO: 3 and SEQ ID NO: 4 is an endogenous BRS-3 G protein-coupled receptor.

In some embodiments, the GPCR is recombinant.

In some embodiments, the host cell is a recombinant host cell.

In some embodiments, the GPCR is endogenous. In some embodiments, the GPCR that is endogenous is an endogenous mammalian GPCR. In some embodiments, the GPCR that is an endogenous mammalian GPCR is an endogenous mammalian BRS-3. In some embodiments, the GPCR is non-endogenous.

In some embodiments, the GPCR is a mammalian BRS-3.

In some embodiments, said host cell comprises an expression vector comprising a polynucleotide encoding the GPCR.

In some embodiments, said determining is carried out with membrane comprising the GPCR.

In some embodiments, the candidate compound is a small molecule.

In some embodiments, the candidate compound is a polypeptide. In some embodiments, the candidate compound is not an antibody or an antigen-binding fragment thereof. In some embodiments, the candidate compound is a polypeptide, provided that the polypeptide is not an antibody or an antigen-binding fragment thereof. In some embodiments, the candidate compound is an antibody or an antigen-binding fragment thereof. In some embodiments, the candidate compound is a lipid. In some embodiments, the candidate compound is not a polypeptide. In some embodiments, the candidate compound is not a peptoid. In some embodiments, the candidate compound is not a lipid. In some embodiments, the candidate compound is non-endogenous. In some embodiments, the candidate compound is not material that a prokaryote or eukaryote naturally produces. In some embodiments, the candidate compound is not material that a prokaryote naturally produces. In some embodiments, the candidate compound is not material that a eukaryote naturally produces. In some embodiments, the candidate compound is not material that a mammal naturally produces. In some embodiments, the candidate compound is a compound not known to inhibit or stimulate functionality of the GPCR. In some embodiments, the candidate compound is a compound not known to be an agonist of the GPCR. In some embodiments, the candidate compound is a compound not known to be a partial agonist of the GPCR. In some embodiments, the candidate compound is a compound not known to be an inverse agonist of the GPCR. In some embodiments, the candidate compound is a compound not known to be an antagonist of the GPCR.

In some embodiments, said method further comprises synthesis of the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder. In some embodiments, the compound which stimulates functionality of the GPCR in step (b) or the compound suitable for promoting wakefulness or for preventing or treating excessive sleepiness, a sleep disorder ameliorated by promoting wakefulness, or a cognitive disorder is an agonist or a partial agonist of the GPCR.

In some embodiments, said method further comprises: optionally, determining the structure of the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder; and providing the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder or the name or structure of the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder. In some embodiments, the compound which stimulates functionality of the GPCR in step (b) or the compound suitable for promoting wakefulness or for preventing or treating excessive sleepiness, a sleep disorder ameliorated by promoting wakefulness, or a cognitive disorder is an agonist or a partial agonist of the GPCR.

In some embodiments, said method further comprises: optionally, determining the structure of the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder; optionally, providing the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder or the name or structure of the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder; and producing or synthesizing the compound which stimulates functionality of the GPCR in step (b) or the compound in the presence of which less of said complex is formed in step (c′) or the compound suitable for promoting wakefulness or for preventing or treating excessive sleepiness, a sleep disorder ameliorated by promoting wakefulness, or a cognitive disorder. In some embodiments, the compound which stimulates functionality of the GPCR in step (b) or the compound suitable for promoting wakefulness or for preventing or treating excessive sleepiness, a sleep disorder ameliorated by promoting wakefulness, or a cognitive disorder is an agonist or a partial agonist of the GPCR.

In a ninth aspect, the invention features a compound suitable for promoting wakefulness or for preventing or treating excessive sleepiness, a sleep disorder ameliorated by promoting wakefulness, or a cognitive disorder identifiable according to a method of the eighth aspect.

In some embodiments, the compound suitable for promoting wakefulness or for preventing or treating excessive sleepiness, a sleep disorder ameliorated by promoting wakefulness, or a cognitive disorder is identified according to a method of the eighth aspect.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder, wherein the psychological disorder is selected from depression and schizophrenia.

In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the compound suitable for promoting wakefulness or for preventing or treating excessive sleepiness, a sleep disorder ameliorated by promoting wakefulness, or a cognitive disorder is an agonist or a partial agonist of the GPCR. In some embodiments, the compound suitable for promoting wakefulness or for preventing or treating excessive sleepiness, a sleep disorder ameliorated by promoting wakefulness, or a cognitive disorder is an agonist of the GPCR. In some embodiments, the compound suitable for promoting wakefulness or for preventing or treating excessive sleepiness, a sleep disorder ameliorated by promoting wakefulness, or a cognitive disorder is a partial agonist of the GPCR. In some embodiments, the agonist or partial agonist of the GPCR is an agonist or partial agonist of a mammalian BRS-3. In some embodiments, the agonist or partial agonist of the GPCR that is an agonist or partial agonist of a mammalian BRS-3 is a BRS-3 selective agonist or partial agonist. In some embodiments, the mammalian BRS-3 is a human BRS-3.

In some embodiments, the compound suitable for promoting wakefulness or for preventing or treating excessive sleepiness, a sleep disorder ameliorated by promoting wakefulness, or a cognitive disorder is a small molecule.

In some embodiments, the compound suitable for promoting wakefulness or for preventing or treating excessive sleepiness, a sleep disorder ameliorated by promoting wakefulness, or a cognitive disorder is a polypeptide. In some embodiments, the compound suitable for promoting wakefulness or for preventing or treating excessive sleepiness, a sleep disorder ameliorated by promoting wakefulness, or a cognitive disorder is not an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is a polypeptide, provided that the polypeptide is not an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is a lipid. In some embodiments, the compound suitable for promoting wakefulness or for preventing or treating excessive sleepiness, a sleep disorder ameliorated by promoting wakefulness, or a cognitive disorder is not a polypeptide. In some embodiments, the compound suitable for promoting wakefulness or for preventing or treating excessive sleepiness, a sleep disorder ameliorated by promoting wakefulness, or a cognitive disorder is not a peptoid. In some embodiments, the compound suitable for promoting wakefulness or for preventing or treating excessive sleepiness, a sleep disorder ameliorated by promoting wakefulness, or a cognitive disorder is not a lipid. In some embodiments, the compound suitable for promoting wakefulness or for preventing or treating excessive sleepiness, a sleep disorder ameliorated by promoting wakefulness, or a cognitive disorder is non-endogenous. In some embodiments, the compound suitable for promoting wakefulness or for preventing or treating excessive sleepiness, a sleep disorder ameliorated by promoting wakefulness, or a cognitive disorder is not material that a prokaryote or eukaryote naturally produces. In some embodiments, the compound suitable for promoting wakefulness or for preventing or treating excessive sleepiness, a sleep disorder ameliorated by promoting wakefulness, or a cognitive disorder is not material that a prokaryote naturally produces. In some embodiments, the compound suitable for promoting wakefulness or for preventing or treating excessive sleepiness, a sleep disorder ameliorated by promoting wakefulness, or a cognitive disorder is not material that a eukaryote naturally produces. In some embodiments, the compound suitable for promoting wakefulness or for preventing or treating excessive sleepiness, a sleep disorder ameliorated by promoting wakefulness, or a cognitive disorder is not material that a mammal naturally produces.

In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM at human BRS-3. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 10 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 1 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 100 nM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM in said assay. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 10 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 1 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 100 nM.

In some embodiments, the compound suitable for promoting wakefulness or for preventing or treating excessive sleepiness, a sleep disorder ameliorated by promoting wakefulness, or a cognitive disorder is orally active.

In a tenth aspect, the invention features a pharmaceutical composition comprising a compound suitable for promoting wakefulness or for preventing or treating excessive sleepiness, a sleep disorder ameliorated by promoting wakefulness, or a cognitive disorder and a pharmaceutically acceptable carrier.

In some embodiments, the compound suitable for promoting wakefulness or for preventing or treating excessive sleepiness, a sleep disorder ameliorated by promoting wakefulness, or a cognitive disorder is an agonist or partial agonist of a mammalian BRS-3. In some embodiments, the compound suitable for promoting wakefulness or for preventing or treating excessive sleepiness, a sleep disorder ameliorated by promoting wakefulness, or a cognitive disorder is an agonist or partial agonist of a human BRS-3. In some embodiments, the compound suitable for promoting wakefulness or for preventing or treating excessive sleepiness, a sleep disorder ameliorated by promoting wakefulness, or a cognitive disorder is an agonist or partial agonist of human BRS-3 having the amino acid sequence of SEQ ID NO: 2. In some embodiments, the compound suitable for promoting wakefulness or for preventing or treating excessive sleepiness, a sleep disorder ameliorated by promoting wakefulness, or a cognitive disorder that is an agonist or partial agonist of a mammalian or human BRS-3 is a BRS-3 selective agonist or partial agonist.

In some embodiments, the compound suitable for promoting wakefulness or for preventing or treating excessive sleepiness, a sleep disorder ameliorated by promoting wakefulness, or a cognitive disorder is according to the ninth aspect.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder, wherein the psychological disorder is selected from depression and schizophrenia.

In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the compound suitable for promoting wakefulness or for preventing or treating excessive sleepiness, a sleep disorder ameliorated by promoting wakefulness, or a cognitive disorder is a small molecule.

In some embodiments, the compound suitable for promoting wakefulness or for preventing or treating excessive sleepiness, a sleep disorder ameliorated by promoting wakefulness, or a cognitive disorder is a polypeptide. In some embodiments, the compound suitable for promoting wakefulness or for preventing or treating excessive sleepiness, a sleep disorder ameliorated by promoting wakefulness, or a cognitive disorder is not an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is a polypeptide, provided that the polypeptide is not an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is a lipid. In some embodiments, the compound suitable for promoting wakefulness or for preventing or treating excessive sleepiness, a sleep disorder ameliorated by promoting wakefulness, or a cognitive disorder is not a polypeptide. In some embodiments, the compound suitable for promoting wakefulness or for preventing or treating excessive sleepiness, a sleep disorder ameliorated by promoting wakefulness, or a cognitive disorder is not a peptoid. In some embodiments, the compound suitable for promoting wakefulness or for preventing or treating excessive sleepiness, a sleep disorder ameliorated by promoting wakefulness, or a cognitive disorder is not a lipid. In some embodiments, the compound suitable for promoting wakefulness or for preventing or treating excessive sleepiness, a sleep disorder ameliorated by promoting wakefulness, or a cognitive disorder is non-endogenous. In some embodiments, the compound suitable for promoting wakefulness or for preventing or treating excessive sleepiness, a sleep disorder ameliorated by promoting wakefulness, or a cognitive disorder is not material that a prokaryote or eukaryote naturally produces. In some embodiments, the compound suitable for promoting wakefulness or for preventing or treating excessive sleepiness, a sleep disorder ameliorated by promoting wakefulness, or a cognitive disorder is not material that a prokaryote naturally produces. In some embodiments, the compound suitable for promoting wakefulness or for preventing or treating excessive sleepiness, a sleep disorder ameliorated by promoting wakefulness, or a cognitive disorder is not material that a eukaryote naturally produces. In some embodiments, the compound suitable for promoting wakefulness or for preventing or treating excessive sleepiness, a sleep disorder ameliorated by promoting wakefulness, or a cognitive disorder is not material that a mammal naturally produces.

In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM at human BRS-3. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 10 ∥M. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 1 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 100 nM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM in said assay. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 10 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 1 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 100 nM.

In some embodiments, the compound suitable for promoting wakefulness or for preventing or treating excessive sleepiness, a sleep disorder ameliorated by promoting wakefulness, or a cognitive disorder is orally active.

In an eleventh aspect, the invention features a method of preparing a pharmaceutical composition comprising admixing a compound suitable for promoting wakefulness or for preventing or treating excessive sleepiness, a sleep disorder ameliorated by promoting wakefulness, or a cognitive disorder and a pharmaceutically acceptable carrier.

In some embodiments, the compound suitable for promoting wakefulness or for preventing or treating excessive sleepiness, a sleep disorder ameliorated by promoting wakefulness, or a cognitive disorder is an agonist or partial agonist of a mammalian BRS-3. In some embodiments, the compound suitable for promoting wakefulness or for preventing or treating excessive sleepiness, a sleep disorder ameliorated by promoting wakefulness, or a cognitive disorder is an agonist or partial agonist of a human BRS-3. In some embodiments, the compound suitable for promoting wakefulness or for preventing or treating excessive sleepiness, a sleep disorder ameliorated by promoting wakefulness, or a cognitive disorder is an agonist or partial agonist of human BRS-3 having the amino acid sequence of SEQ ID NO: 2. In some embodiments, the compound suitable for promoting wakefulness or for preventing or treating excessive sleepiness, a sleep disorder ameliorated by promoting wakefulness, or a cognitive disorder that is an agonist or partial agonist of a mammalian or human BRS-3 is a BRS-3 selective agonist or partial agonist.

In some embodiments, the compound suitable for promoting wakefulness or for preventing or treating excessive sleepiness, a sleep disorder ameliorated by promoting wakefulness, or a cognitive disorder is according to the ninth aspect.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder, wherein the psychological disorder is selected from depression and schizophrenia.

In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the compound suitable for promoting wakefulness or for preventing or treating excessive sleepiness, a sleep disorder ameliorated by promoting wakefulness, or a cognitive disorder is a small molecule.

In some embodiments, the compound suitable for promoting wakefulness or for preventing or treating excessive sleepiness, a sleep disorder ameliorated by promoting wakefulness, or a cognitive disorder is a polypeptide. In some embodiments, the compound suitable for promoting wakefulness or for preventing or treating excessive sleepiness, a sleep disorder ameliorated by promoting wakefulness, or a cognitive disorder is not an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is a polypeptide, provided that the polypeptide is not an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is an antibody or an antigen-binding fragment thereof. In some embodiments, the compound suitable for promoting wakefulness or for preventing or treating excessive sleepiness, a sleep disorder ameliorated by promoting wakefulness, or a cognitive disorder is a lipid. In some embodiments, the compound suitable for promoting wakefulness or for preventing or treating excessive sleepiness, a sleep disorder ameliorated by promoting wakefulness, or a cognitive disorder is not a polypeptide. In some embodiments, the compound suitable for promoting wakefulness or for preventing or treating excessive sleepiness, a sleep disorder ameliorated by promoting wakefulness, or a cognitive disorder is not a peptoid. In some embodiments, the compound suitable for promoting wakefulness or for preventing or treating excessive sleepiness, a sleep disorder ameliorated by promoting wakefulness, or a cognitive disorder is not a lipid. In some embodiments, the compound suitable for promoting wakefulness or for preventing or treating excessive sleepiness, a sleep disorder ameliorated by promoting wakefulness, or a cognitive disorder is non-endogenous. In some embodiments, the compound suitable for promoting wakefulness or for preventing or treating excessive sleepiness, a sleep disorder ameliorated by promoting wakefulness, or a cognitive disorder is not material that a prokaryote or eukaryote naturally produces. In some embodiments, the compound suitable for promoting wakefulness or for preventing or treating excessive sleepiness, a sleep disorder ameliorated by promoting wakefulness, or a cognitive disorder is not material that a prokaryote naturally produces. In some embodiments, the compound suitable for promoting wakefulness or for preventing or treating excessive sleepiness, a sleep disorder ameliorated by promoting wakefulness, or a cognitive disorder is not material that a eukaryote naturally produces. In some embodiments, the compound suitable for promoting wakefulness or for preventing or treating excessive sleepiness, a sleep disorder ameliorated by promoting wakefulness, or a cognitive disorder is not material that a mammal naturally produces.

In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM at human BRS-3. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 10 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 1 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than a value selected from the interval of about 10 nM to 100 nM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than about 10 μM, of less than about 1 μM, of less than about 100 nM, or of less than about 10 nM in said assay. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 10 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 1 μM. In some embodiments, the compound suitable for promoting wakefulness or for preventing or treating excessive sleepiness, a sleep disorder ameliorated by promoting wakefulness, or a cognitive disorder is an agonist or partial agonist with an EC₅₀ in said assay of less than a value selected from the interval of about 10 nM to 100 nM.

In some embodiments, the compound suitable for promoting wakefulness or for preventing or treating excessive sleepiness, a sleep disorder ameliorated by promoting wakefulness, or a cognitive disorder is orally active.

In a twelfth aspect, the invention features a method of promoting wakefulness or of preventing or treating excessive sleepiness, a sleep disorder ameliorated by promoting wakefulness, or a cognitive disorder comprising administering to a mammal in need thereof a therapeutically effective amount of an agonist or partial agonist of the mammalian BRS-3 or a pharmaceutically acceptable composition comprising the agonist or partial agonist and a pharmaceutically acceptable carrier.

In some embodiments, the mammal is a human.

In some embodiments, the agonist or partial agonist of the mammalian BRS-3 is an agonist or partial agonist of a human BRS-3. In some embodiments, the agonist or partial agonist of the human BRS-3 is an agonist or partial agonist of human BRS-3 having the amino acid sequence of SEQ ID NO: 2. In some embodiments, the agonist or partial agonist of a mammalian or human BRS-3 is a BRS-3 selective agonist or partial agonist.

In some embodiments, the agonist or partial agonist of the mammalian BRS-3 is according to the ninth aspect.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder, wherein the psychological disorder is selected from depression and schizophrenia.

In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the agonist or partial agonist of the mammalian BRS-3 is a small molecule.

In some embodiments, the agonist or partial agonist of the mammalian BRS-3 is a polypeptide. In some embodiments, the agonist or partial agonist of the mammalian BRS-3 is not an antibody or an antigen-binding fragment thereof. In some embodiments, the agonist or partial agonist of the mammalian BRS-3 is a polypeptide, provided that the polypeptide is not an antibody or an antigen-binding fragment thereof. In some embodiments, the agonist or partial agonist of the mammalian BRS-3 is an antibody or an antigen-binding fragment thereof. In some embodiments, the agonist or partial agonist of the mammalian BRS-3 is a lipid. In some embodiments, the agonist or partial agonist of the mammalian BRS-3 is not a polypeptide. In some embodiments, the agonist or partial agonist of the mammalian BRS-3 is not a peptoid. In some embodiments, the agonist or partial agonist of the mammalian BRS-3 is not a lipid. In some embodiments, the agonist or partial agonist of the mammalian BRS-3 is non-endogenous.

In some embodiments, the agonist or partial agonist of the mammalian BRS-3 is not material that a prokaryote or eukaryote naturally produces. In some embodiments, the agonist or partial agonist of the mammalian BRS-3 is not material that a prokaryote naturally produces. In some embodiments, the agonist or partial agonist of the mammalian BRS-3 is not material that a eukaryote naturally produces. In some embodiments, the agonist or partial agonist of the mammalian BRS-3 is not material that a mammal naturally produces.

In some embodiments, the agonist or partial agonist of the mammalian BRS-3 is an agonist or partial agonist with an EC₅₀ of less than about 10 μM, of less than about 1 μM, of less than about 100 nM, or of less than about 10 nM at human BRS-3. In some embodiments, the agonist or partial agonist of the mammalian BRS-3 is an agonist or partial agonist with an EC₅₀ of less than a value selected from the interval of about 10 nM to 10 μM. In some embodiments, the agonist or partial agonist of the mammalian BRS-3 is an agonist or partial agonist with an EC₅₀ of less than a value selected from the interval of about 10 nM to 1 μM. In some embodiments, the agonist or partial agonist of the mammalian BRS-3 is an agonist or partial agonist with an EC₅₀ of less than a value selected from the interval of about 10 nM to 100 nM. In some embodiments, the agonist or partial agonist of the mammalian BRS-3 is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the agonist or partial agonist of the mammalian BRS-3 is an agonist or partial agonist with an EC₅₀ of less than about 10 μM, of less than about 1 μM, of less than about 100 nM, or of less than about 10 nM in said assay. In some embodiments, the agonist or partial agonist of the mammalian BRS-3 is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the agonist or partial agonist of the mammalian BRS-3 is an agonist or partial agonist with an EC₅₀ in said assay of less than a value selected from the interval of about 10 nM to 10 μM. In some embodiments, the agonist or partial agonist of the mammalian BRS-3 is an agonist or partial agonist with an EC₅₀ in said assay of less than a value selected from the interval of about 10 nM to 1 μM. In some embodiments, the agonist or partial agonist of the mammalian BRS-3 is an agonist or partial agonist with an EC₅₀ in said assay of less than a value selected from the interval of about 10 nM to 100 nM.

In some embodiments, the agonist or partial agonist of the mammalian BRS-3 is a compound selected from Table D. In some embodiments, the agonist or partial agonist of the mammalian BRS-3 is Compound D28, Compound D30, Compound D31, or Compound D34.

In some embodiments, the agonist or partial agonist of the mammalian BRS-3 is orally active.

In a thirteenth aspect, the invention features use of an agonist or partial agonist of a mammalian BRS-3 for the manufacture of a medicament for promoting wakefulness or for preventing or treating excessive sleepiness, a sleep disorder ameliorated by promoting wakefulness, or a cognitive disorder.

In some embodiments, the use of an agonist or partial agonist of a mammalian BRS-3 for the manufacture of a medicament for promoting wakefulness or for preventing or treating excessive sleepiness, a sleep disorder ameliorated by promoting wakefulness, or a cognitive disorder is a use of an agonist or partial agonist of a mammalian BRS-3 for the manufacture of a medicament for promoting wakefulness or for preventing or treating excessive sleepiness, a sleep disorder ameliorated by promoting wakefulness, or a cognitive disorder in the mammal.

In some embodiments, the mammalian BRS-3 is a human BRS-3. In some embodiments, the mammal is a human.

In some embodiments, the agonist or partial agonist of the mammalian BRS-3 is an agonist or partial agonist of a human BRS-3. In some embodiments, the agonist or partial agonist of the human BRS-3 is an agonist or partial agonist of human BRS-3 having the amino acid sequence of SEQ ID NO: 2. In some embodiments, the agonist or partial agonist of a mammalian or human BRS-3 is a BRS-3 selective agonist or partial agonist.

In some embodiments, the agonist or partial agonist of the mammalian BRS-3 is according to the ninth aspect.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder, wherein the psychological disorder is selected from depression and schizophrenia.

In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the agonist or partial agonist of the mammalian BRS-3 is a small molecule.

In some embodiments, the agonist or partial agonist of the mammalian BRS-3 is a polypeptide. In some embodiments, the agonist or partial agonist of the mammalian BRS-3 is not an antibody or an antigen-binding fragment thereof. In some embodiments, the agonist or partial agonist of the mammalian BRS-3 is a polypeptide, provided that the polypeptide is not an antibody or an antigen-binding fragment thereof. In some embodiments, the agonist or partial agonist of the mammalian BRS-3 is an antibody or an antigen-binding fragment thereof. In some embodiments, the agonist or partial agonist of the mammalian BRS-3 is a lipid. In some embodiments, the agonist or partial agonist of the mammalian BRS-3 is not a polypeptide. In some embodiments, the agonist or partial agonist of the mammalian BRS-3 is not a peptoid. In some embodiments, the agonist or partial agonist of the mammalian BRS-3 is not a lipid. In some embodiments, the agonist or partial agonist of the mammalian BRS-3 is non-endogenous. In some embodiments, the agonist or partial agonist of the mammalian BRS-3 is not material that a prokaryote or eukaryote naturally produces. In some embodiments, the agonist or partial agonist of the mammalian BRS-3 is not material that a prokaryote naturally produces. In some embodiments, the agonist or partial agonist of the mammalian BRS-3 is not material that a eukaryote naturally produces. In some embodiments, the agonist or partial agonist of the mammalian BRS-3 is not material that a mammal naturally produces.

In some embodiments, the agonist or partial agonist of the mammalian BRS-3 is an agonist or partial agonist with an EC₅₀ of less than about 10 μM, of less than about 1 μM, of less than about 100 nM, or of less than about 10 nM at human BRS-3. In some embodiments, the agonist or partial agonist of the mammalian BRS-3 is an agonist or partial agonist with an EC₅₀ of less than a value selected from the interval of about 10 nM to 10 μM. In some embodiments, the agonist or partial agonist of the mammalian BRS-3 is an agonist or partial agonist with an EC₅₀ of less than a value selected from the interval of about 10 nM to 1 μM. In some embodiments, the agonist or partial agonist of the mammalian BRS-3 is an agonist or partial agonist with an EC₅₀ of less than a value selected from the interval of about 10 nM to 100 nM. In some embodiments, the agonist or partial agonist of the mammalian BRS-3 is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the agonist or partial agonist of the mammalian BRS-3 is an agonist or partial agonist with an EC₅₀ of less than about 10 μM, of less than about 1 μM, of less than about 100 nM, or of less than about 10 nM in said assay. In some embodiments, the agonist or partial agonist of the mammalian BRS-3 is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the agonist or partial agonist of the mammalian BRS-3 is an agonist or partial agonist with an EC₅₀ in said assay of less than a value selected from the interval of about 10 μM to 10 μM. In some embodiments, the agonist or partial agonist of the mammalian BRS-3 is an agonist or partial agonist with an EC₅₀ in said assay of less than a value selected from the interval of about 10 μM to 1 μM. In some embodiments, the agonist or partial agonist of the mammalian BRS-3 is an agonist or partial agonist with an EC₅₀ in said assay of less than a value selected from the interval of about 10 nM to 100 nM.

In some embodiments, the agonist or partial agonist of the mammalian BRS-3 is a compound selected from Table D. In some embodiments, the agonist or partial agonist of the mammalian BRS-3 is Compound D28, Compound D30, Compound D31, or Compound D34.

In some embodiments, the agonist or partial agonist of the mammalian BRS-3 is orally active.

In a fourteenth aspect, the invention features an agonist or partial agonist of a mammalian BRS-3 or a pharmaceutical composition comprising the agonist or partial agonist and a pharmaceutically acceptable carrier for use to promote wakefulness or to prevent or treat excessive sleepiness, a sleep disorder ameliorated by promoting wakefulness, or a cognitive disorder.

In some embodiments, the mammalian BRS-3 is a human BRS-3. In some embodiments, the mammal is a human.

In some embodiments, the agonist or partial agonist of a mammalian BRS-3 or a pharmaceutical composition comprising the agonist or partial agonist and a pharmaceutically acceptable carrier for use to promote wakefulness or to prevent or treat excessive sleepiness, a sleep disorder ameliorated by promoting wakefulness, or a cognitive disorder is an agonist or partial agonist of a mammalian BRS-3 for use to promote wakefulness or to prevent or treat excessive sleepiness, a sleep disorder ameliorated by promoting wakefulness, or a cognitive disorder. In some embodiments, the agonist or partial agonist of a mammalian BRS-3 or a pharmaceutical composition comprising the agonist or partial agonist and a pharmaceutically acceptable carrier for use to promote wakefulness or to prevent or treat excessive sleepiness, a sleep disorder ameliorated by promoting wakefulness, or a cognitive disorder is the pharmaceutical composition comprising the agonist or partial agonist of a mammalian BRS-3 and a pharmaceutically acceptable carrier for use to promote wakefulness or to prevent or treat excessive sleepiness, a sleep disorder ameliorated by promoting wakefulness, or a cognitive disorder.

In some embodiments, the agonist or partial agonist of a mammalian BRS-3 or a pharmaceutical composition comprising the agonist or partial agonist and a pharmaceutically acceptable carrier for use to promote wakefulness or to prevent or treat excessive sleepiness, a sleep disorder ameliorated by promoting wakefulness, or a cognitive disorder is an agonist or partial agonist of a mammalian BRS-3 or a pharmaceutical composition comprising the agonist or partial agonist and a pharmaceutically acceptable carrier for use to promote wakefulness or to prevent or treat excessive sleepiness, a sleep disorder ameliorated by promoting wakefulness, or a cognitive disorder in the mammal.

In some embodiments, the agonist or partial agonist of the mammalian BRS-3 is an agonist or partial agonist of a human BRS-3. In some embodiments, the agonist or partial agonist of the human BRS-3 is an agonist or partial agonist of human BRS-3 having the amino acid sequence of SEQ ID NO: 2. In some embodiments, the agonist or partial agonist of a mammalian or human BRS-3 is a BRS-3 selective agonist or partial agonist.

In some embodiments, the agonist or partial agonist of the mammalian BRS-3 is according to the ninth aspect.

In some embodiments, the excessive sleepiness is associated with a sleep disorder.

In some embodiments, the excessive sleepiness is associated with a sleep disorder, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

In some embodiments, the excessive sleepiness is associated with a sleep disorder, wherein the sleep disorder is narcolepsy.

In some embodiments, the excessive sleepiness is associated with a neurological disorder.

In some embodiments, the excessive sleepiness is associated with a neurological disorder, wherein the neurological disorder is selected from the group consisting of multiple sclerosis, myonic dystrophy, and Parkinson's disease.

In some embodiments, the excessive sleepiness is associated with a psychiatric disorder.

In some embodiments, the excessive sleepiness is associated with a psychiatric disorder, wherein the psychological disorder is selected from depression and schizophrenia.

In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic Disorder Not Otherwise Specified. In some embodiments, the Cognitive Disorder is Dementia. In some embodiments, the Cognitive Disorder is Dementia of the Alzheimer's Type.

In some embodiments, the agonist or partial agonist of the mammalian BRS-3 is a small molecule.

In some embodiments, the agonist or partial agonist of the mammalian BRS-3 is a polypeptide. In some embodiments, the agonist or partial agonist of the mammalian BRS-3 is not an antibody or an antigen-binding fragment thereof. In some embodiments, the agonist or partial agonist of the mammalian BRS-3 is a polypeptide, provided that the polypeptide is not an antibody or an antigen-binding fragment thereof. In some embodiments, the agonist or partial agonist of the mammalian BRS-3 is an antibody or an antigen-binding fragment thereof. In some embodiments, the agonist or partial agonist of the mammalian BRS-3 is a lipid. In some embodiments, the agonist or partial agonist of the mammalian BRS-3 is not a polypeptide. In some embodiments, the agonist or partial agonist of the mammalian BRS-3 is not a peptoid. In some embodiments, the agonist or partial agonist of the mammalian BRS-3 is not a lipid. In some embodiments, the agonist or partial agonist of the mammalian BRS-3 is non-endogenous. In some embodiments, the agonist or partial agonist of the mammalian BRS-3 is not material that a prokaryote or eukaryote naturally produces. In some embodiments, the agonist or partial agonist of the mammalian BRS-3 is not material that a prokaryote naturally produces. In some embodiments, the agonist or partial agonist of the mammalian BRS-3 is not material that a eukaryote naturally produces. In some embodiments, the agonist or partial agonist of the mammalian BRS-3 is not material that a mammal naturally produces.

In some embodiments, the agonist or partial agonist of the mammalian BRS-3 is an agonist or partial agonist with an EC₅₀ of less than about 10 μM, of less than about 1 μM, of less than about 100 nM, or of less than about 10 nM at human BRS-3. In some embodiments, the agonist or partial agonist of the mammalian BRS-3 is an agonist or partial agonist with an EC₅₀ of less than a value selected from the interval of about 10 nM to 10 μM. In some embodiments, the agonist or partial agonist of the mammalian BRS-3 is an agonist or partial agonist with an EC₅₀ of less than a value selected from the interval of about 10 nM to 1 μM. In some embodiments, the agonist or partial agonist of the mammalian BRS-3 is an agonist or partial agonist with an EC₅₀ of less than a value selected from the interval of about 10 nM to 100 nM. In some embodiments, the agonist or partial agonist of the mammalian BRS-3 is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the agonist or partial agonist of the mammalian BRS-3 is an agonist or partial agonist with an EC₅₀ of less than about 10 μM, of less than about 1 μM, of less than about 100 nM, or of less than about 10 nM in said assay. In some embodiments, the agonist or partial agonist of the mammalian BRS-3 is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the agonist or partial agonist of the mammalian BRS-3 is an agonist or partial agonist with an EC₅₀ in said assay of less than a value selected from the interval of about 10 nM to 10 μM. In some embodiments, the agonist or partial agonist of the mammalian BRS-3 is an agonist or partial agonist with an EC₅₀ in said assay of less than a value selected from the interval of about 10 nM to 1 μM. In some embodiments, the agonist or partial agonist of the mammalian BRS-3 is an agonist or partial agonist with an EC₅₀ in said assay of less than a value selected from the interval of about 10 nM to 100 nM.

In some embodiments, the agonist or partial agonist of the mammalian BRS-3 is a compound selected from Table D. In some embodiments, the agonist or partial agonist of the mammalian BRS-3 is Compound D28, Compound D30, Compound D31, or Compound D34.

In some embodiments, the agonist or partial agonist of the mammalian BRS-3 is orally active.

In a fifteenth aspect, the invention features a method of screening candidate compounds for a pharmaceutical agent for a GABA-related neurological disorder selected from the group consisting of a sleep disorder ameliorated by promoting sleep, an anxiety disorder, a convulsive disorder, migraine, a depressive disorder, a psychotic disorder, a sleep disorder ameliorated by promoting wakefulness, and a cognitive disorder, said method comprising the elements:
(a) providing a host cell or a membrane of a host cell that comprises a G protein-coupled receptor, said G protein-coupled receptor having at least about 75% identity, at least about 80% identity, at least about 85% identity, at least about 90% identity or at least about 95% identity to SEQ ID NO: 2; and
(b) screening candidate compounds against said G protein-coupled receptor.

In certain embodiments, the method comprises identifying an agonist of the G protein-coupled receptor.

In certain embodiments, the method comprises identifying a partial agonist of the G protein-coupled receptor.

In certain embodiments, the method comprises identifying an inverse agonist of the G protein-coupled receptor.

In certain embodiments, the method comprises identifying an antagonist of the G protein-coupled receptor.

In certain embodiments, said screening comprises determining whether said agonist, partial agonist, inverse agonist or antagonist promotes sleep, has anxiolytic activity, has anticonvulsant activity, has anti-migraine activity, has antidepressant activity, has antipsychotic activity, promotes wakefulness, or has cognition-enhancing activity.

In certain embodiments, said screening comprises the elements:
(i) administering said agonist, partial agonist, inverse agonist or antagonist to a mammal; and
(ii) determining whether said agonist, partial agonist, inverse agonist or antagonist promotes sleep, has anxiolytic activity, has anticonvulsant activity, has anti-migraine activity, has antidepressant activity, has antipsychotic activity, promotes wakefulness, or has cognition-enhancing activity.

In certain embodiments, said mammal is a non-human mammal.

In certain embodiments, the method further comprises formulating said agonist, partial agonist, inverse agonist or antagonist as a pharmaceutical.

In certain embodiments, the G protein-coupled receptor comprises an amino acid sequence having at least about 95% identity to SEQ ID NO: 2.

In certain embodiments, the G protein-coupled receptor comprises the amino acid sequence of SEQ ID NO: 2.

In a sixteenth aspect, the invention features use of a G protein-coupled receptor to screen candidate compounds as pharmaceutical agents for a GABA-related neurological disorder selected from the group consisting of a sleep disorder ameliorated by promoting sleep, an anxiety disorder, a convulsive disorder, migraine, a depressive disorder, a psychotic disorder, a sleep disorder ameliorated by promoting wakefulness, and a cognitive disorder, wherein the G protein-coupled receptor comprises an amino acid sequence having at least about 75% identity, at least about 80% identity, at least about 85% identity, at least about 90% identity or at least about 95% identity to SEQ ID NO: 2.

In certain embodiments, the screen is for an agonist of the G protein-coupled receptor.

In certain embodiments, the screen is for a partial agonist of the G protein-coupled receptor.

In certain embodiments, the screen is for an inverse agonist of the G protein-coupled receptor.

In certain embodiments, the screen is for an antagonist of the G protein-coupled receptor.

In certain embodiments, the G protein-coupled receptor comprises an amino acid sequence having at least about 95% identity to SEQ ID NO: 2.

In certain embodiments, the G protein-coupled receptor comprises the amino acid sequence of SEQ ID NO: 2.

Applicant reserves the right to exclude any one or more candidate compounds from any of the embodiments of the invention. Applicant reserves the right to exclude any one or more modulators from any of the embodiments of the invention. By way of example and not limitation, Applicant reserves the right to exclude any one or more inverse agonists or antagonists from any of the embodiments of the invention. By way of further example and not limitation, Applicant reserves the right to exclude any one or more agonists or partial agonists from any of the embodiments of the invention. Applicant reserves the right to exclude any polynucleotide or polypeptide from any of the embodiments of the invention. Applicant additionally reserves the right to exclude any sleep disorder from any of the embodiments of the invention. Applicant additionally reserves the right to exclude any neurological disorder from any of the embodiments of the invention. Applicant additionally reserves the right to exclude any psychiatric disorder from any of the embodiments of the invention. Applicant additionally reserves the right to exclude any GABA-related neurological disorder from any of the embodiments of the invention. It is also expressly contemplated that sleep disorders of the invention can be included in an embodiment either individually or in any combination. It is also expressly contemplated that neurological disorders of the invention can be included in an embodiment either individually or in any combination. It is also expressly contemplated that psychiatric disorders of the invention can be included in an embodiment either individually or in any combination. It is also expressly contemplated that GABA-related neurological disorders of the invention can be included in embodiments either individually or in any combination.

Without further elaboration, it is believed that one skilled in the art can, using the preceding description, practice the present invention to its fullest extent. The foregoing detailed description is given for clearness of understanding only, and no unnecessary limitation should be understood therefrom, as modifications within the scope of the invention may become apparent to those skilled in the art.

Throughout this application, various publications, patents and published patent applications are cited. The disclosures of these publications, patents and published patent applications referenced in this application are herein incorporated by reference in their entirety into the present disclosure. Citation herein by Applicant of a publication, patent, or published patent application is not an admission by Applicant of said publication, patent, or published patent application as prior art.

This application claims the benefit of priority from the following provisional patent application, filed via U.S. Express mail with the United States Patent and Trademark Office on the indicated date: U.S. Provisional Patent Application No. 60/878,796, filed Jan. 5, 2007. The disclosure of the foregoing provisional patent application is herein incorporated by reference in its entirety.

### BRIEF DESCRIPTION OF THE DRAWINGS

FIG. 1. By way of illustration and not limitation, FIG. 1 depicts results from a primary screen of candidate compounds against a “target receptor” which is a Gsα Fusion Protein construct of an endogenous, constitutively active Gs-coupled GPCR unrelated to BRS-3. Results for “Compound A” are provided in well A2. Results for “Compound “B” are provided in well G9. (See, Example 6.)

FIG. 2. BRS-3 exhibits detectable constitutive activity for increasing intracellular IP3 accumulation. (See, Example 10.)

FIG. 3. Characterization of the agonist activity of [D-Tyr⁶,βAla¹¹,Phe¹³,Nle¹⁴]Bombesin(6-14) at BRS-3. [D-Tyr⁶,βAla¹¹,Phe¹³,Nle¹⁴]Bombesin(6-14) corresponds to Compound D34 in Table D. A. Characterization of the agonist activity of [D-Tyr⁶,βAla¹¹,Phe¹³,Nle¹⁴]Bombesin(6-14) at BRS-3 using FLIPR assay. B. Characterization of the agonist activity of [D-Tyr⁶,βAla¹¹,Phe¹³,Nle¹⁴]Bombesin(6-14) at BRS-3 using melanophore assay. C. Characterization of the agonist activity of [D-Tyr⁶,βAla¹¹,Phe¹³,Nle¹⁴]Bombesin(6-14) at BRS-3 using IP3 accumulation assay. (See, Example 11.)

FIG. 4. Representative photomicrographic images illustrating the expression of BRS-3 and GAD67 in the dorsomedial hypothalamic nucleus (DMH) in rat are presented as panels A to C. (See, Example 14.)

FIG. 5. A representative graphic presentation of the expression of BRS-3 and GAD67 in the dorsomedial hypothalamic nucleus (DMH) in rat, where FIG. 5B is an enlargement of part of FIG. 5A. (See, Example 14.)

FIG. 6. Results of in situ hybridization analysis of the co-expression of BRS-3 and a number of neurotransmitters or markers by neurons within subregions of the hypothalamus exhibiting detectable expression of BRS-3. (See, Example 14.)

### DETAILED DESCRIPTION

### Definitions

AGONIST shall mean an agent (e.g., ligand, candidate compound) that by virtue of binding to a GPCR activates the GPCR so as to elicit an intracellular response mediated by the GPCR.

AMINO ACID ABBREVIATIONS used herein are set out in Table B:

TABLE B

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

ANTAGONIST shall mean an agent (e.g., ligand, candidate compound) that binds, and preferably binds competitively, to a GPCR at about the same site as an agonist or partial agonist but which does not activate an intracellular response initiated by the active form of the GPCR, and can thereby inhibit the intracellular response by agonist or partial agonist. An antagonist typically does not diminish the baseline intracellular response in the absence of an agonist or partial agonist.

ANTIBODY is intended herein to encompass monoclonal antibody and polyclonal antibody. Antibodies of the present invention may be prepared by any suitable method known in the art.

ANXIETY DISORDER shall be understood to include disorders associated with excessive anxiety. Anxiety Disorders shall be understood to include but not necessarily be limited to Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified, as these terms are defined in DSM-IV-TR® (American Psychiatric Association, “DSM-IV-TR®” (Diagnositic and Statistical Manual of Mental Disorders, Fourth Edition, Text Revision), 2000).

BIOLOGICALLY ACTIVE FRAGMENT of a GPCR polypeptide or amino acid sequence shall mean a fragment of the polypeptide or amino acid sequence having structural and biochemical functions of a naturally occurring GPCR. In certain embodiments, the biologically active fragment couples to a G protein. In certain embodiments, the biologically active fragment binds to a ligand.

CANDIDATE COMPOUND shall mean a molecule (for example, and not limitation, a chemical compound) that is amenable to a screening technique and is used interchangeably herein with TEST COMPOUND.

CATAPLEXY shall refer to episodes of sudden loss of muscle tone, usually triggered by emotion.

CODON shall mean a grouping of three nucleotides (or equivalents to nucleotides) which generally comprise a nucleoside [adenosine (A), guanosine (G), cytidine (C), uridine (U) and thymidine (T)] coupled to a phosphate group and which, when translated, encodes an amino acid.

COGNITIVE DISORDER shall be understood to include disorders that have a significant impairment of cognition as compared to a previous level of functioning as a predominant feature. Cognitive Disorders shall be understood to include but not necessarily be limited to Delirium (including but not necessarily limited to Delirium Due to a General Medical Condition, Substance-Induced Delirium, Delirium Due to Multiple Etiologies, and Delirium Not Otherwise Specified), Dementia (including but not necessarily limited to Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified), Amnestic Disorders (including but not necessarily limited to Amnestic Disorder Due to a General Medical Condition, Substance-Induced Persisting Amnestic Disorder, and Amnestic Disorder Not Otherwise Specified), and Cognitive Disorder Not Otherwise Specified, as these terms are defined in DSM-IV-TR® (American Psychiatric Association, “DSM-IV-TR®” (Diagnositic and Statistical Manual of Mental Disorders, Fourth Edition, Text Revision), 2000).

COMPOSITION means a material comprising at least one component.

COMPOUND EFFICACY or EFFICACY shall mean the ability of a compound to inhibit or stimulate one or more GPCR functions, e.g. by measurement of cAMP level in the presence or absence of a candidate compound. Exemplary means of measuring compound efficacy are disclosed in the Examples section of this patent document.

CONSTITUTIVELY ACTIVE RECEPTOR shall mean a receptor stabilized in an active state by means other than through binding of the receptor to its ligand or a chemical equivalent thereof. A constitutively active receptor may be endogenous or non-endogenous.

CONSTITUTIVELY ACTIVATED RECEPTOR shall mean an endogenous receptor that has been modified so as to be constitutively active or to be more constitutively active.

CONSTITUTIVE RECEPTOR ACTIVATION shall mean activation of a receptor in the absence of binding to its ligand or a chemical equivalent thereof.

CONTACT or CONTACTING shall mean bringing at least two moieties together, whether in an in vitro system or an in vivo system.

CONVULSIVE DISORDER shall be understood to include disorders of a subject in which the subject suffers from convulsions, e.g., convulsions due to epileptic seizure. Convulsive Disorders include, but are not necessarily limited to, epilepsy and non-epileptic seizures, e.g., convulsions due to administration of a convulsive agent to the subject.

DEPRESSIVE DISORDER shall be understood to include but not necessarily be limited to Major Depressive Disorder, Dysthymic Disorder, and Depressive Disorder Not Otherwise Specified, as these terms are defined in DSM-1V-TR® (American Psychiatric Association, “DSM-IV-TR®” (Diagnositic and Statistical Manual of Mental Disorders, Fourth Edition, Text Revision), 2000).

DIRECTLY IDENTIFYING or DIRECTLY IDENTIFIED, in relationship to the phrase “candidate compound” or “test compound”, shall mean the screening of a compound against a G protein-coupled receptor in the absence of a known ligand (e.g., a known agonist) to the G protein-coupled receptor.

DYSSOMNIA shall refer to a disorder of initiating or maintaining sleep. ENDOGENOUS shall mean a material that a mammal naturally produces. Endogenous in reference to, for example and not limitation, the term “receptor,” shall mean that which is naturally produced by a mammal (for example, and not limitation, a human). Endogenous shall be understood to encompass allelic variants of a gene as well as the allelic polypeptide variants so encoded. As used herein, “endogenous GPCR” and “native GPCR” are used interchangeably. By contrast, the term NON-ENDOGENOUS in this context shall mean that which is not naturally produced by a mammal (for example, and not limitation, a human).

EXPRESSION VECTOR shall mean a DNA sequence that is required for the transcription of cloned DNA and translation of the transcribed mRNA in an appropriate host cell recombinant for the expression vector. An appropriately constructed expression vector should contain an origin of replication for autonomous replication in host cells, selectable markers, a limited number of useful restriction enzyme sites, a potential for high copy number, and active promoters. The cloned DNA to be transcribed is operably linked to a constitutively or conditionally active promoter within the expression vector.

G PROTEIN-COUPLED RECEPTOR FUSION PROTEIN and GPCR FUSION PROTEIN, in the context of the invention disclosed herein, each mean a non-endogenous protein comprising an endogenous, constitutively active GPCR or a non-endogenous, constitutively activated GPCR fused to at least one G protein, most preferably the alpha (α) subunit of such G protein (this being the subunit that binds GTP), with the G protein preferably being of the same type as the G protein that naturally couples with endogenous GPCR. In the preferred form, the G protein can be fused directly to the C-terminus of the GPCR or there may be spacers between the two.

HOST CELL shall mean a cell capable of having a vector incorporated therein. In the present context, the vector will typically contain nucleic acid encoding a GPCR or GPCR fusion protein in operable connection with a suitable promoter sequence to permit expression of the GPCR or GPCR fusion protein to occur.

HYPERSOMNIA shall refer to excessive daytime sleepiness (EDS).

IN NEED OF PREVENTION OR TREATMENT as used herein refers to a judgement made by a caregiver (e.g. physician, nurse, nurse practitioner, etc. in the case of humans; veterinarian in the case of animals, including non-human mammals) that a subject or animal requires or will benefit from treatment. This judgement is made based on a variety of factors that are in the realm of a caregiver's expertise, but that include the knowledge that the subject or animal is ill, or will be ill, as the result of a condition that is treatable by the compounds of the invention.

INHIBIT or INHIBITING, in relationship to the term “response” shall mean that a response is decreased or prevented in the presence of a compound as opposed to in the absence of the compound.

INSOMNIA shall refer to the perception of inadequate or non-restful sleep by a subject.

INVERSE AGONIST shall mean an agent (e.g., ligand, candidate compound) which binds to a GPCR and which inhibits the baseline intracellular response initiated by the active form of the receptor below the normal base level activity which is observed in the absence of an agonist or partial agonist.

LIGAND as used herein shall mean a molecule that specifically binds to a GPCR. An endogenous ligand is an endogenous molecule that binds to a native GPCR. A ligand of a GPCR may be, but is not limited to, an agonist, a partial agonist, an inverse agonist or an antagonist of the GPCR.

MIGRAINE shall be understood to refer to a painful headache which, in some cases, is preceded or accompanied by a sensory warning sign (aura), such as flashes of light, blind spots or tingling in the arm or leg. A Migraine is also often accompanied by other signs and symptoms, such as nausea, vomiting, and extreme sensitivity of light and sound.

As used herein, the terms MODULATE or MODIFY are meant to refer to an increase or decrease in the amount, quality, or effect of a particular activity, function or molecule.

MODULATOR shall be understood to encompass agonist, partial agonist, inverse agonist and antagonist as hereinbefore defined.

NARCOLEPSY shall refer to a sleep disorder characterized by excessive daytime sleepiness (EDS), disturbed nocturnal sleep, abnormal rapid eye movement (REM) sleep and, frequently, cataplexy.

PARASOMNIA shall refer to behavioral disturbance related to sleep.

PARTIAL AGONIST shall mean an agent (e.g., ligand, candidate compound) that by virtue of binding to a GPCR activates the GPCR so as to elicit an intracellular response mediated by the GPCR, albeit to a lesser extent or degree than does a full agonist.

PHARMACEUTICAL COMPOSITION shall mean a composition comprising at least one active ingredient, whereby the composition is amenable to investigation for a specified, efficacious outcome in a mammal (for example, and not limited to a human). Those of ordinary skill in the art will understand and appreciate the techniques appropriate for determining whether an active ingredient has a desired efficacious outcome e.g., based upon the needs of the artisan.

POLYNUCLEOTIDE shall refer to RNA, DNA, or RNA/DNA hybrid sequence of more than one nucleotide in either single chain or duplex form. The polynucleotides of the invention may be prepared by any known method, including synthetic, recombinant, ex vivo generation, or a combination thereof, as well as utilizing any purification methods known in the art.

POLYPEPTIDE shall refer to a polymer of amino acids without regard to the length of the polymer. Thus, PEPTIDES, oligopeptides, and proteins are included within the definition of polypeptide. This term also does not specify or exclude post-expression modifications of polypeptides. For example, polypeptides that include the covalent attachment of glycosyl groups, acetyl groups, phosphate groups, lipid groups and the like are expressly encompassed by the term polypeptide.

PRIMER is used herein to denote a specific oligonucleotide sequence which is complementary to a target nucleotide sequence and used to hybridize to the target nucleotide sequence. A primer serves as an initiation point for nucleotide polymerization catalyzed by DNA polymerase, RNA polymerase, or reverse transcriptase.

PROMOTION OF SLEEP shall refer to an increase in the duration and/or quality of sleep.

PROMOTION OF WAKEFULNESS shall refer to an increase in the duration of wakefulness.

PSYCHOTIC DISORDER shall be understood to include but not necessarily be limited to Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified, as these terms are defined in DSM-IV-TR® (American Psychiatric Association, “DSM-IV-TR®” (Diagnositic and Statistical Manual of Mental Disorders, Fourth Edition, Text Revision), 2000).

RECEPTOR FUNCTIONALITY shall refer to the normal operation of a receptor to receive a stimulus and moderate an effect in the cell, including, but not limited to regulating gene transcription, regulating the influx or efflux of ions, effecting a catalytic reaction, and/or modulating activity through G-proteins, such as eliciting a second messenger response.

SCHIZOPHRENIA shall be understood to include but not necessarily be limited to the subtypes Paranoid, Disorganized, Catatonic, Undifferentiated, and Residual, as these terms are defined in DSM-IV-TR® (American Psychiatric Association, “DSM-1V-TR®” (Diagnositic and Statistical Manual of Mental Disorders, Fourth Edition, Text Revision), 2000).

SECOND MESSENGER shall mean an intracellular response produced as a result of receptor activation. A second messenger can include, for example, inositol 1,4,5-triphosphate (IP3), diacylglycerol (DAG), cyclic AMP (cAMP), cyclic GMP (cGMP), MAP kinase activity, MAPK/ERK kinase kinase-1 (MEKK1) activity, and Ca²⁺. Second messenger response can be measured for a determination of receptor activation. In addition, second messenger response can be measured for the identification of candidate compounds as, for example, inverse agonists, partial agonists, agonists, and antagonists of the receptor.

SELECTIVE BRS-3 MODULATOR, as used herein, refers to a modulator of BRS-3 having selectivity for BRS-3 receptor over one or more closely related receptors, such as gastric-releasing peptide receptor (GRP-R) or neuromedin B receptor (NMB-R).

SLEEP DISORDER shall refer to disturbance of usual sleep patterns or behaviors. Sleep Disorders shall be understood to include but not necessarily be limited to Sleep Disorders set forth in: American Academy of Sleep Medicine, ICSD—International classification of sleep disorders, revised: Diagnostic and coding manual, American Academy of Sleep Medicine, 2001.

SMALL MOLECULE shall be taken to mean a compound having a molecular weight of less than about 10,000 grams per mole, including a peptide, peptidomimetic, amino acid, amino acid analogue, polynucleotide, polynucleotide analogue, nucleotide, nucleotide analogue, organic compound or inorganic compound (i.e. including a heterorganic compound or organometallic compound), and salts, esters and other pharmaceutically acceptable forms thereof. In certain preferred embodiments, small molecules are organic or inorganic compounds having a molecular weight of less than about 5,000 grams per mole. In certain preferred embodiments, small molecules are organic or inorganic compounds having molecular weight of less than about 1,000 grams per mole. In certain preferred embodiments, small molecules are organic or inorganic compounds having a molecular weight of less than about 500 grams per mole.

STIMULATE or STIMULATING, in relationship to the term “response” shall mean that a response is increased in the presence of a compound as opposed to in the absence of the compound.

SUBJECT as used herein shall preferably refer to a mammal, including but not limited to a mouse, a rat, a rabbit, a pig, a dog, a cat, a non-human primate, a non-human mammal and a human, more preferably to a mouse or rat, most preferably to a human.

THERAPEUTICALLY EFFECTIVE AMOUNT as used herein refers to the amount of active compound or pharmaceutical agent that elicits the biological or medicinal response in a tissue, system, animal, subject or human that is being sought by a researcher, veterinarian, medical doctor or other clinician, which includes one or more of the following:
(1) Preventing the disease; for example, preventing a disease, condition or disorder in a subject that may be predisposed to the disease, condition or disorder but does not yet experience or display the pathology or symptomatology of the disease,
(2) Inhibiting the disease; for example, inhibiting a disease, condition or disorder in a subject that is experiencing or displaying the pathology or symptomatology of the disease, condition or disorder (i.e., arresting further development of the pathology and/or symptomatology), and
(3) Ameliorating the disease; for example, ameliorating a disease, condition or disorder in a subject that is experiencing or displaying the pathology or symptomatology of the disease, condition or disorder (i.e., reversing the pathology and/or symptomatology).

VARIANT as the term is used herein, is a polynucleotide or polypeptide that differs from a reference polynucleotide or polypeptide respectively, but which may retain one or more essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. A typical variant of a polypeptide differs in amino acid sequence from another, reference polypeptide. A variant and reference polypeptide may differ in amino acid sequence by one or more substitutions, additions, deletions in any combination. A variant of a polynucleotide or polypeptide may be a naturally occurring one such as an ALLELIC VARIANT, or it may be a variant that is not known to occur naturally. Non-naturally occurring variants of polynucleotides and polypeptides may be made by mutagenesis techniques or by direct synthesis.

### A. Introduction

The order of the following sections is set forth for presentational efficiency and is not intended, nor should be construed, as a limitation on the disclosure or the claims to follow.

### B. Receptor Expression

1. GPCR Polypeptides of Interest

A GPCR of the invention may comprise an amino acid sequence selected from the group consisting of:
(a) the amino acid sequence of SEQ ID NO: 2;
(b) amino acids 2-399 of SEQ ID NO: 2;
(c) the amino acid sequence of a G protein-coupled receptor encoded by a polynucleotide that is amplifiable by polymerase chain reaction (PCR) on a human DNA sample using specific primers SEQ ID NO: 3 and SEQ ID NO: 4;
(d) the amino acid sequence of a G protein-coupled receptor encoded by a polynucleotide that hybridizes under stringent conditions to the complement of SEQ ID NO: 1;
(e) the amino acid sequence of a G protein-coupled receptor having an amino acid sequence derived from SEQ ID NO: 2 by substitution, deletion or addition of one or several amino acids in the amino acid sequence of SEQ ID NO: 2;
(f) the amino acid sequence of a G protein-coupled receptor having at least about 75%, at least about 80%, at least about 85%, at least about 90% or at least about 95% identity to SEQ ID NO: 2;
(g) the amino acid sequence of a G protein-coupled receptor that is a constitutively active version of a receptor having SEQ ID NO: 2; and
(h) a biologically active fragment of any one of (a) to (g).

In some embodiments, the GPCR comprises the amino acid sequence of SEQ ID NO: 2.

In some embodiments, the GPCR comprises the amino acid sequence of a G protein-coupled receptor having at least about 75%, at least about 80%, at least about 85%, at least about 90% or at least about 95% identity to SEQ ID NO: 2.

In some embodiments, the G protein-coupled receptor having at least about 75%, at least about 80%, at least about 85%, at least about 90% or at least about 95% identity to SEQ ID NO: 2 is an endogenous G protein-coupled receptor. In some embodiments, the G protein-coupled receptor having at least about 75%, at least about 80%, at least about 85%, at least about 90% or at least about 95% identity to SEQ ID NO: 2 is an endogenous mammalian G protein-coupled receptor. In some embodiments, the G protein-coupled receptor having at least about 75%, at least about 80%, at least about 85%, at least about 90% or at least about 95% identity to SEQ ID NO: 2 is a non-endogenous G protein-coupled receptor.

In some embodiments, the G protein-coupled receptor that is a constitutively active version of a receptor having SEQ ID NO: 2 is an endogenous G protein-coupled receptor. In some embodiments, the G protein-coupled receptor that is a constitutively active version of a receptor having SEQ ID NO: 2 is an endogenous G protein-coupled receptor having SEQ ID NO: 2. In some embodiments, the G protein-coupled receptor that is a constitutively active version of a receptor having SEQ ID NO: 2 is a non-endogenous G protein-coupled receptor.

In some embodiments, the human DNA is human cDNA derived from a tissue or cell type that expresses BRS-3. In some embodiments, the human cDNA is derived from hypothalamus.

In some embodiments, a GPCR of the invention is recombinant. In some embodiments, the recombinant GPCR is a mammalian BRS-3. In some embodiments, the recombinant GPCR is a human BRS-3.

In some embodiments, a GPCR of the invention is endogenous.

In some embodiments, a GPCR of the invention is non-endogenous.

In some embodiments, a GPCR of the invention is a mammalian BRS-3.

In some embodiments, a GPCR of the invention that is endogenous is a mammalian BRS-3.

In some embodiments, a GPCR of the invention is constitutively active. In some embodiments, a GPCR of the invention that is constitutively active is an endogenous GPCR. In some embodiments, a GPCR of the invention that is constitutively active is a non-endogenous GPCR. In some embodiments, a GPCR of the invention that is constitutively active is a mammalian BRS-3. In some embodiments, the mammalian BRS-3 is human BRS-3. In some embodiments, the human BRS-3 is SEQ ID NO: 2 or an allele thereof.

In some embodiments, an endogenous GPCR of the invention is constitutively active. In some embodiments, a non-endogenous GPCR of the invention is constitutively active. In some embodiments, a mammalian BRS-3 of the invention is constitutively active. In some embodiments, the mammalian BRS-3 is human BRS-3. In some embodiments, the human BRS-3 is SEQ ID NO: 2 or an allele thereof.

In some embodiments, a GPCR of the invention exhibits a detectable level of constitutive activity. In some embodiments, an endogenous GPCR of the invention exhibits a detectable level of constitutive activity. In some embodiments, a non-endogenous GPCR of the invention exhibits a detectable level of constitutive activity. In some embodiments, a mammalian BRS-3 of the invention exhibits a detectable level of constitutive activity. In some embodiments, the mammalian BRS-3 is human BRS-3. In some embodiments, the human BRS-3 is SEQ ID NO: 2 or an allele thereof.

In some embodiments, a G protein-coupled receptor that may be used in the subject methods is a constitutively active version of a receptor having SEQ ID NO: 2. In some embodiments, the constitutively active version of a receptor having SEQ ID NO: 2 is an endogenous G protein-coupled receptor. In some embodiments, the constitutively active version of a receptor having SEQ ID NO: 2 is an endogenous G protein-coupled receptor having SEQ ID NO: 2. In some embodiments, the constitutively active version of a receptor having SEQ ID NO: 2 is a non-endogenous G protein-coupled receptor. In some embodiments, the constitutive activity is for increasing intracellular IP3. In some embodiments, the constitutive activity is for causing melanophore cells to undergo pigment dispersion. In certain embodiments, the constitutively active version of a receptor having SEQ ID NO: 2 is a G protein-coupled receptor for which [D-Tyr⁶,βAla¹¹,Phe¹³,Nle¹⁴]Bombesin(6-14) is an agonist having an EC₅₀ value at said receptor in FLIPR assay or in melanophore assay or in IP3 assay of less than about 10 μM, less than about 5 μM, less than about 1 μM, less than about 100 nM, less than about 50 nM, less than about 25 nM, less than about 10 nM, or less than about 5 nM.

By way of illustration and not limitation, deletion of an N-terminal methionine residue or an N-terminal signal peptide is envisioned to provide a biologically active fragment that may be used in the subject invention. In some embodiments, a biologically active fragment of the invention is a fragment that exhibits a detectable level of constitutive activity. In some embodiments, the constitutive activity is for increasing intracellular IP3. In some embodiments, the constitutive activity is for causing melanophore cells to undergo pigment dispersion. In certain embodiments, a biologically active fragment of the invention is a G protein-coupled receptor for which β-Tyr⁶,βAla¹¹,Phe¹³,Nle¹⁴]Bombesin(6-14) is an agonist having an EC₅₀ value at said receptor in FLIPR assay or in melanophore assay or in IP3 assay of less than about 10 μM, less than about 5 μM, less than about 1 μM, less than about 100 nM, less than about 50 nM, less than about 25 nM, less than about 10 nM, or less than about 5 nM. In certain embodiments, a biologically active fragment of the invention is a fragment that specifically binds an antibody to mammalian BRS-3. Antibodies to BRS-3 are commercially available; for example, antibody to human BRS-3 is available from Atlas Antibodies (Stockholm, Sweden) and from ABR-Affinity BioReagents (Golden, Colo.).

An allelic variant of human BRS-3 of SEQ ID NO: 2 is envisioned to be within the scope of the invention.

A variant which is a mammalian ortholog of human BRS-3 of SEQ ID NO: 2 is envisioned to be within the scope of the invention. By way of illustration and not limitation, mouse BRS-3 (e.g., GenBank® Accession No. NP_{—}033896), rat BRS-3 (e.g., GenBank® Accession No. AF510984), chimpanzee BRS-3 (e.g., GenBank® Accession No. XP_{—}001137541), rheusus monkey BRS-3 (e.g., GenBank® Accession No. NP_{—}001028074), dog BRS-3 (e.g., GenBank® Accession No. XP_{—}854769), sheep BRS-3 (e.g., GenBank® Accession No. NP_{—}001009215), cow BRS-3 (e.g., GenBank® Accession No. XP_{—}584211), and guinea pig BRS-3 (e.g., GenBank® Accession No. P35371) are envisioned to be within the scope of the invention.

In certain embodiments, a variant G protein-coupled receptor that may be used in the subject methods is a G protein coupled receptor having an amino acid sequence derived from SEQ ID NO: 2 by substitution, deletion or addition of one or several amino acids in the amino acid sequence of SEQ ID NO: 2. In some embodiments, the variant is an endogenous G protein-coupled receptor. In some embodiments, the variant is an endogenous mammalian G protein-coupled receptor. In some embodiments, the variant is an endogenous human G protein-coupled receptor. In some embodiments, the variant is a non-endogenous G protein-coupled receptor. In some embodiments, the variant exhibits a detectable level of constitutive activity. In some embodiments, the constitutive activity is for increasing intracellular IP3. In some embodiments, the constitutive activity is for causing melanophore cells to undergo pigment dispersion. In certain embodiments, said G protein-coupled receptor having an amino acid sequence derived from SEQ ID NO: 2 is a G protein-coupled receptor for which [D-Tyr⁶,βAla¹¹,Phe¹³,Nle¹⁴]Bombesin(6-14) is an agonist having an EC₅₀ value at said receptor in FLIPR assay or in melanophore assay or in IP3 assay of less than about 10 μM, less than about 5 μM, less than about 1 μM, less than about 100 nM, less than about 50 nM, less than about 25 nM, less than about 10 nM, or less than about 5 nM.

In certain embodiments, a variant G protein-coupled receptor that may be used in the subject methods is a G protein-coupled receptor having an amino acid sequence derived from SEQ ID NO: 2 by no more than 10 conservative amino acid substitutions and/or no more than 3 non-conservative amino acid substitutions in the amino acid sequence of SEQ ID NO: 2. In certain embodiments, arginine, lysine and histidine may conservatively substitute for each other; glutamic acid and aspartic acid may conservatively substitute for each other; glutamine and asparagine may conservatively substitute for each other; leucine, isoleucine and valine may conservatively substitute for each other; phenylalanine, tryptophan and tyrosine may conservatively substitute for each other; and glycine, alanine, serine, threonine and methionine may conservatively substitute for each other. The amino acid substitutions, amino acid deletions, and amino acid additions may be at any position (e.g., the C- or N-terminus, or at internal positions). In some embodiments, the variant is an endogenous G protein-coupled receptor. In some embodiments, the variant is an endogenous mammalian G protein-coupled receptor. In some embodiments, the variant is an endogenous human G protein-coupled receptor. In some embodiments, the variant is a non-endogenous G protein-coupled receptor. In some embodiments, the variant exhibits a detectable level of constitutive activity. In some embodiments, the constitutive activity is for increasing intracellular IP3. In some embodiments, the constitutive activity is for causing melanophore cells to undergo pigment dispersion. In certain embodiments, said G protein-coupled receptor having an amino acid sequence derived from SEQ ID NO: 2 is a G protein-coupled receptor for which [D-Tyr⁶,βAla¹¹,Phe¹³,Nle¹⁴]Bombesin(6-14) is an agonist having an EC₅₀ value at said receptor in FLIPR assay or in melanophore assay or in IP3 assay of less than about 10 μM, less than about 5 μM, less than about 1 μM, less than about 100 nM, less than about 50 nM, less than about 25 nM, less than about 10 nM, or less than about 5 nM.

A variant G protein-coupled receptor having at least about 75%, at least about 80%, at least about 85%, at least about 90%, at least about 95%, at least about 96%, at least about 97%, at least about 98% or at least about 99% identity to SEQ ID NO: 2 is envisioned to be within the scope of the invention. In some embodiments, the variant is an endogenous G protein-coupled receptor. In some embodiments, the variant is an endogenous mammalian G protein-coupled receptor. In some embodiments, the variant is an endogenous human G protein-coupled receptor. In some embodiments, the variant is a non-endogenous G protein-coupled receptor. In some embodiments, the variant exhibits a detectable level of constitutive activity. In some embodiments, the constitutive activity is for increasing intracellular IP3. In some embodiments, the constitutive activity is for causing melanophore cells to undergo pigment dispersion. In certain embodiments, the G protein-coupled receptor having at least about 75%, at least about 80%, at least about 85%, at least about 90%, at least about 95%, at least about 96%, at least about 97%, at least about 98% or at least about 99% identity to SEQ ID NO: 2 is a G protein-coupled receptor for which [D-Tyr⁶,βAla¹¹,Phe¹³,Nle¹⁴]Bombesin(6-14) is an agonist having an EC₅₀ value at said receptor in FLIPR assay or in melanophore assay or in IP3 assay of less than about 10 μM, less than about 5 μM, less than about 1 μM, less than about 100 nM, less than about 50 nM, less than about 25 nM, less than about 10 nM, or less than about 5 nM. In certain embodiments, the G protein-coupled receptor having at least about 75%, at least about 80%, at least about 85%, at least about 90%, at least about 95%, at least about 96%, at least about 97%, at least about 98% or at least about 99% identity to SEQ ID NO: 2 is a G protein-coupled receptor that specifically binds an antibody to mammalian BRS-3. Antibodies to BRS-3 are commercially available; for example, antibody to human BRS-3 is available from Atlas Antibodies (Stockholm, Sweden) and from ABR-Affinity BioReagents (Golden, Colo.). Percent identity can be determined conventionally using known computer programs.

In certain embodiments, a variant G protein-coupled receptor that may be used in the subject methods has an amino acid sequence having at least about 75%, at least about 80%, at least about 85%, at least about 90%, or at least about 95% identity to SEQ ID NO: 2. By a variant G protein-coupled receptor having, for example, 95% “identity” to SEQ ID NO: 2 is meant that the amino acid sequence of the variant is identical to amino acids 1-399 of SEQ ID NO: 2 except that it may include up to five amino acid alterations per each 100 amino acids of SEQ ID NO: 2. Thus, to obtain for example an amino acid sequence having at least 95% identity to the amino acid sequence of SEQ ID NO: 2, up to 5% (5 of 100) of the amino acid residues in the sequence may be inserted, deleted, or substituted with another amino acid compared with amino acids 1-399 of SEQ ID NO: 2. These alternations may occur at the amino or carboxy termini or anywhere between those terminal positions, interspersed either subjectly among residues in the sequence or in one or more contiguous groups within the sequence.

In some embodiments, a variant G protein-coupled receptor that may be used in the subject methods is a G protein-coupled receptor encoded by a polynucleotide that hybridizes under stringent conditions to the complement of SEQ ID NO: 1. In some embodiments, the variant is an endogenous G protein-coupled receptor. In some embodiments, the variant is an endogenous mammalian G protein-coupled receptor. In some embodiments, the variant is an endogenous human G protein-coupled receptor. In some embodiments, the variant is a non-endogenous G protein-coupled receptor. In some embodiments, the variant exhibits a detectable level of constitutive activity. In some embodiments, the constitutive activity is for increasing intracellular IP3. In some embodiments, the constitutive activity is for causing melanophore cells to undergo pigment dispersion. In certain embodiments, the G protein-coupled receptor encoded by a polynucleotide that hybridizes under stringent conditions to the complement of SEQ ID NO: 1 is a G protein-coupled receptor for which [D-Tyr⁶,βAla¹¹,Phe¹³,Nle¹⁴]Bombesin(6-14) is an agonist having an EC₅₀ value at said receptor in FLIPR assay or in melanophore assay or in IP3 assay of less than about 10 μM, less than about 5 μM, less than about less than about 100 nM, less than about 50 nM, less than about 25 nM, less than about 10 nM, or less than about 5 nM. Hybridization techniques are well known to the skilled artisan. In some embodiments, stringent hybridization conditions include overnight incubation at 42° C. in a solution comprising: 50% formamide, 5×SSC (1×SSC=150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA; followed by washing the filter in 0.1×SSC or in 0.2×SSC at about 50° C., at about 55° C., at about 60° C. or at about 65° C. In some embodiments, stringent hybridization conditions include overnight incubation at 42° C. in a solution comprising: 50% formamide, 5×SSC (1×SSC=150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA; followed by washing the filter in 0.1×SSC/0.1% SDS (sodium dodecyl sulfate) or in 0.2×SSC/0.1% SDS at about 50° C., at about 55° C., at about 60° C. or at about 65° C.

a. Sequence Identity

In certain embodiments, percent identity is evaluated using the Basic Local Alignment Search Tool (“BLAST”), which is well known in the art [See, e.g., Karlin and Altschul, Proc Natl Acad Sci USA (1990) 87:2264-2268; Altschul et al., J Mol Biol (1990) 215:403-410; Altschul et all, Nature Genetics (1993) 3:266-272; and Altschul et al., Nucleic Acids Res (1997) 25:3389-3402; the disclosure of each of which is herein incorporated by reference in its entirety]. The BLAST programs may be used with the default parameters or with modified parameters provided by the user. Preferably, the parameters are default parameters.

In certain embodiments, a preferred method for determining the best overall match between a query sequence (e.g., the amino acid sequence of SEQ ID NO:2) and a sequence to be interrogated, also referred to as a global sequence alignment, can be determined using the FASTDB computer program based on the algorithm of Brutlag et al. [Comp App Biosci (1990) 6:237-245; the disclosure of which is herein incorporated by reference in its entirety]. In a sequence alignment the query and interrogated sequences are both amino acid sequences. The results of said global sequence alignment is in percent identity. Preferred parameters used in a FASTDB amino acid alignment are: Matrix=PAM 0, k-tuple=2, Mismatch Penalty=1, Joining Penalty=20, Randomization Group=25, Length=O, Cutoff Score=1, Window Size=sequence length, Gap Penalty=5, Gap Size Penalty=0.05, Window Size=247 or the length of the interrogated amino acid sequence, whichever is shorter.

If the interrogated sequence is shorter than the query sequence due to N- or C-terminal deletions, not because of internal deletions, the results, in percent identity, must be manually corrected because the FASTDB program does not account for N- and C-terminal truncations of the interrogated sequence when calculating global percent identity. For interrogated sequences truncated at the N- and C-termini, relative to the query sequence, the percent identity is corrected by calculating the number of residues of the query sequence that are N- and C-terminal of the interrogated sequence, that are not matched/aligned with a corresponding interrogated sequence residue, as a percent of the total residues of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the interrogated sequence, which are not matched/aligned with the query sequence, are considered for the purposes of manually adjusting the percent identity score. That is, only query amino acid residues outside the farthest N- and C-terminal residues of the interrogated sequence.

For example, a 90 amino acid residue interrogated sequence is aligned with a 100-residue query sequence to determine percent identity. The deletion occurs at the N-terminus of the interrogated sequence and therefore, the FASTDB alignment does not match/align with the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched, the final percent identity would be 90%.

In another example, a 90-residue interrogated sequence is compared with a 100-residue query sequence. This time the deletions are internal so there are no residues at the N- or C-termini of the interrogated sequence, which are not matched/aligned with the query. In this case, the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other corrections are made for the purposes of the present invention.

b. Fusion Proteins

In certain embodiments, a polypeptide of interest is a fusion protein, and may contain, for example, an affinity tag domain or a reporter domain. Suitable affinity tags include any amino acid sequence that may be specifically bound to another moiety, usually another polypeptide, most usually an antibody. Suitable affinity tags include epitope tags, for example, the V5 tag, the FLAG tag, the HA tag (from hemagglutinin influenza virus), the myc tag, and the like, as is known in the art. Suitable affinity tags also include domains for which, binding substrates are known, e.g., HIS, GST and MBP tags, as is known in the art, and domains from other proteins for which specific binding partners, e.g., antibodies, particularly monoclonal antibodies, are available. Suitable affinity tags also include any protein-protein interaction domain, such as a IgG Fc region, which may be specifically bound and detected using a suitable binding partner, e.g. the IgG Fc receptor. It is expressly contemplated that such a fusion protein may contain a heterologous N-terminal domain (e.g., an epitope tag) fused in-frame with a GPCR that has had its N-terminal methionine residue either deleted or substituted with an alternative amino acid.

Suitable reporter domains include any domain that can report the presence of a polypeptide. While it is recognized that an affinity tag may be used to report the presence of a polypeptide using, e.g., a labeled antibody that specifically binds to the tag, light emitting reporter domains are more usually used. Suitable light emitting reporter domains include luciferase (from, e.g., firefly, Vargula, Renilla reniformis or Renilla muelleri), or light emitting variants thereof. Other suitable reporter domains include fluorescent proteins, (from e.g., jellyfish, corals and other coelenterates as such those from Aequoria, Renilla, Ptilosarcus, Stylatula species), or light emitting variants thereof. Light emitting variants of these reporter proteins are very well known in the art and may be brighter, dimmer, or have different excitation and/or emission spectra, as compared to a native reporter protein. For example, some variants are altered such that they no longer appear green, and may appear blue, cyan, yellow, enhanced yellow red (termed BFP, CFP, YFP eYFP and RFP, respectively) or have other emission spectra, as is known in the art. Other suitable reporter domains include domains that can report the presence of a polypeptide through a biochemical or color change, such as β-galactosidase, β-glucuronidase, chloramphenicol acetyl transferase, and secreted embryonic alkaline phosphatase.

Also as is known in the art, an affinity tags or a reporter domain may be present at any position in a polypeptide of interest. However, in most embodiments, they are present at the C- or N-terminal end of a polypeptide of interest.

2. Nucleic Acids Encoding GPCR Polypeptides of Interest

Since the genetic code and recombinant techniques for manipulating nucleic acid are known, and the amino acid sequences of GPCR polypeptides of interest described as above, the design and production of nucleic acids encoding a GPCR polypeptide of interest is well within the skill of an artisan. In certain embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) methods are used. For example, GPCR coding sequences may be isolated from a library of GPCR coding sequence using any one or a combination of a variety of recombinant methods that do not need to be described herein. Subsequent substitution, deletion, and/or addition of nucleotides in the nucleic acid sequence encoding a protein may also be done using standard recombinant DNA techniques.

For example, site directed mutagenesis and subcloning may be used to introduce/delete/substitute nucleic acid residues in a polynucleotide encoding a polypeptide of interest. In other embodiments, PCR may be used. Nucleic acids encoding a polypeptide. of interest may also be made by chemical synthesis entirely from oligonucleotides (e.g., Cello et al., Science (2002) 297:1016-8).

In some embodiments, the codons of the nucleic acids encoding polypeptides of interest are optimized for expression in cells of a particular species, particularly a mammalian, e.g., mouse, rat, hamster, non-human primate, or human, species. In some embodiments, the codons of the nucleic acids encoding polypeptides of interest are optimized for expression in cells of a particular species, particularly an amphibian species.

a. Vectors

The invention further provides vectors (also referred to as “constructs”) comprising a subject nucleic acid. In many embodiments of the invention, the subject nucleic acid sequences will be expressed in a host after the sequences have been operably linked to an expression control sequence, including, e.g. a promoter. The subject nucleic acids are also typically placed in an expression vector that can replicate in a host cell either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like. Retroviral, adenoviral and adeno-associated viral vectors may be used.

A variety of expression vectors are available to those in the art for purposes of producing a polypeptide of interest in a cell and include expression vectors which are commercially available (e.g., from Invitrogen, Carlsbad, Calif.; Clontech, Mountain View, Calif.; Stratagene, La Jolla, Calif.). Commercially available expression vectors include, by way of non-limiting example, CMV promoter-based vectors. One suitable expression vector is pCMV. The expression vector may be adenoviral. An exemplary adenoviral vector may be purchased as AdEasy™ from Qbiogene (Carlsbad, Calif.) [He T C et al, Proc Natl Acad Sci USA (1998) 95:2509-2514; and U.S. Pat. No. 5,922,576; the disclosure of each of which is herein incorporated by reference in its entirety]. Other suitable expression vectors will be readily apparent to those of ordinary skill in the art.

The subject nucleic acids usually comprise an single open reading frame encoding a subject polypeptide of interest, however, in certain embodiments, since the host cell for expression of the polypeptide of interest may be a eukaryotic cell, e.g., a mammalian cell, such as a human cell, the open reading frame may be interrupted by introns. Subject nucleic acid are typically part of a transcriptional unit which may contain, in addition to the subject nucleic acid 3′ and 5′ untranslated regions (UTRs) which may direct RNA stability, translational efficiency, etc. The subject nucleic acid may also be part of an expression cassette which contains, in addition to the subject nucleic acid a promoter, which directs the transcription and expression of a polypeptide of interest, and a transcriptional terminator.

Eukaryotic promoters can be any promoter that is functional in a eukaryotic host cell, including viral promoters and promoters derived from eukaryotic genes. Exemplary eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a promoter of the target pathogen. Promoters for use in the present invention are selected such that they are functional in the cell type (and/or animal) into which they are being introduced. In certain embodiments, the promoter is a CMV promoter.

In certain embodiments, a subject vector may also provide for expression of a selectable marker. Suitable vectors and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thymidine kinase gene, the dihydrofolate reductase gene, the xanthine-guanine phosphoribosyl transferase gene, CAD, the adenosine deaminase gene, the asparagine synthetase gene, the antibiotic resistance genes, e.g. tetr, ampr, Cmr or cat, kanr or neor (aminoglycoside phosphotransferase genes), the hygromycin B phosphotransferase gene, and the like.

As mentioned above, polypeptides of interest may be fusion proteins that contain an affinity domain and/or a reporter domain. Methods for making fusions between a reporter or tag and a GPCR, for example, at the C- or N-terminus of the GPCR, are well within the skill of one of skill in the art (e.g. McLean et al, Mol. Pharma. Mol. Pharmacol. 1999 56:1182-91; Ramsay et al., Br. J. Pharmacology, 2001, 315-323) and will not be described any further. It is expressly contemplated that such a fusion protein may contain a heterologous N-terminal domain (e.g., an epitope tag) fused in-frame with a GPCR that has had its N-terminal methionine residue either deleted or substituted with an alternative amino acid. It is appreciated that a polypeptide of interest may first be made from a native polypeptide and then operably linked to a suitable reporter/tag as described above.

The subject nucleic acids may also contain restriction sites, multiple cloning sites, primer binding sites, ligatable ends, recombination sites etc., usually in order to facilitate the construction of a nucleic acid encoding a polypeptide of interest.

b. Host Cells

The invention further provides host cells comprising a vector comprising a subject nucleic acid. Suitable host cells include prokaryotic, e.g., bacterial cells (for example E. coli), as well as eukaryotic cells e.g. an animal cell (for example an insect, mammal, fish, amphibian, bird or reptile cell), a plant cell (for example a maize or Arabidopsis cell), or a fungal cell (for example a yeast cell, a S. cerevisiae cell). In certain embodiments, any cell suitable for expression of a polypeptide of interest-encoding nucleic acid may be used as a host cell. Usually, an animal host cell line is used, examples of which are as follows: monkey kidney cells (COS cells), monkey kidney CVI cells transformed by SV40 (COS-7, ATCC CRL 165 1); human embryonic kidney cells (HEK-293 [“293”], Graham et al. J. Gen Virol. 36:59 (1977)); HEK-293T [“293T”] cells; baby hamster kidney cells (BIM, ATCC CCL 10); chinese hamster ovary-cells (CHO, Urlaub and Chasin, Proc. Natl. Acad. Sci. (USA) 77:4216, (1980); Syrian golden hamster cells MCB3901 (ATCC CRL-9595); mouse sertoli cells (TM4, Mather, Biol. Reprod. 23:243-251 (1980)); monkey kidney cells (CV1 ATCC CCL 70); african green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TR1 cells (Mather et al., Annals N.Y. Acad. Sci. 383:44-68 (1982)); NIH/3T3 cells (ATCC CRL-1658); and mouse L cells (ATCC CCL-1).

In certain embodiments, melanophores are used. Melanophores are skin cells found in lower vertebrates. Relevant materials and methods will be followed according to the disclosure of U.S. Pat. No. 5,462,856 and U.S. Pat. No. 6,051,386. These patent disclosures are herein incorporated by reference in their entirety.

In certain embodiments, yeast cells are used.

Additional cell lines will become apparent to those of ordinary skill in the art, and a wide variety of cell lines are available from the American Type Culture Collection, 10801 University Boulevard, Manassas, Va. 20110-2209.

### C. Screening of Candidate Compounds

1. Generic GPCR Screening Assay Techniques

When a G protein receptor becomes active, it binds to a G protein (e.g., Gq, Gs, Gi, Gz, Go) and stimulates the binding of GTP to the G protein. The G protein then acts as a GTPase and slowly hydrolyzes the GTP to GDP, whereby the receptor, under normal conditions, becomes deactivated. However, activated receptors continue to exchange GDP to GTP. A non-hydrolyzable analog of GTP, [³⁵S]GTPγS, can be used to monitor enhanced binding to membranes which express activated receptors. It is reported that [³⁵S]GTPγS can be used to monitor G protein coupling to membranes in the absence and presence of ligand. An example of this monitoring, among other examples well-known and available to those in the art, was reported by Traynor and Nahorski in 1995. A preferred use of this assay system is for initial screening of candidate compounds because the system is generically applicable to all G protein-coupled receptors regardless of the particular G protein that interacts with the intracellular domain of the receptor.

2. Specific GPCR Screening Assay Techniques

Once candidate compounds are identified using the “generic” G protein-coupled receptor assay (i.e., an assay to select compounds that are agonists or inverse agonists), in some embodiments further screening to confirm that the compounds have interacted at the receptor site is preferred. For example, a compound identified by the “generic” assay may not bind to the receptor, but may instead merely “uncouple” the G protein from the intracellular domain.

a. Gs, Gz and Gi.

Gs stimulates the enzyme adenylyl cyclase. Gi (and Gz and Go), on the other hand, inhibit adenylyl cyclase. Adenylyl cyclase catalyzes the conversion of ATP to cAMP; thus, activated GPCRs that couple the Gs protein are associated with increased cellular levels of cAMP. On the other hand, activated GPCRs that couple Gi (or Gz, Go) protein are associated with decreased cellular levels of cAMP. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Thus, assays that detect cAMP can be utilized to determine if a candidate compound is, e.g., an inverse agonist to the receptor (i.e., such a compound would decrease the levels of cAMP). A variety of approaches known in the art for measuring cAMP can be utilized; in some embodiments a preferred approach relies upon the use of anti-cAMP antibodies in an ELISA-based format. Another type of assay that can be utilized is a whole cell second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, an activated Gs-linked receptor causes the accumulation of cAMP that then activates the gene and expression of the reporter protein. The reporter protein such as β-galactosidase or luciferase can then be detected using standard biochemical assays (Chen et al. 1995).

b. Go and Gq.

Gq and Go are associated with activation of the enzyme phospholipase C, which in turn hydrolyzes the phospholipid PIP₂, releasing two intracellular messengers: diacyclglycerol (DAG) and inositol 1,4,5-triphosphate (IP3). Increased accumulation of IP3 is associated with activation of Gq- and Go-associated receptors. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Assays that detect IP3 accumulation can be utilized to determine if a candidate compound is, e.g., an inverse agonist to a Gq- or Go-associated receptor (i.e., such a compound would decrease the levels of IP3). Gq-associated receptors can also been examined using an AP1 reporter assay in that Gq-dependent phospholipase C causes activation of genes containing AP1 elements; thus, activated Gq-associated receptors will evidence an increase in the expression of such genes, whereby inverse agonists thereto will evidence a decrease in such expression, and agonists will evidence an increase in such expression. Commercially available assays for such detection are available.

3. GPCR Fusion Protein

The use of an endogenous, constitutively active GPCR or a non-endogenous, constitutively activated GPCR, for use in screening of candidate compounds for the direct identification of inverse agonists or agonists provides an interesting screening challenge in that, by definition, the receptor is active even in the absence of an endogenous ligand bound thereto. Thus, in order to differentiate between, e.g., the non-endogenous receptor in the presence of a candidate compound and the non-endogenous receptor in the absence of that compound, with an aim of such a differentiation to allow for an understanding as to whether such compound may be an inverse agonist or agonist or have no affect on such a receptor, in some embodiments it is preferred that an approach be utilized that can enhance such differentiation. In some embodiments, a preferred approach is the use of a GPCR Fusion Protein.

Generally, once it is determined that a non-endogenous GPCR has been constitutively activated using the assay techniques set forth above (as well as others known to the art-skilled), it is possible to determine the predominant G protein that couples with the endogenous GPCR. Coupling of the G protein to the GPCR provides a signaling pathway that can be assessed. In some embodiments it is preferred that screening take place using a mammalian or a melanophore expression system, as such a system will be expected to have endogenous G protein therein. Thus, by definition, in such a system, the non-endogenous, constitutively activated GPCR will continuously signal. In some embodiments it is preferred that this signal be enhanced such that in the presence of, e.g., an inverse agonist to the receptor, it is more likely that it will be able to more readily differentiate, particularly in the context of screening, between the receptor when it is contacted with the inverse agonist.

The GPCR Fusion Protein is intended to enhance the efficacy of G protein coupling with the GPCR. The GPCR Fusion Protein may be preferred for screening with either an endogenous, constitutively active GPCR or a non-endogenous, constitutively activated GPCR because such an approach increases the signal that is generated in such screening techniques. This is important in facilitating a significant “signal to noise” ratio; such a significant ratio is preferred for the screening of candidate compounds as disclosed herein.

The construction of a construct useful for expression of a GPCR Fusion Protein is within the purview of those having ordinary skill in the art. Commercially available expression vectors and systems offer a variety of approaches that can fit the particular needs of an investigator. Important criteria in the construction of such a GPCR Fusion Protein construct include but are not limited to, that the GPCR sequence and the G protein sequence both be in-frame (preferably, the sequence for the endogenous GPCR is upstream of the G protein sequence), and that the “stop” codon of the GPCR be deleted or replaced such that upon expression of the GPCR, the G protein can also be expressed. The GPCR can be linked directly to the G protein, or there can be spacer residues between the two (preferably, no more than about 12, although this number can be readily ascertained by one of ordinary skill in the art). Based upon convenience, it is preferred to use a spacer. In some embodiments, it is preferred that the G protein that couples to the non-endogenous GPCR will have been identified prior to the creation of the GPCR Fusion Protein construct. Because there are only a few G proteins that have been identified, it is preferred that a construct comprising the sequence of the G protein (i.e., a universal G protein construct, see Example 4(a) below) be available for insertion of a GPCR sequence therein; this provides for further efficiency in the context of large-scale screening of a variety of different GPCRs having different sequences.

As noted above, activated GPCRs that couple to Gi, Gz and Go are expected to inhibit the formation of cAMP making assays based upon these types of GPCRs challenging [i.e., the cAMP signal decreases upon activation, thus making the direct identification of, e.g., agonists (which would further decrease this signal) challenging]. As will be disclosed herein, it has been ascertained that for these types of receptors, it is possible to create a GPCR Fusion Protein that is not based upon the GPCR's endogenous G protein, in an effort to establish a viable cyclase-based assay. Thus, for example, an endogenous Gi coupled receptor can be fused to a Gs protein—such a fusion construct, upon expression, “drives” or “forces” the endogenous GPCR to couple with, e.g., Gs rather than the “natural” Gi protein, such that a cyclase-based assay can be established. Thus, for Gi, Gz and Go coupled receptors, in some embodiments it is preferred that when a GPCR Fusion Protein is used and the assay is based upon detection of adenylyl cyclase activity, that the fusion construct be established with Gs (or an equivalent G protein that stimulates the formation of the enzyme adenylyl cyclase).

TABLE C

| | Effect on | | | |
|---|---|---|---|---|
| | cAMP | | | |
| | Production | | | |
| | upon | Effect on IP3 | Effect on | |
| | Activation of | Accumulation | cAMP | Effect on IP3 |
| | GPCR (i.e., | upon Activation | Production | Accumulation |
| | constitutive | of GPCR (i.e., | upon contact | upon contact |
| | activation or | constitutive | with an | with |
| G | agonist | activation or | Inverse | an Inverse |
| protein | binding) | agonist binding) | Agonist | Agonist |
| Gs | Increase | N/A | Decrease | N/A |
| Gi | Decrease | N/A | Increase | N/A |
| Gz | Decrease | N/A | Increase | N/A |
| Go | Decrease | Increase | Increase | Decrease |
| Gq | N/A | Increase | N/A | Decrease |

Equally effective is a G Protein Fusion construct that utilizes a Gq Protein fused with a Gs, Gi, Gz or Go Protein. In some embodiments a preferred fusion construct can be accomplished with a Gq Protein wherein the first six (6) amino acids of the G-protein a-subunit (“Gaq”) is deleted and the last five (5) amino acids at the C-terminal end of Gαq is replaced with the corresponding amino acids of the Gα of the G protein of interest. For example, a fusion construct can have a Gq (6 amino acid deletion) fused with a G1Protein, resulting in a “Gq/Gi Fusion Construct”. This fusion construct will force the endogenous Gi coupled receptor to couple to its non-endogenous G protein, Gq, such that the second messenger, for example, inositol triphosphate or diacylgycerol, can be measured in lieu of cAMP production.

4. Co-transfection of a Target Gi Coupled GPCR with a Signal-Enhancer Gs Coupled GPCR (cAMP Based Assays)

A Gi coupled receptor is known to inhibit adenylyl cyclase, and, therefore, decreases the level of cAMP production, which can make the assessment of cAMP levels challenging. In certain embodiments, an effective technique in measuring the decrease in production of cAMP as an indication of activation of a receptor that predominantly couples Gi upon activation can be accomplished by co-transfecting a signal enhancer, e.g., a non-endogenous, constitutively activated receptor that predominantly couples with Gs upon activation (e.g., TSHR-A6231; see infra), with the Gi linked GPCR. As is apparent, activation of a Gs coupled receptor can be determined based upon an increase in production of cAMP. Activation of a Gi coupled receptor leads to a decrease in production cAMP. Thus, the co-transfection approach is intended to advantageously exploit these “opposite” affects. For example, co-transfection of a non-endogenous, constitutively activated Gs coupled receptor (the “signal enhancer”) with expression vector alone provides a baseline cAMP signal (i.e., although the Gi coupled receptor will decrease cAMP levels, this “decrease” will be relative to the substantial increase in cAMP levels established by constitutively activated Gs coupled signal enhancer). By then co-transfecting the signal enhancer with the “target receptor”, an inverse agonist of the Gi coupled target receptor will increase the measured cAMP signal, while an agonist of the Gi coupled target receptor will decrease this signal.

Candidate compounds that are directly identified using this approach should be assessed independently to ensure that these do not target the signal enhancing receptor (this can be done prior to or after screening against the co-transfected receptors).

### D. Exemplary BRS-3 Agonists

Exemplary BRS-3 agonists useful in methods of the present invention include the compounds provided in Table D. The compounds in Table D additionally are exemplary ligands of BRS-3.

Examples of BRS-3 agonists are described in Weber et al., J Med Chem (2003) 46:1918-1930, the disclosure of which is herein incorporated by reference in its entirety.

Examples of BRS-3 agonists are described in Mantey et al., J Pharmacol Exp Ther (2004) 310:1161-1169, the disclosure of which is herein incorporated by reference in its entirety.

Examples of BRS-3 agonists are described in Boyle et al., J Peptide Sci (2005) 11:136-141, the disclosure of which is herein incorporated by reference in its entirety.

Examples of BRS-3 agonists are described in Lamerich et al., Br J Pharmacol (2003) 138:1431-1440, the disclosure of which is herein incorporated by reference in its entirety.

Examples of BRS-3 agonists are described in Gonzalez et al, J Pharmacol Exp Ther (2007 Nov. 15), the disclosure of which is herein incorporated by reference in its entirety.

In certain embodiments, the BRS-3 agonist is a compound selected from Table D.

In certain embodiments, the BRS-3 agonist is a compound selected from Compound D1, Compound D2, Compound D3, Compound D4, Compound D5, Compound D6, Compound D7, Compound D8, Compound D9, Compound D10, Compound D11, Compound D12, Compound D13, Compound D14, Compound D15, Compound D16, Compound D17, Compound D18, Compound D19, Compound D₂₀, Compound D21, Compound D22, Compound D23, Compound D24, Compound D25, Compound D26, and Compound D27; these Compounds may be found in Weber et al., J Med Chem (2003) 46:1918-1930, where they are described as selective BRS-3 agonists.

In certain embodiments, the BRS-3 agonist is a compound selected from Compound D28, Compound D29, and Compound D30; these Compounds may be found in Mantey et al., J Pharmacol Exp Ther (2004) 310:1161-1169, where they are described as selective BRS-3 agonists.

In certain embodiments, the BRS-3 agonist is a compound selected from Compound D31, Compound D32, and Compound D33; these Compounds may be found in Boyle et al., J Peptide Sci (2005) 11:136-141, where they are described as selective BRS-3 agonists.

In certain embodiments, the BRS-3 agonist is Compound D34; this Compound may be found in Mantey et al., J Biol Chem (1997) 272:26062-26071, where it is described as a BRS-3 agonist non-selective for BRS-3 over GRP-R and NMB-R.

In certain embodiments, the BRS-3 agonist is Compound D35; this Compound may be found in Mantey et al., J Biol Chem (1997) 272:26062-26071, where it is described as a BRS-3 agonist.

TABLE D

| Compound No. | Compound |
|---|---|
| D1 | N1-{(1R)-2-(1H-3-Indolyl)-1-[1-(2-phenylethyl)carbamoyl]ethyl}-(2S)-2-[1- |
| | (2-(4-chlorophenyl)ethyl)carboxamido]pentanediamide |
| D2 | N1-{(1R)-2-(1H-3-Indolyl)-1-[1-(2-phenylethyl)carbamoyl]ethyl}-(2S)-2- |
| | [(1H-2-indolylmethyl)carboxamido]pentanediamide |
| D3 | N1-(2-Phenylethyl)-(2R)-2-{[(1S)-1- |
| | (benzylcarboxamido)ethyl]carboxamido}-3-(1H-3-indolyl)propanamide |
| D4 | N1-(2-Phenylethyl)-(2R)-2-{[(1S)-1-((4-chlorobenzyl)- |
| | carboxamido)ethyl]carboxamido}-3-(1H-3-indolyl)propanamide |
| D5 | N1-(2-Phenylethyl)-(2R)-2-{[(1S)-1-((1,3-benzodioxol-5- |
| | ylmethyl)carboxamido)ethyl]carboxamido}-3-(1H-3-indolyl)propanamide |
| D6 | N1-(2-Phenylethyl)-(2R)-2-{[(1S)-1-((3- |
| | pyridyl)methylcarboxamido)ethyl]carboxamido}-3-(1H-3- |
| | indolyl)propanamide |
| D7 | N1-(2-Phenylethyl)-(2R)-2-{[(1S)-1-((1,2,3,4-tetrahydro-1- |
| | isoquinolinyl)methylcarboxamido)ethyl]carboxamido}-3-(1H-3- |
| | indolyl)propanamide |
| D8 | N1-(2-Phenylethyl)-(2R)-2-{[(1S)-1-((1H2- |
| | indolyl)methylcarboxamido)ethyl]carboxamido}-3-(1H-3- |
| | indolyl)propanamide |
| D9 | N1-(2-Phenylethyl)-(2R)-2-{[(benzyl)amino]methylcarboxamido}-3-(1H-3- |
| | indolyl)propanamide |
| D10 | N1-(2-Phenylethyl)-(2R)-2-{[(4-chlorobenzyl)amino]-methylcarboxamido}- |
| | 3-(1H-3-indolyl)propanamide |
| D11 | N1-(2-Phenylethyl)-(2R)-2-{[(3-pyridyl)methylamino]- |
| | methylcarboxamido}-3-(1H-3-indolyl)propanamide |
| D12 | N1-(2-Phenylethyl)-(2R)-2-{[1-(2-phenylethyl)amino]-methylcarboxamido}- |
| | 3-(1H-3-indolyl)propanamide |
| D13 | N1-(2-Phenylethyl)-(2R)-2-{[1-benzylamino)ethyl]carboxamido}-3-(1H-3- |
| | indolyl)propanamide |
| D14 | N1-(2-Phenylethyl)-(2R)-2-{[1-((4- |
| | chlorobenzyl)amino)ethyl]carboxamido}-3-(1H-3-indolyl)propanamide |
| D15 | N1-(2-Phenylethyl)-(2R)-2-{[1-(1-(2- |
| | phenylethyl)amino)ethyl]carboxamido}-3-(1H-3-indolyl)propanamide |
| D16 | N1-(2-Phenylethyl)-(2R)-2-{{N′-(benzoyl)hydrazino]carboxamido}-3-(1H- |
| | 3-indolyl)propanamide |
| D17 | N1-(2-Phenylethyl)-(2R)-2-{{N′-(4-chlorobenzoyl)- |
| | hydrazino]carboxamido}-3-(1H-3-indolyl)propanamide |
| D18 | N1-(2-Phenylethyl)-(2R)-2-{[N′-(2-(3- |
| | pyridyl)ethanoyl)hydrazino]carboxamido}-3-(1H-3-indolyl)propanamide |
| D19 | N1-(2-Phenylethyl)-(2R)-2-{{N′-(2-(1,2,3,4-tetrahydro-1- |
| | isoquinolinyl)ethanoyl)hydrazino]carboxamido}-3-(1H-3- |
| | indolyl)propanamide |
| D20 | N1-(2-Phenylethyl)-(2R)-2-{[N′-(2-(1H-2- |
| | indolyl)ethanoyl)hydrazino]carboxamido}-3-(1H-3-indolyl)propanamide |
| D21 | N1-(2-Phenylethyl)-(2R)-2-{[N′-(phenylmethylene)- |
| | hydrazino]carboxamido}-3-(1H-3-indolyl)propanamide |
| D22 | N1-(2-Phenylethyl)-(2R)-2-{[N′-(furan-2- |
| | ylmethylene)hydrazino]carboxamido}-3-(1H-3-indolyl)propanamide |
| D23 | N1-(2-Phenylethyl)-(2R)-2-{[N′-(benzyl)hydrazino]carboxamido}-3-(1H-3- |
| | indolyl)propanamide |
| D24 | N1-(2-Phenylethyl)-(2R)-2-{[N′-(4-chlorobenzyl)hydrazino]carboxamido}- |
| | 3-(1H-3-indolyl)propanamide |
| D25 | N1-(2-Phenylethyl)-(2R)-2-{[N′-(2-furylmethyl)hydrazino]carboxamido}-3- |
| | (1H-3-indolyl)propanamide |
| D26 | N1-(2-Phenylethyl)-(2R)-2-{[4-benzylpiperazino)methyl]carboxamido}-3- |
| | (1H-3-indolyl)propanamide |
| D27 | N1-(2-Phenylethyl)-(2R)-2-{[(4-benzylpiperidino)methyl]carboxamido}-3- |
| | (1H-3-indolyl)propanamide |
| D28 | [D-Tyr⁶,(R)-Apa¹¹,Phe¹³,Nle¹⁴]Bombesin(6-14) |
| | (where Apa represents aminopropionic acid) |
| D29 | [D-Tyr⁶,(S)-Apa¹¹,Phe¹³,Nle¹⁴]Bombesin(6-14) |
| | (where Apa represents aminopropionic acid) |
| D30 | [D-Tyr⁶,(R)-Apa¹¹-4Cl,Phe¹³,Nle¹⁴]Bombesin(6-14) |
| | (where Apa represents aminopropionic acid) |
| D31 | Ac-Phe-Trp-Ala-His(τBzl)-Nip-Gly-Arg-NH₂ |
| | (where Nip represents piperidine-3-carboxylic acid) |
| D32 | Ac-Phe⁷-Trp⁸-Ala⁹-His(τBzl)¹⁰-βAla¹¹-His¹²-Arg¹³-NH₂ |
| D33 | Ac-Phe⁷-Trp⁸-Ala⁹-Val¹⁰-βAla¹¹-His¹²-Arg¹³-Trp-NH₂ |
| D34 | [D-Tyr⁶,βAla¹¹,Phe¹³,Nle¹⁴]Bombesin(6-14) |
| | (where Nle represents norleucine) |
| D35 | [D-Phe⁶,β-Ala¹¹,Phe¹³,Nle¹⁴]Bombesin(6-14) |
| | (where Nle represents norleucine) |

It is expressly contemplated that the compounds in Table D can be used individually or in any combination in any embodiment of the present invention. It is also expressly contemplated that the compounds in Table D can be excluded individually or in any combination from any embodiment of the present invention.

Additionally, compounds of the invention, including those listed in Table D, encompass also pharmaceutically acceptable salts, hydrates, solvates, geometrical isomers, tautomers, and optical isomers thereof. See, e.g., Berge et al (1977), Journal of Pharmaceutical Sciences 66:1-19; and Polymorphism in Pharmaceutical Solids (1999) Brittain, ed., Marcel Dekker, Inc.; the disclosure of each of which is herein incorporated by reference in its entirely.

### E. Exemplary BRS-3 Antagonists

Exemplary BRS-3 antagonists useful in methods of the present invention include the compounds provided in Table E. The compounds in Table E additionally are exemplary ligands of BRS-3.

Examples of BRS-3 antagonists are described in Ryan et al., J Biol Chem (1998) 273:13613-13624, the disclosure of which is herein incorporated by reference in its entirety.

Examples of BRS-3 antagonists are described in International Application No. PCT/GB2004/005169 (published as WO 2005/056532), the disclosure of which is herein incorporated in its entirety. Disclosed in International Application No. PCT/GB2004/005169 as a BRS-3 antagonist is a compound of Formula (I):

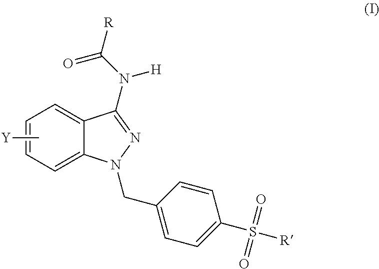

wherein:

R is aryl-C₁₋₆-alkyl, heteroaryl-C₁₋₆-alkyl, aryloxy-C₁₋₆-alkyl or heteroaryloxy-C₁₋₆-alkyl; and R is optionally independently substituted with one or more of C₁₋₆-alkoxy, C₁₋₆-alkyl, methylenedioxy, aryl, halogen and halo C₁₋₆-alkyl;

R′ is C₁₋₆-dialkyl amine, C₁₋₆-alkyl amine, C₄₋₇-cyclic alkyl amine or C₃₋₈-cycloalkyl amine; and R′ is optionally independently substituted with one or more of C₁₋₆-alkoxy; and

Y is hydrogen, C₁₋₆-alkyl, C₁₋₆-alkoxy or halogen.

Specific examples of BRS-3 antagonists disclosed in International Application No. PCT/GB2004/005169 include the following compounds according to Formula (I) (referred to herein as Group A1): N-[1-(4-Diethylsulfamoyl-benzyl)-1H-indazol-3-yl]-2-phenoxy-acetamide; 2-Benzo[1,3] dioxo-5-yl-N-[1-(4-diethylsulfamoyl-benzyl)-1H-indazol-3-yl]-acetamide; 2,3-Dihydro-benzo[1,4] dioxine-2-carboxylic acid [1-(4-diethylsulfamoyl-benzyl)-1H-indazol-3-yl]-amide; N-[1-(4-Diethylsulfamoyl-benzyl)-1H-indazol-3-yl]-2-(3-methoxy-phenyl)-acetamide; 2-Phenoxy-N-{1-[4-(pyrrolidine-1-sulfonyl)-benzyl]-1H-indazol-3-yl} propionamide; 2-(4-Chloro-phenoxy-N-[1-(4-diethylsulfamoyl-benzyl)-1H-indazol-3-yl]-acetamide; N-[1-(4-Diethylsulfamoyl-benzyl)-1H-indazol-3-yl]-2-(3,4-dimethoxy-phenyl)-acetamide; N-[1-(4-Dipropylsulfamoyl-benzyl)-1H-indazol-3-yl]-2-phenoxy-acetamide; N-[1-(4-Dipropylsulfamoyl-benzyl)-1H-indazol-3-yl]-2-phenoxy-propionamide; 2,3-Dihydro-benzo[1,4] dioxine-2-carboxylic acid [1-(4-dipropylsulfamoyl-benzyl)1H-indazol-3-yl]-amide; 2-(4-Chloro-phenoxy)-N-[1-(4-dipropylsulfamoyl-benzyl)-1H-indazol-3-yl]-acetamide; 2-(3,4-Dimethoxy-phenyl)-N-[1-(4-dipropylsulfamoyl-benzyl)-1H-indazol-3-yl]-acetamide; N-[1-(4-Dipropylsulfamoyl-benzyl)-1H-indazol-3-yl]-2-(4-methoxy-phenyl)-acetamide; N-[1-(4-Dipropylsulfamoyl-benzyl)-1H-indazol-3-yl]-2-(3-methoxy-phenyl)-acetamide; 2,3-Dihydro-benzofuran-2-carboxylic acid [1-(4-dipropylsulfamoyl-benzyl)-1H-indazol-3-yl]-amide; 2,3-Dihydro-benzo[1,4] dioxine-2-carboxylic acid [1-(4-ethylsulfamoyl-benzyl)-1H-indazol-3-yl]-amide; 2,3-Dihydro-benzo[1,4] dioxine-2-carboxylic acid [1-(4-dimethylsulfamoyl-benzyl)-1H-indazol-3-yl]-amide; 2-(3,4-Dimethoxy-phenyl)-N-[1-(4-dimethylsulfamoyl-benzyl)-1H-indazol-3-yl]-acetamide; N-[1-4-Diethylsulfamoyl-benzyl)-1H-indazol-3-yl]-2-phenoxy-propionamide; and 2,3-Dihydro-benzofuran-2-carboxylic acid [1-(4-diethylsulfamoyl-benzyl)-1H-indazol-3-yl]-amide.

In certain embodiments, the BRS-3 antagonist is a compound selected from Table E.

In certain embodiments, the BRS-3 antagonist is a compound selected from Compound E1 and Compound E2; these Compounds may be found in Ryan et al., J Biol Chem (1998) 273:13613-13624, where they are described as BRS-3 antagonists.

In certain embodiments, the BRS-3 antagonist is a compound selected from Compound E3, Compound E4, Compound E5, Compound E6, Compound E7, Compound E8, Compound E9, Compound E10, Compound E11, Compound E12, Compound E13, Compound E14, Compound E15, Compound E16, Compound E17, Compound E18, Compound E19, Compound E20, Compound E21, and Compound E22; these Compounds may be found in International Application No. PCT/GB2004/005169 (published as WO 2005/056532), where they are disclosed as BRS-3 antagonists.

TABLE E

| Compound No. | Compound |
|---|---|
| E1 | D-Nal,Cys,Tyr,D-Trp,Lys,Val,Cys,Nal-NH₂ |
| | (where Nal represents β-naphthylalanine) |
| E2 | [D-Pro⁴,D-Trp^{7,9,10}]SP(4-11) |
| | (where SP represents Substance P) |
| E3 | N-[1-(4-Diethylsulfamoyl-benzyl)-1H-indazol-3-yl]-2-phenoxy-acetamide |
| E4 | 2-Benzo[1,3]dioxo-5-yl-N-[1-(4-diethylsulfamoyl-benzyl)-1H-indazol-3-yl]- |
| | acetamide |
| E5 | 2,3-Dihydro-benzo[1,4]dioxine-2-carboxylic acid [1-(4-diethylsulfamoyl- |
| | benzyl)-1H-indazol-3-yl]-amide |
| E6 | N-[1-(4-Diethylsulfamoyl-benzyl)-1H-indazol-3-yl]-2-(3-methoxy-phenyl)- |
| | acetamide |
| E7 | 2-Phenoxy-N-{1-[4-(pyrrolidine-1-sulfonyl)-benzyl]-1H-indazol-3- |
| | yl}propionamide |
| E8 | 2-(4-Chloro-phenoxy-N-[1-(4-diethylsulfamoyl-benzyl)-1H-indazol-3-yl]- |
| | acetamide |
| E9 | N-[1-(4-Diethylsulfamoyl-benzyl)-1H-indazol-3-yl]-2-(3,4-dimethoxy- |
| | phenyl)-acetamide |
| E10 | N-[1-(4-Dipropylsulfamoyl-benzyl)-1H-indazol-3-yl]-2-phenoxy-acetamide |
| E11 | N-[1-(4-Dipropylsulfamoyl-benzyl)-1H-indazol-3-yl]-2-phenoxy- |
| | propionamide |
| E12 | 2,3-Dihydro-benzo[1,4]dioxine-2-carboxylic acid [1-(4-dipropylsulfamoyl- |
| | benzyl)1H-indazol-3-yl]-amide |
| E13 | 2-(4-Chloro-phenoxy)-N-[1-(4-dipropylsulfamoyl-benzyl)-1H-indazol-3-yl]- |
| | acetamide |
| E14 | 2-(3,4-Dimethoxy-phenyl)-N-[1-(4-dipropylsulfamoyl-benzyl)-1H-indazol- |
| | 3-yl]-acetamide |
| E15 | N-[1-(4-Dipropylsulfamoyl-benzyl)-1H-indazol-3-yl]-2-(4-methoxy- |
| | phenyl)-acetamide |
| E16 | N-[1-(4-Dipropylsulfamoyl-benzyl)-1H-indazol-3-yl]-2-(3-methoxy- |
| | phenyl)-acetamide |
| E17 | 2,3-Dihydro-benzofuran-2-carboxylic acid [1-(4-dipropylsulfamoyl-benzyl)- |
| | 1H-indazol-3-yl]-amide |
| E18 | 2,3-Dihydro-benzo[1,4]dioxine-2-carboxylic acid [1-(4-ethylsulfamoyl- |
| | benzyl)-1H-indazol-3-yl]-amide |
| E19 | 2,3-Dihydro-benzo[1,4]dioxine-2-carboxylic acid [1-(4-dimethylsulfamoyl- |
| | benzyl)-1H-indazol-3-yl]-amide |
| E20 | 2-(3,4-Dimethoxy-phenyl)-N-[1-(4-dimethylsulfamoyl-benzyl)-1H-indazol- |
| | 3-yl]-acetamide |
| E21 | N-[1-4-Diethylsulfamoyl-benzyl)-1H-indazol-3-yl]-2-phenoxy- |
| | propionamide |
| E22 | 2,3-Dihydro-benzofuran-2-carboxylic acid [1-(4-diethylsulfamoyl-benzyl)- |
| | 1H-indazol-3-yl]-amide |

It is expressly contemplated that the compounds in Table E can be used individually or in any combination in any embodiment of the present invention. It is also expressly contemplated that the compounds in Table E can be excluded individually or in any combination from any embodiment of the present invention.

Additionally, compounds of the invention, including those listed in Table E, encompass also pharmaceutically acceptable salts, hydrates, solvates, geometrical isomers, tautomers, and optical isomers thereof. See, e.g., Berge et al (1977), Journal of Pharmaceutical Sciences 66:1-19; and Polymorphism in Pharmaceutical Solids (1999) Brittain, ed., Marcel Dekker, Inc.; the disclosure of each of which is herein incorporated by reference in its entirely.

### F. Medicinal Chemistry

Candidate Compounds

Any molecule known in the art can be tested for its ability to modulate (increase or decrease) the activity of a GPCR of the present invention. For identifying a compound that modulates activity, candidate compounds can be directly provided to a cell expressing the receptor.

This embodiment of the invention is well suited to screen chemical libraries for molecules which modulate, e.g., inhibit, antagonize, or agonize, the amount of, or activity of, a receptor. The chemical libraries can be peptide libraries, peptidomimetic libraries, chemically synthesized libraries, recombinant, e.g., phage display libraries, and in vitro translation-based libraries, other non-peptide synthetic organic libraries, etc. This embodiment of the invention is also well suited to screen endogenous candidate compounds comprising biological materials, including but not limited to plasma and tissue extracts, and to screen libraries of endogenous compounds known to have biological activity.

In some embodiments, direct identification of candidate compounds is conducted in conjunction with compounds generated via combinatorial chemistry techniques, whereby thousands of compounds are randomly prepared for such analysis. The candidate compound may be a member of a chemical library. This may comprise any convenient number of subject members, for example tens to hundreds to thousand to millions of suitable compounds, for example peptides, peptoids and other oligomeric compounds (cyclic or linear), and template-based smaller molecules, for example benzodiazepines, hydantoins, biaryls, carbocyclic and polycyclic compounds (e.g., naphthalenes, phenothiazines, acridines, steroids etc.), carbohydrate and amino acid derivatives, dihydropyridines, benzhydryls and heterocycles (e.g., trizines, indoles, thiazolidines etc.). The numbers quoted and the types of compounds listed are illustrative, but not limiting. Preferred chemical libraries comprise chemical compounds of low molecular weight and potential therapeutic agents.

Exemplary chemical libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

In another embodiment of the present invention, combinatorial chemistry can be used to identify modulators of the GPCRs of the present invention. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the subject library members for other proteins or receptors of interest (in the instant invention, the receptors of the present invention). The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J.H. Skerritt. Editors, 1995, AOAC: Washington, D.C., 305-312).

In some embodiments, the candidate compound is a polypeptide. In some embodiments, the candidate compound is not a polypeptide. In some embodiments, the candidate compound is not a peptoid. In some preferred embodiments, the candidate compound is a small molecule. In some embodiments, the candidate compound is not an antibody or an antigen-binding fragment thereof.

Candidate Compounds Identified as Modulators

Generally, the results of such screening will be compounds having unique core structures; thereafter, these compounds may be subjected to additional chemical modification around a preferred core structure(s) to further enhance the medicinal properties thereof. Such techniques are known to those in the art and will not be addressed in detail in this patent document.

In certain embodiments, a modulator of the invention is orally active. A number of computational approaches available to those of ordinary skill in the art have been developed for prediction of oral bioavailability of a drug [Ooms et al., Biochim Biophys Acta (2002) 1587:118-25; Clark & Grootenhuis, Curr OpinDrug Discov Devel (2002) 5:382-90; Cheng et al., J Comput Chem (2002) 23:172-83; Norinder & Haeberlein, Adv Drug Deliv Rev (2002) 54:291-313; Matter et al., Comb Chem High Throughput Screen (2001) 4:453-75; Podlogar & Muegge, Curr Top Med Chem (2001) 1:257-75; the disclosure of each of which is herein incorporated. by reference in its entirety). Furthermore, positron emission tomography (PET) has been successfully used by a number of groups to obtain direct measurements of drug distribution, including an assessment of oral bioavailability, in the mammalian body following oral administration of the drug, including non-human primate and human body [Noda et al., J Nucl Med (2003) 44:105-8; Gulyas et al., Eur J Nucl Med Mol Imaging (2002) 29:1031-8; Kanerva et al., Psychopharmacology (1999) 145:76-81; the disclosure of each of which is herein incorporated by reference in its entirety]. In some embodiments, a modulator of the invention is orally active.

In certain embodiments, a modulator of the invention which is orally active is able to cross the blood-brain barrier. A number of computational approaches available to those of ordinary skill in the art have been developed for prediction of the permeation of the blood-brain barrier [Ooms et al., Biochim Biophys Acta (2002) 1587:118-25; Clark & Grootenhuis, Curr OpinDrug Discov Devel (2002) 5:382-90; Cheng et al., J Comput Chem (2002) 23:172-83; Norinder & Haeberlein, Adv Drug Deliv Rev (2002) 54:291-313; Matter et al., Comb Chem High Throughput Screen (2001) 4:453-75; Podlogar & Muegge, Curr Top Med Chem (2001) 1:257-75; the disclosure of each of which is herein incorporated by reference in its entirety). A number of in vitro methods have been developed to predict blood-brain barrier permeability of drugs [Lohmann et al., J Drug Target (2002) 10:263-76; Hansen et al., J Pharm Biomed Anal (2002) 27:945-58; Otis et al., J Pharmocol Toxicol Methods (2001) 45:71-7; Dehouck et al, J Neurochem (1990) 54:1798-801; the disclosure of each of which is herein incorporated by reference in its entirety]. Furthermore, a number of strategies have been developed to enhance drug delivery across the blood-brain barrier [Scherrmann, Vascul Pharmacol (2002) 38:349-54; Pardridge, Arch Neurol (2002) 59:35-40; Pardridge, Neuron (2002) 36:555-8; the disclosure of each of which is hereby incorporated by reference in its entirety]. Finally, positron emission tomography (PET) has been successfully used by a number of groups to obtain direct measurements of drug distribution, including that within brain, in the mammalian body, including non-human primate and human body [Noda et al., J Nucl Med (2003) 44:105-8; Gulyas et al., Eur J Nucl Med Mol Imaging (2002) 29:1031-8; Kanerva et al., Psychopharmacology (1999) 145:76-81; the disclosure of each of which is herein incorporated by reference in its entirety].

In some embodiments, said modulator is selective for BRS-3 receptor, wherein a modulator selective for BRS-3 receptor is understood to refer to a modulator having selectivity for BRS-3 over one or more closely related receptors, such as gastric-releasing peptide receptor (GRP-R; e.g., human GRP-R, GenBank® Accession No. NP 005305) or neuromedin B receptor (NMB-R; e.g., human NMB-R, GenBank® Accession No. NP_{—}002502). In certain embodiments, a BRS-3 selective modulator is a BRS-3 selective inverse agonist or antagonist having a selectivity for BRS-3 over GRP-R or NMB-R of at least about 10-fold or of at least about 100-fold. In certain embodiments, a BRS-3 selective modulator is a BRS-3 selective inverse agonist or antagonist having a selectivity for BRS-3 over GRP-R and NMB-R of at least about 10-fold or of at least about 100-fold. In certain embodiments, a BRS-3 selective modulator is a BRS-3 selective agonist or partial agonist having a selectivity for BRS-3 over GRP-R or NMB-R of at least about 10-fold or of at least about 100-fold. In certain embodiments, a BRS-3 selective modulator is a BRS-3 selective agonist or partial agonist having a selectivity for BRS-3 over GRP-R and NMB-R of at least about 10-fold or of at least about 100-fold. In some preferred embodiments, BRS-3 is human BRS-3.

In some embodiments, the modulator is an inverse agonist or antagonist with an IC₅₀ of less than about 10 μM, of less than about 1 μM, of less than about 100 nM, or of less than about 10 nM at human BRS-3. In some embodiments, the modulator is an inverse agonist or antagonist with an IC₅₀ of less than a value selected from the interval of about 10 nM to 10 μM. In some embodiments, modulator is an inverse agonist or antagonist with an IC₅₀ of less than a value selected from the interval of about 10 nM to 1 μM. In some embodiments, the modulator is an inverse agonist or antagonist with an IC₅₀ of less than a value selected from the interval of about 10 nM to 100 nM. In some embodiments, the modulator is an inverse agonist or antagonist with an IC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the modulator is an inverse agonist or antagonist with an IC₅₀ of less than about 10 μM, of less than about 1 μM, of less than about 100 nM, or of less than about 10 nM in said assay. In some embodiments, said modulator is an inverse agonist or antagonist with an IC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the modulator is an inverse agonist or antagonist with an IC₅₀ in said assay of less than a value selected from the interval of about 10 nM to 10 μM. In some embodiments, the modulator is an inverse agonist or antagonist with an IC₅₀ in said assay of less than a value selected from the interval of about 10 nM to 1 μM. In some embodiments, the modulator is an inverse agonist or antagonist with an IC₅₀ in said assay of less than a value selected from the interval of about 10 nM to 100 nM.

In one aspect of the present invention, the BRS-3 inverse agonist or antagonist is a selective BRS-3 inverse agonist or antagonist, wherein the selective BRS-3 inverse agonist or antagonist has a selectivity for BRS-3 over gastric-releasing peptide receptor (GRP-R; e.g., human GRP-R, GenBank® Accession No. NP_{—}005305) or neuromedin B receptor (NMB-R; e.g., human NMB-R, GenBank® Accession No. NP_{—}002502) of at least about 10-fold, more preferably of at least about 100-fold. In one aspect of the present invention, the BRS-3 inverse agonist or antagonist is a selective BRS-3 inverse agonist or antagonist, wherein the selective BRS-3 inverse agonist or antagonist has a selectivity for BRS-3 over gastric-releasing peptide receptor (GRP-R) and neuromedin B receptor (NMB-R) of at least about 10-fold, more preferably of at least about 100-fold.

In some embodiments, the modulator is an agonist or partial agonist with an EC₅₀ of less than about 10 μM, of less than about 1 μM, of less than about 100 nM, or of less than about 10 nM at human BRS-3. In some embodiments, the modulator is an agonist or partial agonist with an EC₅₀ of less than a value selected from the interval of about 10 nM to 10 μM. In some embodiments, modulator is an agonist or partial agonist with an EC₅₀ of less than a value selected from the interval of about 10 nM to 1 μM. In some embodiments, the modulator is an agonist or partial agonist with an EC₅₀ of less than a value selected from the interval of about 10 nM to 100 nM. In some embodiments, the modulator is an agonist or partial agonist with an EC₅₀ of less than about 10 μM, of less than about 1 μM, of less than about 100 nM, or of less than about 10 nM in GTPγS binding assay carried out with membrane from transfected CHO cells, or in pigment dispersion assay carried out in transfected melanophores, or in FLIPR assay carried out in transfected HeLa cells, or in IP3 assay carried out in transfected COS-7 cells or CHO cells or HeLa cells, wherein the transfected CHO cells or the transfected melanophore cells or the transfected COS-7 cells or the transfected HeLa cells express a recombinant BRS-3 receptor having the amino acid sequence of SEQ ID NO: 2. In some embodiments, the modulator is an agonist or partial agonist with an EC₅₀ of less than about 10 μM, of less than about 1 μM, of less than about 100 nM, or of less than about 10 nM in said assay. In some embodiments, said modulator is an agonist or partial agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, the modulator is an agonist or partial agonist with an EC₅₀ in said assay of less than a value selected from the interval of about 10 nM to 10 μM. In some embodiments, the modulator is an agonist or partial agonist with an EC₅₀ in said assay of less than a value selected from the interval of about 10 nM to 1 μM. In some embodiments, the modulator is an agonist or partial agonist with an EC₅₀ in said assay of less than a value selected from the interval of about 10 nM to 100 nM.

In one aspect of the present invention, the BRS-3 agonist or partial agonist is a selective BRS-3 agonist or partial agonist, wherein the selective BRS-3 agonist or partial agonist has a selectivity for BRS-3 over gastric-releasing peptide receptor (GRP-R) or neuromedin B receptor (NMB-R) of at least about 10-fold, more preferably of at least about 100-fold. In one aspect of the present invention, the BRS-3 agonist or partial agonist is a selective BRS-3 agonist or partial agonist, wherein the selective BRS-3 agonist or partial agonist has a selectivity for BRS-3 over gastric-releasing peptide receptor (GRP-R) and neuromedin B receptor (NMB-R) of at least about 10-fold, more preferably of at least about 100-fold.

### G. Indications and Methods of Treatment

Sleep Architecture

Sleep architecture refers to organization of sleep into several stages and the distribution of those stages across time. Sleep comprises two physiological states: Non rapid eye movement (NREM) and rapid eye movement (REM) sleep. NREM sleep consists of four stages, each of which is characterized by progressively slower brain wave patterns, with the slower patterns indicating deeper sleep. So called delta sleep, stages 3 and 4 of NREM sleep, is the deepest and most refreshing type of sleep. Many patients with sleep disorders are unable to adequately achieve the restorative sleep of stages 3 and 4. In clinical terms, patients' sleep patterns are described as fragmented, meaning the patient spends a lot of time alternating between stages 1 and 2 (semi-wakefulness) and being awake and very little time in deep sleep. As used herein, the term “fragmented sleep architecture” means an individual, such as a sleep disorder patient, spends the majority of their sleep time in NREM sleep stages 1 and 2, lighter periods of sleep from which the individual can be easily aroused to a Waking state by limited external stimuli. As a result, the individual cycles through frequent bouts of light sleep interrupted by frequent awakenings throughout the sleep period. Many sleep disorders are characterized by a fragmented sleep architecture. For example, many elderly patients with sleep complaints have difficulty achieving long bouts of deep refreshing sleep (NREM stages 3 and 4) and instead spend the majority of their sleep time in NREM sleep stages 1 and 2.

In contrast to fragmented sleep architecture, as used herein the term “sleep consolidation” means a state in which the number of NREM sleep bouts, particularly Stages 3 and 4, and the length of those sleep bouts are increased, while the number and length of waking bouts are decreased. In essence, the architecture of the sleep disorder patient is consolidated to a sleeping state with increased periods of sleep and fewer awakenings during the night and more time is spent in slow wave sleep (Stages 3 and 4) with fewer oscillation Stage 1 and 2 sleep. Compounds of the present invention can be effective in consolidating sleep patterns so that the patient with previously fragmented sleep can now achieve restorative, delta-wave sleep for longer, more consistent periods of time.

As sleep moves from stage 1 into later stages, heart rate and blood pressure drop, metabolic rate and glucose consumption fall, and muscles relax. In normal sleep architecture, NREM sleep makes up about 75% of total sleep time; stage 1 accounting for 5-10% of total sleep time, stage 2 for about 45-50%, stage 3 approximately 12%, and stage 4 13-15%. About 90 minutes after sleep onset, NREM sleep gives way to the first REM sleep episode of the night. REM makes up approximately 25% of total sleep time. In contrast to NREM sleep, REM sleep is characterized by high pulse, respiration, and blood pressure, as well as other physiological patterns similar to those seen in the active waking stage. Hence, REM sleep is also known as “paradoxical sleep.” Sleep onset occurs during NREM sleep and takes 10-20 minutes in healthy young adults. The four stages of NREM sleep together with a REM phase form one complete sleep cycle that is repeated throughout the duration of sleep, usually four or five times. The cyclical nature of sleep is regular and reliable; a REM period occurs about every 90 minutes during the night. However, the first REM period tends to be the shortest, often lasting less than 10 minutes, whereas the later REM periods may last up to 40 minutes. With aging, the time between retiring and sleep onset increases and the total amount of night-time sleep decreases because of changes in sleep architecture that impair sleep maintenance as well as sleep quality. Both NREM (particularly stages 3 and 4) and REM sleep are reduced. However, stage 1 NREM sleep, which is the lightest sleep, increases with age.

As used herein, the term “delta power” means a measure of the duration of electroencephalogram (EEG) activity in the 0.5 to 3.5 Hz range during NREM sleep and is thought to be a measure of deeper, more refreshing sleep. Delta power is hypothesized to be a measure of a theoretical process called Process S and is thought to be inversely related to the amount of sleep an individual experiences during a given sleep period. Sleep is controlled by homeostatic mechanisms; therefore, the less one sleeps the greater the drive to sleep. It is believed that Process S builds throughout the wake period and is discharged most efficiently during delta power sleep. Delta power is a measure of the magnitude of Process S prior to the sleep period. The longer one stays awake, the greater Process S or drive to sleep and thus the greater the delta power during NREM sleep. However, individuals with sleep disorders have difficulty achieving and maintaining delta wave sleep, and thus have a large build-up of Process S with limited ability to discharge this buildup during sleep.

Subjective and Objective Determinations of Sleep Disorders

There are a number of ways to determine whether the onset, duration or quality of sleep (e.g. non-restorative or restorative sleep) is impaired or improved. One method is a subjective determination of the patient, e.g., do they feel drowsy or rested upon waking. Other methods involve the observation of the patient by another during sleep, e.g., how long it takes the patient to fall asleep, how many times does the patient wake up during the night, how restless is the patient during sleep, etc. Another method is to objectively measure the stages of sleep using polysomnography.

Polysomnography is the monitoring of multiple electrophysiological parameters during sleep and generally includes measurement of electroencephalogram (EEG) activity, electroculographic (EOG) activity and electromyographic (EMG) activity, as well as other measurements for the purpose of recording sleep architecture. These results, along with observations, can measure not only sleep latency (the amount of time required to fall asleep), but also sleep continuity (overall balance of sleep and wakefulness) and sleep consolidation (percent of sleeping time spent in delta-wave or restorative sleep) which may be an indication of the quality of sleep.

There are five distinct sleep stages, which can be measured by polysomnography: rapid eye movement (REM) sleep and four stages of non-rapid eye movement (NREM) sleep (stages 1, 2, 3 and 4). Stage 1 NREM sleep is a transition from wakefulness to sleep and occupies about 5% of time spent asleep in healthy adults. Stage 2 NREM sleep, which is characterized by specific EEG waveforms (sleep spindles and K complexes), occupies about 50% of time spent asleep. Stages 3 and 4 NREM sleep (also known collectively as slow-wave sleep and delta-wave sleep) are the deepest levels of sleep and occupy about 10-20% of sleep time. REM sleep, during which the majority of vivid dreams occur, occupies about 20-25% of total sleep.

These sleep stages have a characteristic temporal organization across the night. NREM stages 3 and 4 tend to occur in the first one-third to one-half of the night and increase in duration in response to sleep deprivation. REM sleep occurs cyclically through the night. Alternating with NREM sleep about every 80-100 minutes. REM sleep periods increase in duration toward the morning. Human sleep also varies characteristically across the life span. After relative stability with large amounts of slow-wave sleep in childhood and early adolescence, sleep continuity and depth deteriorate across the adult age range. This deterioration is reflected by increased wakefulness and stage 1 sleep and decreased stages 3 and 4 sleep.

Sleep Disorders

Compounds of the present invention are modulators of the BRS-3 receptor and are useful for the treatment of Sleep Disorders. Inverse agonists and antagonists of the invention are useful for promoting sleep and are useful for promoting one or more of the following: reducing the sleep onset latency period (measure of sleep induction), reducing the number of nighttime awakenings, and prolonging the amount of time in delta-wave sleep (measure of sleep quality enhancement and sleep consolidation) without affecting REM sleep. Inverse agonists and antagonists of the invention are useful as therapeutic agents for promoting sleep and for preventing or treating disorders ameliorated by promoting sleep including, but not limited to, insomnia and the like. Agonists and partial agonists of the invention are useful for the treatment of sleep disorders characterized by excessive sleepiness. Agonists and partial agonists of the invention are useful as therapeutic agents for promoting wakefulness and for preventing or treating excessive sleepiness, such as excessive sleepiness associated with narcolepsy and the like. Accordingly, an aspect of the present invention relates to the therapeutic use of compounds of the present invention for the treatment of Sleep Disorders.

### H. Pharmaceutical Compositions

Compounds of the invention can be formulated into pharmaceutical compositions using techniques well known in the art.

The invention provides methods of treatment (and prevention) by administration to a subject in need of said treatment (or prevention) a therapeutically effect amount of a modulator or a ligand of the invention [also see, e.g., PCT Application Number PCT/IB02/01461 published as WO 02/066505 on 29 Aug. 2002; the disclosure of which is herein incorporated by reference in its entirety]. In one aspect, the modulator or the ligand is a small molecule. In one aspect, the modulator is an inverse agonist or an antagonist. In one aspect, the modulator is an inverse agonist. In one aspect, the modulator is an antagonist. In one aspect, the modulator is substantially purified. In one aspect, the subject is a mammal including, but not limited to cows, pigs, horses, non-human primates, cats, dogs, rabbits, rats, mice, etc., and is preferably a human.

Modulators of the invention can be administered to non-human mammals [see Examples, infra] and/or humans, alone or in pharmaceutical compositions where they are mixed with suitable carriers or excipient(s) using techniques well known to those in the art. Suitable pharmaceutically-acceptable carriers are available to those in the art; for example, see Remington's Pharmaceutical Sciences, 16^{th} Edition, 1980, Mack Publishing Co., (Oslo et al., eds.).

The pharmaceutical composition is then provided at a therapeutically effective dose. A therapeutically effective dose refers to that amount of a modulator sufficient to result in prevention or amelioration of symptoms or physiological status of a disorder as determined illustratively and not by limitation by the methods described herein. In certain embodiments, a therapeutically effective dose refers to that amount of an inverse agonist or antagonist of a mammalian BRS-3 sufficient to result in promotion of sleep. In certain embodiments, a therapeutically effective dose refers to that amount of an agonist or partial agonist of a mammalian BRS-3 sufficient to result in promotion of wakefulness.

It is expressly considered that the modulators of the invention may be provided alone or in combination with other pharmaceutically or physiologically acceptable compounds. In certain embodiments, inverse agonists and antagonists of a mammalian BRS-3 may be provided alone or in combination with other pharmaceutically or physiologically acceptable compounds for the treatment of sleep disorders ameliorated by promoting sleep. In certain embodiments, agonists and partial agonists of a mammalian BRS-3 may be provided alone or in combination with other pharmaceutically or physiologically acceptable compounds for the treatment of excessive sleepiness. In certain embodiments, the excessive sleepiness is associated with a sleep disorder. In certain embodiments, the excessive sleepiness is associated with a neurological disorder. In certain embodiments, said pharmaceutically or physiologically acceptable compound is a compound that binds to a GABA receptor. In certain embodiments, said pharmaceutically or physiologically acceptable compound is a compound that binds to the benzodiazepine binding site on a GABA receptor. In certain embodiments, said pharmaceutically or physiologically acceptable compound is a compound that binds to the benzodiazepine binding site on a GABA_{A} receptor. In certain embodiments, said compound that binds to the benzodiazepine binding site on a GABA receptor, such as a GABA_{A} receptor, allosterically enhances the GABA-evoked chloride flux. In certain embodiments, said compound that binds to the benzodiazepine binding site on a GABA receptor, such as a GABA_{A} receptor, allosterically reduces the GABA-evoked chloride flux. Compounds that bind to the benzodiazepine bind site on a GABA receptor, such as a GABA_{A} receptor, and allosterically enhance or reduce the GABA-evoked chloride flux are known in the art (see, e.g., Da Settimo et al, Curr Med Chem (2007) 14:2680-2701).

It is expressly considered that the modulators of the invention may be provided alone or in combination with other pharmaceutically or physiologically acceptable compounds. In certain embodiments, inverse agonists and antagonists of a mammalian BRS-3 may be provided alone or in combination with other pharmaceutically or physiologically acceptable compounds for the treatment of a GABA-related neurological disorder which is a Sleep Disorder ameliorated by promoting sleep (e.g., Insomnia), an Anxiety Disorder (e.g., Generalized Anxiety Disorder or Panic Attack), a Convulsive Disorder (e.g., Epilepsy), Migraine, a Depressive Disorder (e.g., Major Depressive Disorder), or a Psychotic Disorder (e.g., Schizophrenia). In certain embodiments, agonists and partial agonists of a mammalian BRS-3 may be provided alone or in combination with other pharmaceutically or physiologically acceptable compounds for the treatment of a GABA-related neurological disorder which is a Sleep Disorder ameliorated by promoting wakefulness (e.g., Narcolepsy) or a Cognitive Disorder (e.g., Dementia or Dementia of the Alzheimer's Type). In certain embodiments, said pharmaceutically or physiologically acceptable compound is a compound that binds to a GABA receptor. In certain embodiments, said pharmaceutically or physiologically acceptable compound is a compound that binds to the benzodiazepine binding site on a GABA receptor. In certain embodiments, said pharmaceutically or physiologically acceptable compound is a compound that binds to the benzodiazepine binding site on a GABA_{A} receptor. In certain embodiments, said compound that binds to the benzodiazepine binding site on a GABA receptor, such as a GABA_{A} receptor, allosterically enhances the GABA-evoked chloride flux. In certain embodiments, said compound that binds to the benzodiazepine binding site on a GABA receptor, such as a GABA_{A} receptor, allosterically reduces the GABA-evoked chloride flux. Compounds that bind to the benzodiazepine bind site on a GABA receptor, such as a GABA_{A} receptor, and allosterically enhance or reduce the GABA-evoked chloride flux are known in the art (see, e.g., Da Settimo et al, Curr Med Chem (2007) 14:2680-2701).

While the compounds of the invention can be administered as the sole active pharmaceutical agent (i.e., mono-therapy), compounds of the invention can also be used in combination with other pharmaceutical agents (i.e., combination-therapy) for the treatment of the diseases/conditions/disorders described herein. Therefore, another aspect of the present invention includes methods of treatment comprising administering to a subject in need of treatment (e.g., for promoting sleep or for preventing or treating a sleep disorder ameliorated by promoting sleep) a therapeutically effective amount of an antagonist or an inverse agonist of the present invention in combination with one or more additional pharmaceutical agent as described herein. A further aspect of the present invention includes methods of treatment (e.g., for promoting wakefulness or for preventing or treating excessive sleepiness) comprising administering to a subject in need of treatment a therapeutically effective amount of an agonist or a partial agonist of the present invention in combination with one or more additional pharmaceutical agent as described herein.

It will be understood that the scope of combination-therapy of the compounds of the present invention with other pharmaceutical agents is not limited to those listed herein, supra or infra, but includes in principle any combination with any pharmaceutical agent or pharmaceutical composition useful for the treatment of diseases, conditions or disorders of the present invention in a subject.

Routes of Administration

Suitable routes of administration include oral, nasal, rectal, transmucosal, transdermal, or intestinal administration, parenteral delivery, including intramuscular, subcutaneous, intramedullary injections, as well as intrathecal, direct intraventricular, intravenous, intraperitoneal, intranasal, intrapulmonary (inhaled) or intraocular injections using methods known in the art. Other suitable routes of administration are aerosol and depot formulation. Sustained release formulations, particularly depot, of the invented medicaments are expressly contemplated. In certain embodiments, route of administration is oral.

Composition/Formulation

Pharmaceutical or physiologically acceptable compositions and medicaments for use in accordance with the present invention may be formulated in a conventional manner using one or more physiologically acceptable carriers comprising excipients and auxiliaries. Proper formulation is dependent upon the route of administration chosen.

Certain of the medicaments described herein will include a pharmaceutically or physiologically acceptable carrier and at least one modulator of the invention. For injection, the agents of the invention may be formulated in aqueous solutions, preferably in physiologically compatible buffers such as Hanks's solution, Ringer's solution, or physiological saline buffer such as a phosphate or bicarbonate buffer. For transmucosal administration, penetrants appropriate to the barrier to be permeated are used in the formulation. Such penetrants are generally known in the art.

Pharmaceutical or physiologically acceptable preparations that can be taken orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs for a nebulizer, with the use of a suitable gaseous propellant, e.g., carbon dioxide. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage for, e.g., in ampoules or in multi-dose containers, with an added preservative. The compositions may take such forms as suspension, solutions or emulsions in aqueous vehicles, and may contain formulatory agents such as suspending, stabilizing and/or dispersing agents.

Pharmaceutical or physiologically acceptable formulations for parenteral administration include aqueous solutions of the active compounds in water-soluble form. Aqueous suspension may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder or lyophilized form for constitution with a suitable vehicle, such as sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In a particular embodiment, the compounds can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321:574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, New York, 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). Other controlled release systems are discussed in the review by Langer (1990, Science 249:1527-1533).

Additionally, the compounds may be delivered using a sustained-release system, such as semipermeable matrices of solid hydrophobic polymers containing the therapeutic agent. Various sustained release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for modulator stabilization may be employed.

The pharmaceutical or physiologically acceptable compositions also may comprise suitable solid or gel phase carriers or excipients. Examples of such carriers or excipients include but are not limited to calcium carbonate, calcium phosphate, various sugars, starches, cellulos derivatives, gelatin, and polymers such as polyethylene glycols.

Effective Dosage

Pharmaceutical or physiologically acceptable compositions suitable for use in the present invention include compositions wherein the active ingredients are contained in an effective amount to achieve their intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes or encompasses a concentration point or range shown to increase or decrease an intracellular level of IP3 in a cell comprising BRS-3 in an in vitro assay. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD₅₀ (the dose lethal to 50% of the test population) and the ED₅₀ (the dose therapeutically effective in 50% of the test population). The dose ratio between toxic and therapeutic effects is the therapeutic index and it can be expressed as the ratio between LD₅₀ and ED₅₀. Compounds that exhibit high therapeutic indices are preferred.

The data obtained from these cell culture assays and animal studies can be used in formulating a range of dosage for use in humans. The dosage of such compounds lies preferably within a range of circulating concentrations that include the ED₅₀, with little or no toxicity. The dosage may vary within this range depending upon the dosage form employed and the route of administration utilized. The exact formulation, route of administration and dosage can be chosen by the subject physician in view of the patient's condition. (See, e.g., Fingl et al., 1975, in “The Pharmacological Basis of Therapeutics”, Ch. 1).

Dosage amount and interval may be adjusted subjectly to provide plasma levels of the active compound which are sufficient to prevent or treat a disorder of the invention, depending on the particular situation. Dosages necessary to achieve these effects will depend on subject characteristics and route of administration.

Dosage intervals can also be determined using the value for the minimum effective concentration. Compounds should be administered using a regimen that maintains plasma levels above the minimum effective concentration for 10-90% of the time, preferably between 30-99%, and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration, and the judgement of the prescribing physician.

A preferred dosage range for the amount of a modulator of the invention, which can be administered on a daily or regular basis to achieve desired results is 0.1-100 mg/kg body mass. Other preferred dosage range is 0.1-30 mg/kg body mass. Other preferred dosage range is 0.1-10 mg/kg body mass. Other preferred dosage range is 0.1-3.0 mg/kg body mass. Of course, these daily dosages can be delivered or administered in small amounts periodically during the course of a day. It is noted that these dosage ranges are only preferred ranges and are not meant to be limiting to the invention. Said desired results include, but are not limited to, promotion of sleep or promotion of wakefulness or therapeutic efficacy for a sleep disorder, an anxiety disorder, a convulsive disorder, migraine, a depressive disorder, a psychotic disorder or a cognitive disorder. Said desired results include, but are not limited to, decreasing body mass in a subject, decreasing adiposity in a subject, decreasing percentage body fat in a subject, and preventing or treating obesity or a condition related thereto.

### I. Methods of Treatment

The invention is drawn inter alia to methods including, but not limited to, methods of promoting sleep or wakefulness and methods of preventing or treating a sleep disorder, comprising administering to a subject in need thereof a modulator of the invention.

In some embodiments, the modulator is an inverse agonist or antagonist of a mammalian BRS-3 for use to promote sleep or to prevent or treat a sleep disorder ameliorated by promoting sleep. In some embodiments, the modulator is an inverse agonist or antagonist for use to promote sleep consolidation. In some embodiments, the modulator is an inverse agonist or antagonist for use to increase delta power. In some embodiments, the sleep disorder comprises fragmented sleep architecture. In some embodiments, the sleep disorder ameliorated by promoting sleep is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder. It is expressly contemplated that said sleep disorders ameliorated by promoting sleep may be included in embodiments of the present invention individually or in any combination. In some embodiments, the sleep disorder ameliorated by promoting sleep is insomnia. In some embodiments, the sleep disorder ameliorated by promoting sleep is shift work sleep disorder. In some embodiments, the sleep disorder ameliorated by promoting sleep is time zone change (jet lag) syndrome. In some embodiments, the sleep disorder ameliorated by promoting sleep is obstructive sleep apnea syndrome. In some embodiments, the modulator is an inverse agonist or antagonist of a mammalian BRS-3 for use to promote sleep or to prevent or treat a sleep disorder ameliorated by promoting sleep, with the proviso that the sleep disorder ameliorated by promoting sleep is not obstructive sleep apnea syndrome. In some embodiments, the modulator for use to promote sleep or to prevent or treat a sleep disorder ameliorated by promoting sleep is an inverse agonist of a mammalian BRS-3. In some embodiments, the modulator for use to promote sleep or to prevent or treat a sleep disorder ameliorated by promoting sleep is an antagonist of a mammalian BRS-3.

In some embodiments, the modulator is an inverse agonist or antagonist of a mammalian BRS-3 for use to treat a GABA-related neurological disorder which is a Sleep Disorder ameliorated by promoting sleep, an Anxiety Disorder, a Convulsive Disorder, Migraine, a Depressive Disorder, or a Psychotic Disorder. In some embodiments, the Sleep Disorder ameliorated by promoting sleep is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder. In some embodiments, the Sleep Disorder ameliorated by promoting sleep is Insomnia. In some embodiments, the GABA-related neurological disorder or the Sleep Disorder ameliorated by promoting sleep is not obstructive sleep apnea syndrome. In some embodiments, the Anxiety Disorder is selected from the group consisting of Panic Attack, Agoraphobia, Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder, Anxiety Due to a General Medical Condition, Substance-Induced Anxiety Disorder, Separation Anxiety Disorder, Sexual Aversion Disorder, and Anxiety Disorder Not Otherwise Specified. In some embodiments, the Anxiety Disorder is Generalized Anxiety Disorder. In some embodiments, the Anxiety Disorder is Panic Attack. In some embodiments, the Convulsive Disorder is selected from the group consisting of Epilepsy and Non-Epileptic Seizure. In some embodiments, the Convulsive Disorder is Epilepsy. In some embodiments, the Depressive Disorder is selected from the group consisting of Major Depressive Disorder, Dysthymic Disorder, and Depressive Disorder Not Otherwise Specified. In some embodiments, the Depressive Disorder is Major Depressive Disorder. In some embodiments, the Psychotic Disorder is selected from the group consisting of Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, and Psychotic Disorder Not Otherwise Specified. In some embodiments, the Psychotic Disorder is Schizophrenia. In some embodiments, Schizophrenia is selected from Paranoid Schizophrenia, Disorganized Schizophrenia, Catatonic Schizophrenia, Undifferentiated Schizophrenia, and Residual Schizophrenia. In some embodiments, the modulator is an inverse agonist of a mammalian BRS-3. In some embodiments, the modulator is an antagonist of a mammalian BRS-3.

In some embodiments, the modulator is an agonist or a partial agonist of a mammalian BRS-3 for use to promote wakefulness or to prevent or treat excessive sleepiness. In some embodiments, the modulator is an agonist or a partial agonist for use to prevent or treat excessive sleepiness associated with a sleep disorder. In some embodiments, the modulator is an agonist or a partial agonist for use to prevent or treat excessive sleepiness associated with a sleep disorder selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder. In some embodiments, the modulator is an agonist or a partial agonist for use to prevent or treat excessive sleepiness associated with narcolepsy. It is expressly contemplated that said sleep disorder having association with excessive sleepiness may be included in embodiments of the invention individually or in any combination. In some embodiments, the modulator is an agonist or a partial agonist for use to prevent or treat excessive sleepiness associated with shift work sleep disorder. In some embodiments, the modulator is an agonist or a partial agonist for use to prevent or treat excessive sleepiness associated with time zone change (jet lag) syndrome. In some embodiments, the modulator is an agonist or a partial agonist for use to prevent or treat excessive sleepiness associated with obstructive sleep apnea syndrome. In some embodiments, the modulator is an agonist or a partial agonist of a mammalian BRS-3 for use to promote wakefulness or to prevent or treat excessive sleepiness associated with a sleep disorder, with the proviso that the sleep disorder is not obstructive sleep apnea syndrome. In some embodiments, the modulator is an agonist or a partial agonist for use to prevent or treat excessive sleepiness associated with a neurological disorder. In some embodiments, the modulator is an agonist or a partial agonist for use to prevent or treat excessive sleepiness associated with a neurological disorder selected from the group consisting of multiple sclerosis, myonic dystrophy, and Parkinson's disease. In some embodiments, the modulator is an agonist or a partial agonist for use to prevent or treat excessive sleepiness associated with a psychiatric disorder. In some embodiments, the modulator is an agonist or a partial agonist for use to prevent or treat excessive sleepiness associated with a psychiatric disorder selected from the group consisting of depression and schizophrenia. In some embodiments, the modulator is an agonist or a partial agonist for use to prevent or treat excessive sleepiness, wherein the excessive sleepiness is hypersomnia. In some embodiments, the modulator for use to promote wakefulness or to prevent or treat excessive sleepiness is an agonist of a mammalian BRS-3. In some embodiments, the modulator for use to promote wakefulness or to prevent or treat excessive sleepiness is a partial agonist of a mammalian BRS-3.

In some embodiments, the modulator is an agonist or a partial agonist of a mammalian BRS-3 for use to treat a GABA-related neurological disorder which is a Sleep Disorder ameliorated by promoting wakefulness or a Cognitive Disorder. In some embodiments, the Sleep Disorder ameliorated by promoting wakefulness is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder. In some embodiments, the GABA-related neurological disorder or the Sleep Disorder ameliorated by promoting wakefulness is Narcolepsy. In some embodiments, the GABA-related neurological disorder or the Sleep Disorder ameliorated by promoting wakefulness is not obstructive sleep apnea syndrome. In some embodiments, the GABA-related neurological disorder is a Cognitive Disorder. In some embodiments, the Cognitive Disorder is selected from the group consisting of Delirium, Dementia, Amnestic Disorder, and Cognitive Disorder Not Otherwise Specified. In some embodiments, Delirium is selected from the group consisting of Delirium Due to a General Medical Condition, Substance-Induced Delirium, Delirium Due to Multiple Etiologies, and Delirium Not Otherwise Specified. In some embodiments, Dementia is selected from the group consisting of Dementia of the Alzheimer's Type, Vascular Dementia, Dementia Due to Other General Medical Conditions, Substance-Induced Persisting Dementia, Dementia Due to Multiple Etiologies, and Dementia Not Otherwise Specified. In some embodiments, Amnestic Disorder is selected from the group consisting of Amnestic Disorder Due to a General Medical Condition, Substance-Induced Persisting Amnestic Disorder, and Amnestic. Disorder Not Otherwise Specified. In some embodiments, the Cognitive Disorder is Dementia. In some embodiments, the Cognitive Disorder is Dementia of the Alzheimer's Type. In some embodiments, the modulator is an agonist of a mammalian BRS-3. In some embodiments, the modulator is a partial agonist of a mammalian BRS-3.

It is expressly contemplated that each of the GABA-related neurological disorders set forth in the present application, as well as each combination of said GABA-related neurological disorders, is a separate embodiment within the scope of the present invention. It is expressly contemplated that each of the Sleep Disorders set forth in the present application, as well as each combination of said Sleep Disorders, is a separate embodiment within the scope of the present invention. It is expressly contemplated that each of the Sleep Disorders ameliorated by promoting sleep set forth in the present application, as well as each combination of said Sleep Disorders ameliorated by promoting sleep, is a separate embodiment within the scope of the present invention. It is expressly contemplated that each of the Anxiety Disorders set forth in the present application, as well as each combination of said Anxiety Disorders, is a separate embodiment within the scope of the present invention. It is expressly contemplated that each of the Convulsive Disorders set forth in the present application, as well as each combination of said Convulsive Disorders, is a separate embodiment within the scope of the present invention. It is expressly contemplated that each of the Depressive Disorders set forth in the present application, as well as each combination of said Depressive Disorders, is a separate embodiment within the scope of the present invention. It is expressly contemplated that each of the Psychotic Disorders set forth in the present application, as well as each combination of said Psychotic Disorders, is a separate embodiment within the scope of the present invention. It is expressly contemplated that each of the Schizophrenia subtypes set forth in the present application, as well as each combination of said Schizophrenia subtypes, is a separate embodiment within the scope of the present invention. It is expressly contemplated that each of the Sleep Disorders ameliorated by promoting wakefulness set forth in the present application, as well as each combination of said Sleep Disorders ameliorated by promoting wakefulness, is a separate embodiment within the scope of the present invention. It is expressly contemplated that each of the Cognitive Disorders set forth in the present application, as well as each combination of said Cognitive Disorders, is a separate embodiment within the scope of the present invention.

In some embodiments, the modulator is orally active. In some embodiments, said orally active modulator is further able to cross the blood-brain barrier. In some embodiments, the modulator is administered to the subject in a pharmaceutical composition. In some embodiments, the modulator is provided to the subject in a pharmaceutical composition. In some embodiments, the modulator is provided to the subject in a pharmaceutical composition that is taken orally. In some embodiments, the subject is a non-human mammal. In some embodiments, the subject is a mammal. In certain embodiments, the mammal is a mouse, a rat, a non-human primate, or a human. In certain preferred embodiments, the subject or mammal is a human.

### J. Other Utility

Agents that modulate (i.e., increase, decrease, or block) receptor functionality of a GPCR of the invention such as a mammalian BRS-3 receptor may be identified by contacting a candidate compound with the GPCR and determining the effect of the candidate compound on receptor functionality. The selectivity of a compound that modulates the functionality of a mammalian BRS-3 receptor such as human BRS-3 receptor can be evaluated by comparing its effects on BRS-3 to its effects on one or more other G protein-coupled receptors. In certain embodiments, a BRS-3 selective modulator is a BRS-3 selective inverse agonist or antagonist having a selectivity for BRS-3 over gastric-releasing peptide receptor (GRP-R; e.g., human GRP-R, GenBank® Accession No. NP_{—}005305) or neuromedin B receptor (NMB-R; e.g., human NMB-R, GenBank® Accession No. NP_{—}002502) of at least about 10-fold or of at least about 100-fold. In certain embodiments, a BRS-3 selective modulator is a BRS-3 selective inverse agonist or antagonist having a selectivity for BRS-3 over GRP-R and NMB-R of at least about 10-fold or of at least about 100-fold. In certain embodiments, a BRS-3 selective modulator is a BRS-3 selective agonist or partial agonist having a selectivity for BRS-3 over GRP-R or NMB-R of at least about 10-fold or of at least about 100-fold. In certain embodiments, a BRS-3 selective modulator is a BRS-3 selective agonist or partial agonist having a selectivity for BRS-3 over GRP-R and NMB-R of at least about 10-fold or of at least about 100-fold. Following identification of compounds that modulate BRS-3 functionality, such candidate compounds may be further tested in other assays including, but not limited to, in vivo models, in order to confirm or quantitate their activity. By way of illustration and not limitation, the subject invention expressly contemplates the identification of compounds as modulators of a mammalian BRS-3 GPCR for use as pharmaceutical agents. Modulators of BRS-3 receptor functionality are therapeutically useful, e.g., in treatment of diseases and physiological conditions in which normal or aberrant BRS-3 functionality is involved.

Agents that are ligands of a GPCR of the invention such as a mammalian BRS-3 receptor may be identified by contacting a candidate compound with the GPCR and determining whether the candidate compound binds to the receptor. The selectivity of a compound that binds to a mammalian BRS-3 receptor such as human BRS-3 receptor can be evaluated by comparing its binding to BRS-3 receptor to its binding to one or more other G protein-coupled receptors. In certain embodiments, a BRS-3 selective ligand is a BRS-3 selective ligand having a selectivity for BRS-3 over gastric-releasing peptide receptor (GRP-R; e.g., human GRP-R, GenBank® Accession No. NP_{—}005305) or neuromedin B receptor (NMB-R; e.g., human NMB-R, GenBank® Accession No. NP_{—}002502) of at least about 10-fold or of at least about 100-fold. In certain embodiments, a BRS-3 selective ligand is a BRS-3 selective ligand having a selectivity for BRS-3 over GRP-R and NMB-R of at least about 10-fold or of at least about 100-fold. Ligands that are modulators of BRS-3 receptor functionality are therapeutically useful in treatment of diseases, disorders and physiological conditions in which normal or aberrant BRS-3 functionality is involved.

In other embodiments, agents that are suitable for, e.g., promoting sleep or for promoting wakefulness or that are useful as pharmaceutical agents for, e.g., sleep disorders are identified by contacting a candidate compound with a BRS-3 receptor and determining the effect of the candidate compound on BRS-3 receptor expression. In some embodiments, the agent reduces expression of BRS-3 receptor in a cell. In some embodiments, the agent reduces expression of BRS-3 receptor in a neuronal cell. In some embodiments, the agent reduces expression of BRS-3 receptor in a human neuronal cell. In some embodiments, the BRS-3 receptor is endogenously expressed by the cell or neuronal cell. In some embodiments, a level of BRS-3 receptor expression is measured using anti-BRS-3 receptor antibody. In some embodiments, a level of BRS-3 receptor expression is measured using anti-BRS-3 receptor antibody by immunohistochemistry or flow cytometry or Western blot. It is expressly contemplated that the anti-BRS-3 antibody can be monoclonal or polyclonal. Antibodies to BRS-3 are commercially available; for example, antibody to human BRS-3 is available from Atlas Antibodies (Stockholm, Sweden) and from ABR-Affinity BioReagents (Golden, Colo.). In some embodiments, a level of BRS-3 receptor expression is measured using radiolableled ligand specific for BRS-3 receptor (see infra). In some embodiments, a level of BRS-3 receptor expression is measured by in situ hybridization or Northern blot or RT-PCR.

The present invention also relates to a method for identifying compounds suitable for promoting sleep or for preventing or treating a sleep disorder ameliorated by promoting sleep or for a GABA-related neurological disorder selected from the group consisting of a sleep disorder ameliorated by promoting sleep, an anxiety disorder, a convulsive disorder, migraine, a depressive disorder and a psychotic disorder, said method comprising the steps of:
(a″) contacting or not contacting a plurality of cells comprising a BRS-3 receptor with a candidate compound;
(b″) measuring the level of expression of the BRS-3 receptor in the cells contacted with the candidate compound and the level of expression of the BRS-3 receptor in the cells not contacted with the candidate compound; and
(c″) comparing the level of expression of the BRS-3 receptor in the cells contacted with the candidate compound with the level of expression of the BRS-3 receptor in the cells not contacted with the candidate compound;
wherein a decrease in the level of expression of the BRS-3 receptor in the cells contacted with the candidate compound compared with the level of expression of the BRS-3 receptor in the cells not contacted with the candidate compound is indicative of the candidate compound being a compound suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder and a psychotic disorder.

The invention additionally features a method for identifying compounds suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder and a psychotic disorder, said method comprising steps (a″) to (c″) supra and further comprising:
(d″) optionally synthesizing a compound in the presence of which the level of expression of the BRS-3 receptor is decreased in step (c″);
(e″) administering a compound in the presence of which the level of expression of the BRS-3 receptor is decreased in step (c″) to a mammal; and
(f″) determining whether the compound promotes sleep, has anxiolytic activity, has anticonvulsant activity, has anti-migraine activity, has antidepressant activity, or has antipsychotic activity in the mammal;
wherein the ability of the candidate compound to promote sleep, to show anxiolytic activity, to show anticonvulsant activity, to show anti-migraine activity, to show antidepressant activity, or to show antipsychotic activity in the mammal is indicative of the candidate compound being a compound suitable for promoting sleep or for preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder and a psychotic disorder.

In some embodiments, the mammal is a non-human mammal. In some embodiments, the non-human mammal is a laboratory animal. In some embodiments, the non-human mammal is a non-human primate. In some embodiments, the non-human mammal is a rodent. In some embodiments, the non-human mammal is a rat. In some embodiments, the non-human mammal is a mouse.

In some embodiments, said determining whether the compound promotes sleep in the mammal comprises polysomnography.

In some embodiments, said method of identifying whether a candidate compound is an agent that decreases expression of a BRS-3 receptor in a cell is an in vitro method.

In some embodiments, said plurality of cells contacted or not contacted with the candidate compound in step (a″) are cultured for at least about 1 hour, at least about 2 hours, at least about 4 hours, at least about 8 hours, at least about 16 hours, at least about 24 hours, at least about 36 hours or at least about 48 hours before the level of expression of the BRS-3 receptor in said cells is measured in step (b″).

The present invention relates to said compound that decreases BRS-3 expression in a cell (e.g., a neuronal cell), to a composition comprising said compound (e.g., a pharmaceutical composition), and to methods of using said composition (e.g., for promoting sleep or for preventing or treating a sleep disorder ameliorated by promoting sleep), wherein the compound is a small molecule. The present invention relates to said compound that decreases BRS-3 expression in a cell (e.g., a neuronal cell), to a composition comprising said compound (e.g., a pharmaceutical composition), and to methods of using said composition (e.g., for promoting sleep or for preventing or treating a sleep disorder ameliorated by promoting sleep), wherein the compound is antisense nucleic acid (e.g., antisense RNA). The present invention relates to said compound that decreases BRS-3 expression in a cell (e.g., a neuronal cell), to a composition comprising said compound (e.g., a pharmaceutical composition), and to methods of using said composition (e.g., for promoting sleep or for preventing or treating a sleep disorder ameliorated by promoting sleep), wherein the compound is a small interfering RNA (siRNA) or short hairpin RNA (shRNA) molecule comprising a nucleotide sequence derived from the nucleotide sequence of a BRS-3 receptor-encoding gene according to standard procedures. As will be known to the skilled artisan, siRNA, shRNA and antisense RNA are generally capable of modulating expression of a target gene [see, e.g., Holmlund J T, Ann NY Acad Sci (2003) 1002:244-251; and Devroe et al, Expert Opin Biol Ther (2004) 4:319-327; the disclosure of each of which is hereby incorporated by reference in its entirety].

The present invention also relates to a method for identifying compounds suitable for promoting wakefulness or for preventing or treating excessive sleepiness or for preventing or treating a GABA-related neurological disorder selected from the group consisting of a sleep disorder ameliorated by promoting wakefulness and a cognitive disorder, said method comprising the steps of:
(a′″) contacting or not contacting a plurality of cells comprising a BRS-3 receptor with a candidate compound;
(b′″) measuring the level of expression of the BRS-3 receptor in the cells contacted with the candidate compound and the level of expression of the BRS-3 receptor in the cells not contacted with the candidate compound; and
(c′″) comparing the level of expression of the BRS-3 receptor in the cells contacted with the candidate compound with the level of expression of the BRS-3 receptor in the cells not contacted with the candidate compound;
wherein an increase in the level of expression of the BRS-3 receptor in the cells contacted with the candidate compound compared with the level of expression of the BRS-3 receptor in the cells not contacted with the candidate compound is indicative of the candidate compound being a compound suitable for promoting wakefulness or for preventing or treating excessive sleepiness or for preventing or treating a GABA-related neurological disorder selected from the group consisting of a sleep disorder ameliorated by promoting wakefulness and a cognitive disorder.

The invention additionally features a method for identifying compounds suitable for promoting wakefulness or for preventing or treating excessive sleepiness or for preventing or treating a GABA-related neurological disorder selected from the group consisting of a sleep disorder ameliorated by promoting wakefulness and a cognitive disorder, said method comprising steps (a′″) to (c′″) supra and further comprising:
(d′″) optionally synthesizing a compound in the presence of which the level of expression of the BRS-3 receptor is increased in step (c′″);
(e′″) administering a compound in the presence of which the level of expression of the BRS-3 receptor is increased in step (c′″) to a mammal; and
(f″) determining whether the compound promotes wakefulness or has cognition-enhancing activity in the mammal;
wherein the ability of the candidate compound to promote wakefulness or to show cognition-enhancing activity in the mammal is indicative of the candidate compound being a compound suitable for promoting wakefulness or for preventing or treating excessive sleepiness or for preventing or treating a GABA-related neurological disorder selected from the group consisting of a sleep disorder ameliorated by promoting wakefulness and a cognitive disorder.

In some embodiments, the mammal is a non-human mammal. In some embodiments, the non-human mammal is a laboratory animal. In some embodiments, the non-human mammal is a non-human primate. In some embodiments, the non-human mammal is a rodent. In some embodiments, the non-human mammal is a rat. In some embodiments, the non-human mammal is a mouse.

In some embodiments, said determining whether the compound promotes wakefulness in the mammal comprises polysomnography.

In some embodiments, said method of identifying whether a candidate compound is an agent that increases expression of a BRS-3 receptor in a cell is an in vitro method.

In some embodiments, said plurality of cells contacted or not contacted with the candidate compound in step (a′″) are cultured for at least about 1 hour, at least about 2 hours, at least about 4 hours, at least about 8 hours, at least about 16 hours, at least about 24 hours, at least about 36 hours or at least about 48 hours before the level of expression of the BRS-3 receptor in said cells is measured in step (b′″).

The present invention relates to said compound that increases BRS-3 expression in a cell (e.g., a neuronal cell), to a composition comprising said compound (e.g., a pharmaceutical composition), and to methods of using said composition (e.g., for promoting sleep or for preventing or treating a sleep disorder ameliorated by promoting sleep), wherein the compound is a small molecule.

The present invention also relates to radioisotope-labeled versions of compounds of the invention identified as modulators or ligands of a GPCR of the invention such as a mammalian BRS-3 that would be useful not only in radio-imaging but also in assays, both in vitro and in vivo, for localizing and quantitating BRS-3 in tissue samples, including human, and for identifying BRS-3 ligands in methods relating to inhibition of binding of a radioisotope-labeled compound such as a known ligand of BRS-3. It is a further object of this invention to develop novel assays relating to a GPCR of the invention such as a mammalian BRS-3, such as human BRS-3, which comprise such radioisotope-labeled compounds. By way of illustration and not limitation, it is envisioned that increased brain BRS-3 above a normal range visualized by radio-imaging identifies a subject at risk for a sleep disorder ameliorated by the promotion of sleep, such as insomnia and the like, for an anxiety disorder, such as Generalized Anxiety Disorder and Panic Attack and the like, for a convulsive disorder, such as Epilepsy and the like, for migraine, for a Depressive Disorder such as Major Depressive Disorder and the like, or for a psychotic disorder such as Schizophrenia and the like. Also by way of illustration and not limitation, it is envisioned that decreased brain BRS-3 below a normal range visualized by radio-imaging identifies a subject at risk for a sleep disorder ameliorated by the promotion of wakefulness, such as hypersomnia, narcolepsy and the like, or for a cognitive disorder, such as Dementia and Dementia of the Alzheimer's Type and the like. In some embodiments, the brain BRS-3 is hypothalamic BRS-3. In some embodiments, the subject is a human.

The present invention also relates a method of radio-imaging comprising administering to a mammal in need of said radio-imaging a radiolabeled compound that is a modulator or a ligand of the mammalian BRS-3 receptor. In one aspect, the ligand of the mammalian BRS-3 receptor is not a modulator of the mammalian BRS-3 receptor. In some embodiments, the mammal is a human. In some embodiments, the method of radio-imaging is for identifying whether the mammal is at risk for or progressing toward a sleep disorder ameliorated by promoting sleep, such as insomnia and the like, for an anxiety disorder, such as Generalized Anxiety Disorder and Panic Attack and the like, for a convulsive disorder, such as Epilepsy and the like, for migraine, for a Depressive Disorder such as Major Depressive Disorder and the like, or for a psychotic disorder such as Schizophrenia and the like, wherein a level of brain BRS-3 in the mammal above the normal range is indicative of the mammal being at risk for or progressing toward a sleep disorder ameliorated by the promotion of sleep, such as insomnia and the like, for an anxiety disorder, such as Generalized Anxiety Disorder and Panic Attack and the like, for a convulsive disorder, such as Epilepsy and the like, for migraine, for a Depressive Disorder such as Major Depressive Disorder and the like, or for a psychotic disorder such as Schizophrenia and the like. In some embodiments, the method of radio-imaging is for identifying the mammal as in need of preventing or treating a sleep disorder ameliorated by promoting sleep, such as insomnia and the like, for an anxiety disorder, such as Generalized Anxiety Disorder and Panic Attack and the like, for a convulsive disorder, such as Epilepsy and the like, for migraine, for a Depressive Disorder such as Major Depressive Disorder and the like, or for a psychotic disorder such as Schizophrenia and the like, with an inverse agonist or an antagonist of the mammalian BRS-3 or with a compound that decreases BRS-3 expression in a cell or with a pharmaceutical composition comprising the inverse agonist or the antagonist or the compound that decreases BRS-3 expression in a cell and a pharmaceutically acceptable carrier, wherein a level of brain BRS-3 in the mammal above a normal range identifies the mammal as in need of preventing or treating a sleep disorder ameliorated by the promoting sleep, such as insomnia and the like, for an anxiety disorder, such as Generalized Anxiety Disorder and Panic Attack and the like, for a convulsive disorder, such as Epilepsy and the like, for migraine, for a Depressive Disorder such as Major Depressive Disorder and the like, or for a psychotic disorder such as Schizophrenia and the like, with the inverse agonist or the antagonist of the mammalian BRS-3 or with the compound that decreases BRS-3 expression in a cell that decreases BRS-3 expression in a cell or with the pharmaceutical composition comprising the inverse agonist or the antagonist or the compound that decreases BRS-3 expression in a cell and a pharmaceutically acceptable carrier. In some embodiments, the method of radio-imaging is for identifying whether the mammal is at risk for or progressing toward excessive sleepiness, such as excessive sleepiness associated with a sleep disorder such as narcolepsy and the like, or for a cognitive disorder, such as Dementia and Dementia of the Alzheimer's Type and the like, wherein a level of brain BRS-3 in the mammal below the normal range is indicative of the mammal being at risk for or progressing toward excessive sleepiness, such as excessive sleepiness associated with a sleep disorder such as narcolepsy and the like, or for a cognitive disorder, such as Dementia and Dementia of the Alzheimer's Type and the like. In some embodiments, the method of radio-imaging is for identifying the mammal as in need of preventing or treating excessive sleepiness, such as excessive sleepiness associated with a sleep disorder such as narcolepsy and the like, or for a cognitive disorder, such as Dementia and Dementia of the Alzheimer's Type and the like, with an agonist or a partial agonist of the mammalian BRS-3 or with an compound that increases BRS-3 expression in a cell or with a pharmaceutical composition comprising the agonist or the partial agonist or the compound that increases BRS-3 expression in a cell and a pharmaceutically acceptable carrier, wherein a level of brain BRS-3 in the mammal below a normal range identifies the mammal as in need of preventing or treating excessive sleepiness, such as excessive sleepiness associated with a sleep disorder such as narcolepsy and the like, or for a cognitive disorder, such as Dementia and Dementia of the Alzheimer's Type and the like, with the agonist or the partial agonist of the mammalian BRS-3 or with the compound that increases BRS-3 expression in a cell that increases BRS-3 expression in a cell or with the pharmaceutical composition comprising the agonist or the partial agonist or the compound that increases BRS-3 expression in a cell and a pharmaceutically acceptable carrier. In some embodiments, the brain BRS-3 is hypothalamic BRS-3. In some embodiments, the hypothalamic BRS-3 is dorsomedial hypothalamic nucleus (DMH) BRS-3.

The present invention embraces radioisotope-labeled versions of compounds of the invention identified as modulators or ligands of a GPCR of the invention such as a mammalian BRS-3, such as human BRS-3.

The present invention also relates to radioisotope-labeled versions of test ligands that are useful for detecting a ligand bound to a GPCR of the invention such as a mammalian BRS-3, such as human BRS-3. In some embodiments, the present invention expressly contemplates a library of said radiolabeled test ligands useful for detecting a ligand bound to a GPCR of the invention such as a mammalian BRS-3, such as human BRS-3. In certain embodiments, said library comprises at least about 10, at least about 10², at least about 10³, at least about 10⁵, or at least about 10⁶ said radiolabeled test compounds. It is a further object of this invention to develop novel assays relating to a GPCR of the invention such as a mammalian BRS-3, such as human BRS-3, which comprise such radioisotope-labeled test ligands.

In some embodiments, a radioisotope-labeled version of a compound is identical to the compound, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (deuterium), ³H (tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro BRS-3 receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ⁷⁵Br, ⁷⁶Br or ⁷⁷Br will generally be most useful. In some embodiments, the radionuclide is selected from the group consisting of ³H, ¹¹C, ¹⁸F, ¹⁴C, ¹²⁵I, ¹²⁴I, ¹³¹I, ³⁵S and ⁸²Br.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (3H) products by treating appropriate precursors with high specific activity methyl iodide (3H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

In some embodiments, a radioisotope-labeled version of a compound is identical to the compound, but for the addition of one or more substituents comprising a radionuclide. In some further embodiments, the compound is a small molecule. In some further embodiments, the compound is a polypeptide. In some further embodiments, the compound is an antibody or an antigen-binding fragment thereof. In some further embodiments, said antibody is monoclonal. Suitable said radionuclide includes but is not limited to ²H (deuterium), ³H (tritium), ¹¹C, ¹³C, 14C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro BRS-3 receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is selected from the group consisting of ³H, ¹¹C, ¹⁸F, ¹⁴C, ¹²⁵I, ¹²⁴I, ¹³¹I, ³⁵S and ⁸²Br.

Methods for adding one or more substituents comprising a radionuclide are within the purview of the skilled artisan and include, but are not limited to, addition of radioisotopic iodine by enzymatic method [Marchalonic J J, Biochemical Journal (1969) 113:299-305; Thorell JI and Johansson B G, Biochimica et Biophysica Acta (1969) 251:363-9; the disclosure of each of which is herein incorporated by reference in its entirety] and or by Chloramine-T/Iodogen/Iodobead methods [Hunter W M and Greenwood F C, Nature (1962) 194:495-6; Greenwood F C et al., Biochemical Journal (1963) 89:114-23; the disclosure of each of which is herein incorporated by reference in its entirety].

Other uses of the disclosed receptors and methods will become apparent to those in the art based upon, inter alfa, a review of this patent document.

### EXAMPLES

The following examples are presented for purposes of elucidation, and not limitation, of the present invention. While specific nucleic acid and amino acid sequences are disclosed herein, those of ordinary skill in the art are credited with the ability to make minor modifications to these sequences while achieving the same or substantially similar results reported below. Such modified approaches are considered within the purview of this disclosure. Without further elaboration, it is believed that one skilled in the art can, using the preceding description, practice the present invention to its fullest extent. The following detailed examples are to be construed as merely illustrative, and not limitations of the preceding disclosure in any way whatsoever. Those skilled in the art will promptly recognize appropriate variations from the procedures.

Recombinant DNA techniques relating to the subject matter of the present invention and well known to those of ordinary skill in the art can be found, e.g, in Maniatis T et al., Molecular Cloning: A Laboratory Manual (1989) Cold Spring Harbor Laboratory; U.S. Pat. No. 6,399,373; and PCT Application Number PCT/IB02/01461 published as WO 02/066505 on 29 Aug. 2002; the disclosure of each of which is herein incorporated by reference in its entirety.

### Example 1

Full-Length Cloning of Endogenous Human BRS-3

Polynucleotide encoding endogenous human BRS-3 was cloned by RT-PCR using the BRS-3 specific primers

5′-ACAGAATTCAGAAGAAATGGCTCAAAGGCA-3′ (SEQ ID NO:3; sense with EcoRI site, ATG as initiation codon) and
5′-CATGGATCCTTGAAAAGCTAGAATCTGTCC-3′ (SEQ ID NO:4; antisense with BamHI site, CTA as antisense of stop codon)
and human uterus cDNA (Clontech) as template. TaqPlus Precision™ DNA polymerase (Stratagene) was used for amplification by the following cycle with step 2 to step 4 repeated 25 times:
94° C., 3 minutes; 94° C., 1 minute; 56° C., 1 minute; 72° C., 1 minute 20 sec; 72° C., 10 minutes.
A 1.23 Kb PCR fragment of predicted size was isolated, digested with EcoRI and BamHI, and cloned into the pCMV expression vector and sequenced using the T7 DNA sequenase kit (Amersham). See, SEQ ID NO:1 for nucleic acid sequence and SEQ ID NO:2 for the deduced amino acid sequence.

### Example 2

### Receptor Expression

Although a variety of cells are available to the art for the expression of proteins, it is most preferred that mammalian cells or melanophores be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells or melanophores. Of the mammalian cells, CHO, COS-7, MCB3901, 293, 293T and HeLa cells are particularly preferred, although the specific mammalian cell utilized can be predicated upon the particular needs of the artisan. See infra as relates to melanophores, including Example 9.

a. Transient Transfection

On day one, 4×10⁶ 293 cells per 10 cm dish are plated out. On day two, two reaction tubes are prepared (the proportions to follow for each tube are per plate): tube A is prepared by mixing 4 μg DNA (e.g., pCMV vector; pCMV vector comprising polynucleotide encoding a GPCR of the invention, etc.) in 0.5 ml serum free DMEM (Gibco BRL); tube B is prepared by mixing 24 μl lipofectamine (Gibco BRL) in 0.5 ml serum free DMEM. Tubes A and B are admixed by inversions (several times), followed by incubation at room temperature for 30-45 min. The admixture is referred to as the “transfection mixture”. Plated 293 cells are washed with 1×PBS, followed by addition of 5 ml serum free DMEM. 1 ml of the transfection mixture is added to the cells, followed by incubation for 4 hrs at 37° C./5% CO₂. The transfection mixture is removed by aspiration, followed by the addition of 10 ml of DMEM/10% Fetal Bovine Serum. Cells are incubated at 37° C./5% CO₂. After 48 hr incubation, cells are harvested and utilized for analysis.

b. Stable Cell Lines

Approximately 12×10⁶ 293 cells are plated on a 15 cm tissue culture plate. Grown in DME High Glucose Medium containing ten percent fetal bovine serum and one percent sodium pyruvate, L-glutamine, and antibiotics. Twenty-four hours following plating of 293 cells (or to ˜80% confluency), the cells are transfected using 12 μg of DNA (e.g., pCMV-neo^{r} vector comprising polynucleotide encoding a GPCR of the invention). The 12 μg of DNA is combined with 60 μl of lipofectamine and 2 ml of DME High Glucose Medium without serum. The medium is aspirated from the plates and the cells are washed once with medium without serum. The DNA, lipofectamine, and medium mixture are added to the plate along with 10 ml of medium without serum. Following incubation at 37° C. for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μm/ml. The transfected cells now undergo selection for positively transfected cells containing the G418 resistance gene. The medium is replaced every four to five days as selection occurs. During selection, cells are grown to create stable pools, or split for stable clonal selection.

### Example 3

### Assays for Determination of GPCR Activation (e.g., Screening Assays)

A variety of approaches are available for assessing activation of a GPCR of interest, or “target” GPCR. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used to screen candidate compounds as modulators of GPCRs. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g, 293 cells expressing a GPCR of the invention; this amount can be adjusted for optimization) and 10 μM GDP (this amount can be changed for optimization) for 1 hour. Wheatgerm agglutinin beads (25 μl; Amersham) are then added and the mixture incubated for another 30 minutes at room temperature. The tubes are then centrifuged at 1500×g for 5 minutes at room temperature and then counted in a scintillation counter.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells are harvested approximately twenty-four to forty-eight hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 min. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer is prepared fresh for screening and contains 50 μl of Stimulation Buffer, 3 μl of test compound (12 μM final assay concentration) and 50 μl cells. Assay Buffer is stored on ice until utilized. The assay, preferably carried out e.g. in a 96-well plate, is initiated by addition of 50 μl of cAMP standards to appropriate wells followed by addition of 50 ul of PBS to wells H-11 and H12. 50 μl of Stimulation Buffer is added to all wells. DMSO (or selected candidate compounds) is added to appropriate wells using a pin tool capable of dispensing 3 μl of compound solution, with a final assay concentration of 12 μM test compound and 100 μl total assay volume. The cells are then added to the wells and incubated for 60 min at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which is contained within each assay plate.

3. Cell-Based cAMP Assay for G1-Coupled Target GPCRs

Thyroid Stimulating Hormone Receptor (TSHR) is a Gs coupled GPCR that causes the accumulation of cAMP upon activation. TSHR will be constitutively activated by mutating amino acid residue 623 (i.e., changing an alanine residue to an isoleucine residue). A G1 coupled receptor is expected to inhibit adenylyl cyclase, and, therefore, decrease the level of cAMP production, which can make assessment of cAMP levels challenging. An effective technique for measuring the decrease in production of cAMP as an indication of activation of a Gi coupled receptor can be accomplished by co-transfecting, most preferably, non-endogenous, constitutively activated TSHR (TSHR-A623I) (or an endogenous, constitutively active Gs coupled receptor) as a “signal enhancer” with a Gi coupled Target GPCR to establish a baseline level of cAMP. The Gi coupled receptor is co-transfected with the signal enhancer, and it is this material that can be used for screening. Such an approach can be utilized to effectively generate a signal when a cAMP assay is used. In some embodiments, this approach is preferably used in the identification of candidate compounds against Gi coupled receptors. It is noted that for a G1 coupled GPCR, when this approach is used, an inverse agonist of the Target GPCR will increase the cAMP signal and an agonist will decrease the cAMP signal.

On day one, 4×10⁶293 cells per 10 cm dish will be plated out. On day two, two reaction tubes will be prepared (the proportions to follow for each tube are per plate): tube A will be prepared by mixing 2 μg DNA of each receptor transfected into the mammalian cells, for a total of 4 μg DNA (e.g., pCMV vector; pCMV vector with mutated TSHR (TSHR-A6231); TSHR-A623I and the Target GPCR, etc.) in 0.5 ml serum free DMEM (Irvine Scientific, Irvine, Calif.); tube B will be prepared by mixing 24 μl lipofectamine (Gibco BRL) in 0.5 ml serum free DMEM. Tubes A and B will then be admixed by inversions (several times), followed by incubation at room temperature for 30-45 min. The admixture is referred to as the “transfection mixture”. Plated 293 cells will be washed with 1×PBS, followed by addition of 5 ml serum free DMEM. 1.0 ml of the transfection mixture will then be added to the cells, followed by incubation for 4 hrs at 37° C./5% CO₂. The transfection mixture will then be removed by aspiration, followed by the addition of 10 ml of DMEM/10% Fetal Bovine Serum. Cells will then be incubated at 37° C./5% CO₂. After approximately 24-48 hr incubation, cells will then be harvested and utilized for analysis.

A Flash Plate™ Adenylyl Cyclase kit (New England Nuclear; Cat. No. SMP004A) is designed for cell-based assays, but can be modified for use with crude plasma membranes depending on the need of the skilled artisan. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty-four to forty-eight hours after transient transfection. Media will be carefully aspirated off and discarded. 10 ml of PBS will be gently added to each dish of cells followed by careful aspiration. 1 ml of Sigma cell dissociation buffer and 3 ml of PBS will be added to each plate. Cells will be pipetted off the plate and the cell suspension will be collected into a 50 ml conical centrifuge tube. Cells will then be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) will be prepared and maintained in accordance with the manufacturer's instructions. Assay Buffer should be prepared fresh for screening and contained 50 μl of Stimulation Buffer, 3 μl of test compound (12 μM final assay concentration) and 50 μl cells, Assay Buffer can be stored on ice until utilized. The assay can be initiated by addition of 50 μl of cAMP standards to appropriate wells followed by addition of 50 μl of PBS to wells H-11 and H12. Fifty μl of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. 100 μl of Detection Mix containing tracer cAMP will then be added to the wells. Plates were then incubated additional 2 hours followed by counting in a Wallac MicroBeta scintillation counter. Values of cAMP/well will then be extrapolated from a standard cAMP curve which is contained within each assay plate.

4. Reporter-Based Assays

a. Cre-Luc Reporter Assay (Gs-Associated Receptors)

293 and 293T cells are plated-out on 96 well plates at a density of 2×10⁴ cells per well and were transfected using Lipofectamine Reagent (BRL) the following day according to manufacturer instructions. A DNA/lipid mixture is prepared for each 6-well transfection as follows: 260 ng of plasmid DNA in 100 μl of DMEM is gently mixed with 41 of lipid in 100 μl of DMEM (the 260 ng of plasmid DNA consists of 200 ng of a 8xCRE-Luc reporter plasmid, 50 ng of pCMV comprising endogenous receptor or non-endogenous receptor or pCMV alone, and 10 ng of CMV-SEAP (secreted alkaline phosphatase expression plasmid; alkaline phosphatase activity is measured in the media of transfected cells to control for variations in transfection efficiency between samples). The 8XCRE-Luc reporter plasmid was prepared as follows: vector SRIF-β-gal was obtained by cloning the rat somatostatin promoter (−71/+51) at BglV-HindIII site in the pβgal-Basic Vector (Clontech). Eight (8) copies of cAMP response element were obtained by PCR from an adenovirus template AdpCF126CCRE8 [see, Suzuki et al., Hum Gene Ther (1996) 7:1883-1893; the disclosure of which is herein incorporated by reference in its entirety) and cloned into the SRIF-β-gal vector at the Kpn-BglV site, resulting in the 8xCRE-β-gal reporter vector. The 8xCRE-Luc reporter plasmid was generated by replacing the beta-galactosidase gene in the 8xCRE-β-gal reporter vector with the luciferase gene obtained from the pGL3-basic vector (Promega) at the HindIII-BamHI site. Following 30 min. incubation at room temperature, the DNA/lipid mixture is diluted with 400 μl of DMEM and 100 μl of the diluted mixture is added to each well. 100 μl of DMEM with 10% FCS are added to each well after a 4 hr incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

b. AP1 Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue # 219073) can be utilized following the protocol set forth above with respect to the CREB reporter assay, except that the components of the calcium phosphate precipitate were 410 ng pAP1-Luc, 80 ng pCMV-receptor expression plasmid, and 20 ng CMV-SEAP (secreted alkaline phosphatase expression plasmid; alkaline phosphatase activity is measured in the media of transfected cells to control for variations in transfection efficiency between samples).

c. Srf-Luc Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COST cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with, e.g. 1 μM, test compound. Cells are then lysed and assayed for luciferase activity using a Luclite™ Kit (Packard, Cat. # 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.).

d. Intracellular IP3 Accumulation Assay (Gq-Associated Receptors)

On day 1, cells comprising the receptors (endogenous or non-endogenous) can be plated onto 24 well plates, usually 1×10⁵ cells/well (although his number can be optimized. On day 2 cells can be transfected by first mixing 0.25 μg DNA in 50 μl serum free DMEM/well and 2 μl lipofectamine in 50 p. 1 serum free DMEM/well. The solutions are gently mixed and incubated for 15-30 min at room temperature. Cells are washed with 0.5 ml PBS and 400 μl of serum free media is mixed with the transfection media and added to the cells. The cells are then incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and optionally 50 μl of test compound to final concentration of 10 μM. The cells are then incubated for 30 min at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

### Example 4

### Fusion Protein Preparation

a. GPCR:Gs Fusion Constuct

The design of the GPCR-G protein fusion construct can be accomplished as follows: both the 5′ and 3′ ends of the rat G protein Gsα (long form; Itoh, H. et al., 83 PNAS 3776 (1986)) are engineered to include a HindIII (5′-AAGCTT-3′) sequence thereon. Following confirmation of the correct sequence (including the flanking HindIII sequences), the entire sequence is shuttled into pcDNA3.1(−) (Invitrogen, cat. no. V795-20) by subcloning using the HindIII restriction site of that vector. The correct orientation for the Gsα sequence is determined after subcloning into pcDNA3.1(−). The modified pcDNA3.1(−) containing the rat Gsα gene at HindIII sequence is then verified; this vector is now available as a “universal” Gsα protein vector. The pcDNA3.1(−) vector contains a variety of well-known restriction sites upstream of the HindIII site, thus beneficially providing the ability to insert, upstream of the Gs protein, the coding sequence of, e.g., an endogenous, constitutively active GPCR. This same approach can be utilized to create other “universal” G protein vectors, and, of course, other commercially available or proprietary vectors known to the artisan can be utilized—the important criteria is that the sequence for the GPCR be upstream and in-frame with that of the G protein.

a. Gq(6 Amino Acid Deletion)/Gi Fusion Construct

A Gq(del)/Gi fusion construct is a chimeric G protein whereby the first six (6) amino acids of the Gq-protein α-subunit (“Gαq”) are deleted and the last five (5) amino acids at the C-terminal end of Gαq are replaced with the corresponding amino acids of the Gαi subunit. A Gq(del)/Gi fusion construct will force an endogenous (for example) Gi coupled receptor to couple to its non-endogenous G protein, Gq (in the form of Gq(del)/Gi), such that the second messenger, for example, inositol triphosphate or diacylglycerol or Ca²⁺, can be measured in lieu of cAMP production.

### Example 5

### [³⁵S] GTPγS Assay

1. Membrane Preparation

In some embodiments membranes comprising a Target GPCR and for use in the identification of candidate compounds as, e.g., inverse agonists, agonists, or antagonists, are preferably prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4.

b. Procedure

All materials will be kept on ice throughout the procedure. Firstly, the media will be aspirated from a confluent monolayer of cells, followed by rinse with 10 ml cold PBS, followed by aspiration. Thereafter, 5 ml of Membrane Scrape Buffer will be added to scrape cells; this will be followed by transfer of cellular extract into 50 ml centrifuge tubes (centrifuged at 20,000 rpm for 17 minutes at 4° C.). Thereafter, the supernatant will be aspirated and the pellet will be resuspended in 30 ml Membrane Wash Buffer followed by centrifuge at 20,000 rpm for 17 minutes at 4° C. The supernatant will then be aspirated and the pellet resuspended in Binding Buffer. This will then be homogenized using a Brinkman Polytron™ homogenizer (15-20 second bursts until the all material is in suspension). This is referred to herein as “Membrane Protein”.

2. Bradford Protein Assay

Following the homogenization, protein concentration of the membranes will be determined using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a Polytron at about 12×1,000 rpm for about 5-10 seconds; it is noted that for multiple preparations, the homogenizer should be thoroughly cleaned between homogenization of different preparations).

a. Materials

Binding Buffer (as per above); Bradford Dye Reagent; Bradford Protein Standard will be utilized, following manufacturer instructions (Biorad, cat. no. 500-0006).

b. Procedure

Duplicate tubes will be prepared, one including the membrane, and one as a control “blank”. Each contained 800 μl Binding Buffer. Thereafter, 10 μl of Bradford Protein Standard (1 mg/ml) will be added to each tube, and 10 μl of membrane Protein will then be added to just one tube (not the blank). Thereafter, 200 μl of Bradford Dye Reagent will be added to each tube, followed by vortex of each. After five (5) minutes, the tubes will be re-vortexed and the material therein will be transferred to cuvettes. The cuvettes will then be read using a CECIL 3041 spectrophotometer, at wavelength 595 nm.

3. Identification Assay

a. Materials

GDP Buffer consists of 37.5 ml Binding Buffer and 2 mg GDP (Sigma, cat. no. G-7127), followed by a series of dilutions in Binding Buffer to obtain 0.2 μM GDP (final concentration of GDP in each well was 0.1 μM GDP); each well comprising a candidate compound, has a final volume of 200 μl consisting of 100 μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds will be preferably screened using a 96-well plate format (these can be frozen at −80° C.). Membrane Protein (or membranes with expression vector excluding the Target GPCR, as control), will be homogenized briefly until in suspension. Protein concentration will then be determined using the Bradford Protein Assay set forth above. Membrane Protein (and control) will then be diluted to 0.25 mg/ml in Binding Buffer (final assay concentration, 12.5 μg/well). Thereafter, 100 μl GDP Buffer is added to each well of a Wallac Scintistrip™ (Wallac). A 5 ul pin-tool will then be used to transfer 5 μA of a candidate compound into such well (i.e., 50 in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×)—excess liquid should be shaken from the tool after each rinse and dried with paper and kimwipes. Thereafter, 50 μl A of Membrane Protein will be added to each well (a control well comprising membranes without the Target GPCR was also utilized), and pre-incubated for 5-10 minutes at room temperature. Thereafter, 50 μl of [³⁵S]GTPγS (0.6 nM) in Binding Buffer will be added to each well, followed by incubation on a shaker for 60 minutes at room temperature (again, in this example, plates were covered with foil). The assay will then be stopped by spinning of the plates at 4000 RPM for 15 minutes at 22° C. The plates will then be aspirated with an 8 channel manifold and sealed with plate covers. The plates will then be read on a Wallac 1450 using setting “Prot. #37” (as per manufacturer's instructions).

### Example 6

### Cyclic Amp Assay

Another assay approach for identifying candidate compounds as, e.g., inverse agonists, agonists, or antagonists, is accomplished by utilizing a cyclase-based assay. In addition to so identifying candidate compounds, this assay approach can be utilized as an independent approach to provide confirmation of the results from the [³⁵S]GTPγS approach as set forth in Example 5, supra.

A modified Flash Plate™ Adenylyl Cyclase kit (New England Nuclear; Cat. No. SMP004A) is preferably utilized for identification of candidate compounds as modulators of a Target GPCR in accordance with the following protocol.

Cells transfected with the Target GPCR are harvested approximately three days after transfection. Membranes are prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000×g for 15 minutes at 4° C. The resulting pellet is then resuspended in buffer containing 20 mM HEPES, pH 7.4 and 0.1 mM EDTA, homogenized for 10 seconds, followed by centrifugation at 49,000×g for 15 minutes at 4° C. The resulting pellet is then stored at −80° C. until utilized. On the day of direct identification screening, the membrane pellet is slowly thawed at room temperature, resuspended in buffer containing 20 mM HEPES, pH 7.4 and 10 mM MgCl₂, to yield a final protein concentration of 0.60 mg/ml (the resuspended membranes are placed on ice until use).

cAMP standards and Detection Buffer (comprising 2 μCi of tracer {[¹²⁵I]cAMP (100 μl) to 11 ml Detection Buffer] are prepared and maintained in accordance with the manufacturer's instructions. Assay Buffer is prepared fresh for screening and contains 20 mM HEPES, pH 7.4, mM MgCl₂, 20 mM phosphocreatine (Sigma), 0.1 units/ml creatine phosphokinase (Sigma), 50 μM GTP (Sigma), and 0.2 mM ATP (Sigma); Assay Buffer is then stored on ice until utilized.

Candidate compounds are added, preferably, to e.g. 96-well plate wells (3 μl/well; 12 μM final assay concentration), together with 40 μl Membrane Protein (30 μg/well) and 50 μl of Assay Buffer. This admixture was then incubated for 30 minutes at room temperature, with gentle shaking.

Following the incubation, 100 μl of Detection Buffer is added to each well, followed by incubation for 2-24 hours. Plates are then counted in a Wallac MicroBeta™ plate reader using “Prot. #31” (as per manufacturer's instructions).

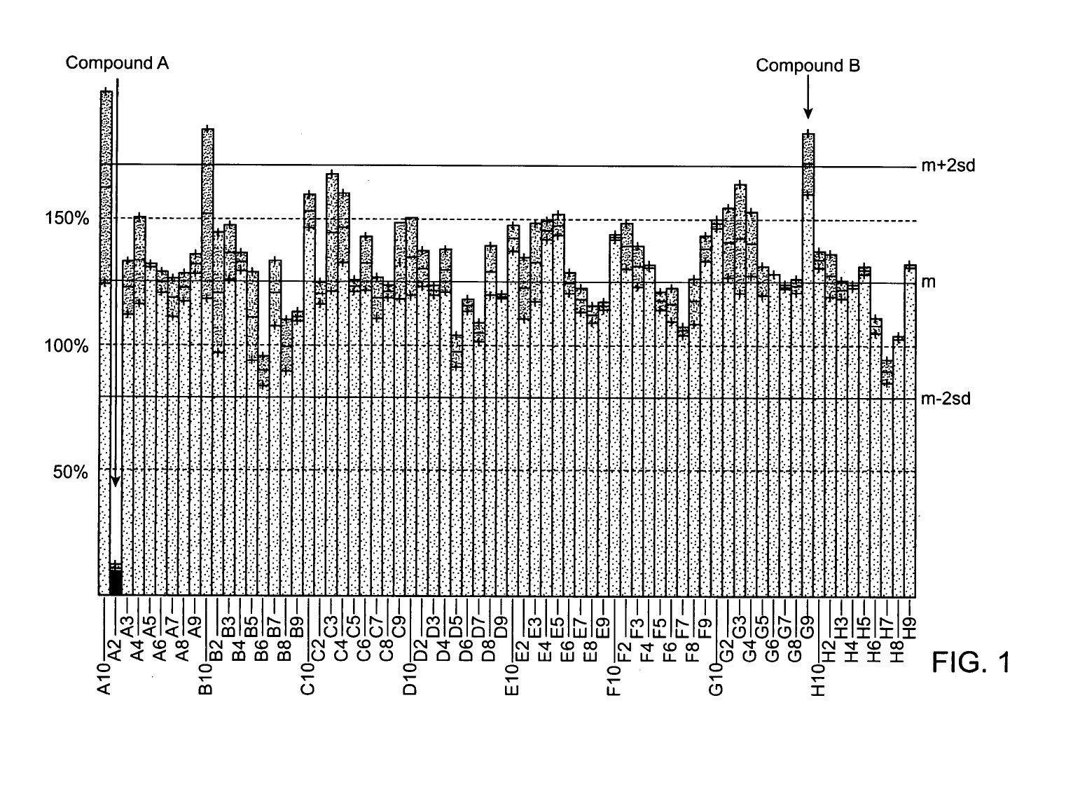

By way of example and not limitation, an illustrative screening assay plate (96 well format) result obtained is presented in FIG. 1. Each bar represents the result for a compound that differs in each well, the “Target GPCR” being a Gsα Fusion Protein construct of an endogenous, constitutively active Gs-coupled GPCR unrelated to BRS-3 receptor. The results presented in FIG. 1 also provide standard deviations based upon the mean results of each plate (“m”) and the mean plus two arbitrary preference for selection of inverse agonists as “leads” from the primary screen involves selection of candidate compounds that that reduce the percent response by at least the mean plate response, minus two standard deviations. Conversely, an arbitrary preference for selection of agonists as “leads” from the primary screen involves selection of candidate compounds that increase the percent response by at least the mean plate response, plus the two standard deviations. Based upon these selection processes, the candidate compounds in the following wells were directly identified as putative inverse agonist (Compound A) and agonist (Compound B) to said endogenous GPCR in wells A2 and G9, respectively. See, FIG. 1. It is noted for clarity: these compounds have been directly identified without any knowledge of the endogenous ligand for this GPCR. By focusing on assay techniques that are based upon receptor function, and not compound binding affinity, it is possible to ascertain compounds that are able to reduce the functional activity of this receptor (Compound A) as well as increase the functional activity of the receptor (Compound B).

### Example 7

### Fluorometric Imaging Plate Reader (FLIPR) Assay for the Measurement of Intracellular Calcium Concentration

Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F 14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO₂ is allowed to proceed for 60 min.

After the 1 hr incubation, the Fluo4-AM incubation buffer is removed and the cells are washed 2× with 100 μl wash buffer. In each well is left 100 μl wash buffer. The plate is returned to the incubator at 37° C./5% CO₂ for 60 min.

FLIPR (Fluorometric Imaging Plate Reader; Molecular Device) is programmed to add 50 μl candidate compound on the 30th second and to record transient changes in intracellular calcium concentration ([Ca²⁺]) evoked by the candidate compound for another 150 seconds. Total fluorescence change counts are used to determine agonist activity using the FLIPR software. The instrument software normalizes the fluorescent reading to give equivalent initial readings at zero.

By way of illustration and not limitation, the skilled artisan would appreciate that a candidate compound can be screened as an antagonist of the receptor by assessing its ability to inhibit the transient increase in intracellular ([Ca²⁺]) evoked by subsequent contact with a known agonist.

In some embodiments, the cells comprising Target Receptor further comprise Gα15, Gα16, or Gq(del)/Gi chimeric G protein.

Although the foregoing provides a FLIPR assay for agonist activity using stably transfected cells, a person of ordinary skill in the art would readily be able to modify the assay in order to characterize, e.g., antagonist activity. The person of ordinary skill in the art would also readily appreciate that, alternatively, transiently transfected cells could be used.

### Example 8

### MAP Kinase Assay

MAP kinase (mitogen activated kinase) may be monitored to evaluate receptor activation. MAP kinase can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the test compound and then extracted with Laemmli buffer. The soluble fraction is applied to an SDS-PAGE gel and proteins are transferred electrophoretically to nitrocellulose or Immobilin. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the test compound and a soluble extract is prepared. The extract is incubated at 30° C. for 10 min with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P is a liquid scintillation counter. Alternatively, the cell extract is incubated with gamma-³²P-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then be aspirated through the filter, which retains, the phosphorylated myelin basic protein. The filter is washed and counted for ³²P by liquid scintillation counting.

### Example 9

### Melanophore Technology

Melanophores are skin cells found in lower vertebrates. They contain pigmented organelles termed melanosomes. Melanophores are able to redistribute these melanosomes along a microtubule network upon G-protein coupled receptor (GPCR) activation. The result of this pigment movement is an apparent lightening or darkening of the cells. In melanophores, the decreased levels of intracellular cAMP that result from activation of a G1-coupled receptor cause melanosomes to migrate to the center of the cell, resulting in a dramatic lightening in color. If cAMP levels are then raised, following activation of a Gs-coupled receptor, the melanosomes are re-dispersed and the cells appear dark again. The increased levels of diacylglycerol that result from activation of Gq-coupled receptors can also induce this re-dispersion. In addition, the technology is also suited to the study of certain receptor tyrosine kinases. The response of the melanophores takes place within minutes of receptor activation and results in a simple, robust color change. The response can be easily detected using a conventional absorbance microplate reader or a modest video imaging system. Unlike other skin cells, the melanophores derive from the neural crest and appear to express a full complement of signaling proteins. In particular, the cells express an extremely wide range of G-proteins and so are able to functionally express almost all GPCRs.

Melanophores can be utilized to identify compounds, including natural ligands, against GPCRs. This method can be conducted by introducing test cells of a pigment cell line capable of dispersing or aggregating their pigment in response to a specific stimulus and expressing an exogenous clone coding for the GCPR. A stimulant, e.g., melatonin, sets an initial state of pigment disposition wherein the pigment is aggregated within the test cells if activation of the GPCR induces pigment dispersion. However, stimulating the cell with a stimulant to set an initial state of pigment disposition wherein the pigment is dispersed if activation of the GPCR induces pigment aggregation. The test cells are then contacted with chemical compounds, and it is determined whether the pigment disposition in the cells changed from the initial state of pigment disposition. Dispersion of pigments cells due to the candidate compound, including but not limited to a ligand, coupling to the GPCR will appear dark on a petri dish, while aggregation of pigments cells will appear light.

Materials and methods can be followed according to the disclosure of U.S. Pat. No. 5,462,856 and U.S. Pat. No. 6,051,386. These patent disclosures are herein incorporated by reference in their entirety.

The cells are plated in e.g. 96-well plates (one receptor per plate). 48 hours post-transfection, half of the cells on each plate are treated with 10 nM melatonin. Melatonin activates an endogenous G1-coupled receptor in the melanophores and causes them to aggregate their pigment. The remaining half of the cells are transferred to serum-free medium 0.7×L-15 (Gibco). After one hour, the cells in serum-free media remain in a pigment-dispersed state while the melatonin-treated cells are in a pigment-aggregated state. At this point, the cells are treated with a dose response of a test/candidate compound. If the plated GPCRs bind to the test/candidate compound, the melanophores would be expected to undergo a color change in response to the compound. If the receptor were either a Gs or Gq coupled receptor, then the melatonin-aggregated melanophores would undergo pigment dispersion. In contrast, if the receptor was a G1-coupled receptor, then the pigment-dispersed cells would be expected to undergo a dose-dependent pigment aggregation.

### Example 10

### BRS-3 Receptor Increases Intracellular IP3 Accumulation

COS-7 cells were transiently transfected with pCMV expression vector containing cDNA encoding endogenous human BRS-3 or with pCMV vector alone. Intracellular IP3 accumulation was read out as accumulation of total inositol phosphates.

COS-7 cells were plated at 10,000 cells per well in a 96-well plate and allowed to attach overnight. The COS-7 cells were then transfected in triplicate with 0.5 or 1.3 ng/well BRS-3/pCMV or with 13 ng/well empty pCMV, using Lipofectamine™ 2000 (Invitrogen #11668-027). After about 15 h, the transfected COS-7 cells were returned to complete medium (DMEM containing 10% FBS, 1% L-glutamine, and 1.5 g/L sodium bicarbonate) and cell culture was continued at 37° C. for about 8 hours.

The COS-7 cells were used in IP3 assay about 24 h post-transfection as described here. The complete medium was replaced with 100 μl inositol-free medium (Invitrogen/Gibco formula 02-5092EA; DMEM containing D-glucose, L-glutamine, phenol red, and pyridoxine HCl, and without inositol, sodium bicarbonate, and sodium pyruvate) supplemented with 1.5 g/L sodium bicarbonate and 4 μCi/ml [³H]myo-inositol (Perkin Elmer Life Sciences), and the cells were allowed to incubate for about 15 h at 37° C. The medium was then removed by aspiration and replaced with IP3 medium (inositol-free medium as above supplemented with 10 μM pargyline and 10 mM lithium chloride), and the cells were incubated for 3 hours at 37° C. (To screen a test compound as a BRS-3 agonist, the test compound would be included in this 3 h incubation.) Following incubation, the medium was removed by aspiration and replaced with buffer containing ice cold 0.1M formic acid. The plates were then frozen overnight at −80° C. to achieve complete cell lysis following an initial 30 min incubation on dry ice.

Following complete cell lysis, the assay plates were thawed in a 37° C. oven. The thawed contents were then transferred to 96-well filter plates (Millipore, Multiscreen) pre-loaded with resin (Biorad, AG1-X8 100-200 mesh, formate form). The plate was filtered using a vacuum manifold and the resin was washed multiple times with water. An elution buffer was then applied (200 μl, 1.0M ammonium formate/0.1M formic acid) and the resulting eluent was collected, under vacuum, in a 96-well collection plate. Aliquots of the eluent (2000) were transferred to scintillation vials containing 4 ml scintillation fluid and counted on a scintillation counter (Perkin Elmer Life Sciences, Optiphase Supermix or Hi-Safe 3).

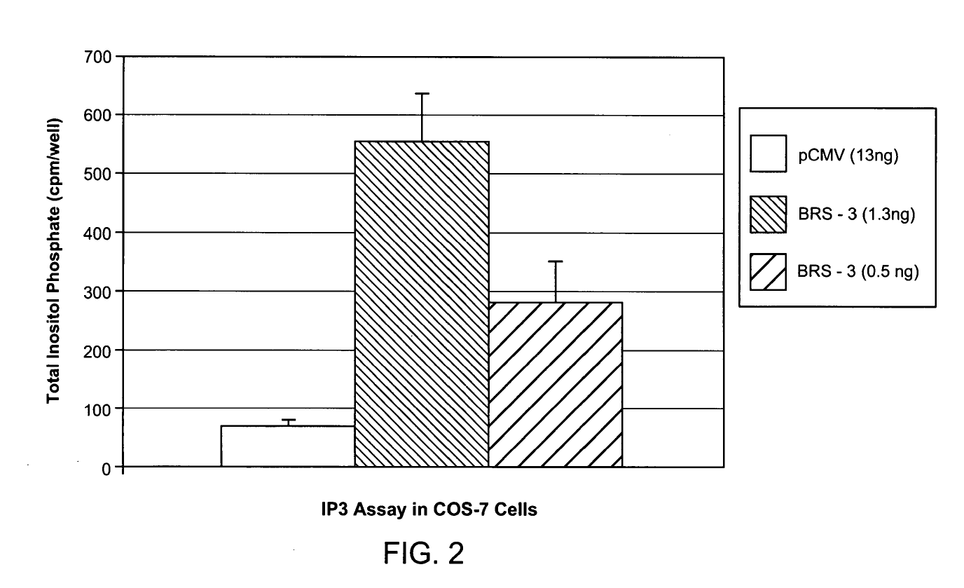

BRS-3 was found to exhibit a detectable level of constitutive activity and to increase IP3 accumulation in COS-7 cells. See FIG. 2. Analogous results were obtained using 293 cells (not shown).

### Example 11

### Agonist Activity of [D-Tyr⁶,βAla¹¹,Phe¹³,Nle¹⁴] Bombesin(6-14) at BRS-3

The agonist activity of [D-Tyr⁶,βAla¹¹,Phe¹³,Nle¹⁴]Bombesin(6-14) was characterized at BRS-3. [D-Tyr⁶,βAla¹¹,Phe¹³,Nle¹⁴]Bombesin(6-14) corresponds to Compound D34 in Table D.

A. FLIPR Assay

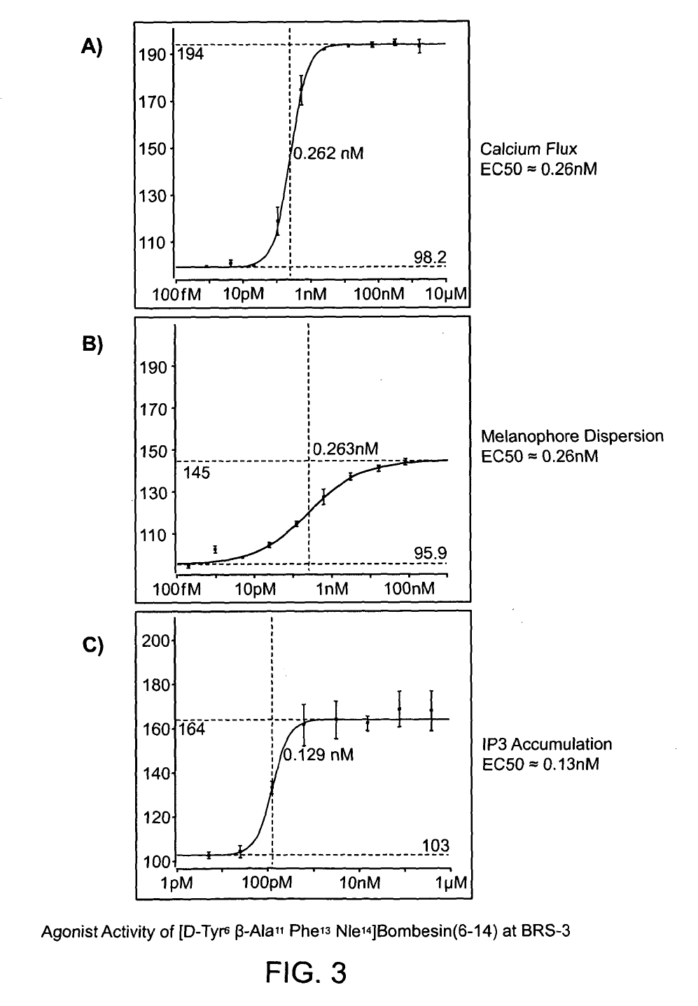

HeLa cells were stably transfected with plasmid DNA encoding human BRS-3 and used in fluorometric imaging plate reader (FLIPR) assay. In the representative assay shown in FIG. 3A, [D-Tyr⁶,βAla¹¹,Phe¹³,Nle¹⁴]Bombesin(6-14) was found to have an EC₅₀ at BRS-3 of about 0.26 nM in FLIPR assay.

B. Melanophore Assay

Melanophores were transiently transfected with plasmid DNA encoding human BRS-3 and used in dispersion assay. In the representative assay shown in FIG. 3B, [D-Tyr^{6,β3}Ala¹¹,Phe¹³,Nle¹⁴]Bombesin(6-14) was found to have an EC₅₀ at BRS-3 of about 0.26 nM in melanophore dispersion assay.

C. IP3 Assay

HeLa cells were stably transfected with plasmid DNA encoding human BRS-3 and used in IP3 accumulation assay. In the representative assay shown in FIG. 3C, [D-Tyr⁶,βAla¹¹,Phe¹³,Nle¹⁴]Bombesin(6-14) was found to have an EC₅₀ at BRS-3 of about 0.13 nM in T3 accumulation assay.

### Example 12

### Rat Polysomnography Protocol for Showing Promotion of Sleep by a Compound of the Invention

A compound of the invention that inhibits the activity of BRS-3 receptor, e.g. a compound having inverse agonist activity or antagonist activity at BRS-3, can be shown to be a compound suitable for promoting sleep (e.g., to be a sleep-promoting agent) using the exemplary, polysomnography protocol below.

Animals: Male Sprague-Dawley rats (225-350 g) (Harlan, San Diego, Calif.) are singly housed and maintained in a humidity—(30-70%) and temperature—(20-22° C.) controlled facility on a 12 h:12 h light/dark cycle (lights on at 6:30 A.M.) with free access to food (Harlan-Teldad Western Res., Orange, Calif., Rodent Diet 8604) and water. Rats are allowed at least three days of habituation to the animal facility before surgery.

Procedures:

Rats are anaesthetized with a ketamine/xylazine mixture, and surgically prepared for EEG (electroencephalogram) and electromyogram (EMG) recording. After 2-3 weeks of post-surgical recovery, rats are habituated to polypropylene test cages for at least three days. On test days, the rats are placed in the test chambers and habituated overnight. At 10 am the next day, the rats are administered the test compound (e.g., a compound that inhibits activity of BRS-3, such as an inverse agonist or antagonist of BRS-3), connected to the recording apparatus, and placed back into the test chambers for 3 h.

Data Analysis

EEG and EMG data are digitized and stored in 10 s epochs over the three hour test period. These data are then visually scored, and each 10 s epoch characterized as either a non-REM sleep, REM sleep, or waking episode. Total sleep time over the three hour period was calculated for each rat after either vehicle administration or test compound. Percent increase in sleep is then derived for each rat. A compound that increases the duration of sleep is a compound suitable for promoting sleep. A compound that promotes one or more of the reduction of the sleep onset latency period, reduction of the number of nighttime awakenings, and prolongation of the amount of time in delta-wave sleep without affecting REM sleep is a compound suitable for promoting sleep. A compound that promotes sleep consolidation is a compound suitable for promoting sleep.

It is expressly contemplated that the duration of the test period may be longer or shorter than 3 h and that the time of compound administration may be other than 10 am. It is also expressly contemplated that the polysomnography may be carried out in a mammal other than a rat. In certain embodiments, the mammal other than a rat is a non-human mammal. In certain embodiments, the mammal other than a rat is a mouse. In certain embodiments, the mammal other than a rat is a human.

### Example 13

### Rat Polysomnography Protocol for Showing Promotion of Wakefulness by a Compound of the Invention

A compound of the invention that stimulates the activity of BRS-3 receptor, e.g. a compound having agonist activity or partial agonist activity at BRS-3, can be shown to be a compound suitable for promoting wakefulness (e.g., to be a wakefulness-promoting agent) using the exemplary, polysomnography protocol below.

Animals: Male Sprague-Dawley rats (225-350 g) (Harlan, San Diego, Calif.) are singly housed and maintained in a humidity—(30-70%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Rats are allowed at least three days of habituation to the animal facility before surgery.

Procedures:

Rats are anaesthetized with a ketamine/xylazine mixture, and surgically prepared for EEG and EMG recording. After 2-3 weeks of post-surgical recovery, rats are habituated to polypropylene test cages for at least three days. On test days, the rats are placed in the test chambers and habituated overnight. At 10 am the next day, the rats are administered the test compound (e.g., a compound that stimulates activity of BRS-3, such as an agonist or partial agonist of BRS-3), connected to the recording apparatus, and placed back into the test chambers for 3 h.

Data Analysis

EEG (electroencephalogram) and EMG (electromyogram) data are digitized and stored in 10 s epochs over the three hour test period. These data were then visually scored, and each 10 s epoch characterized as either a non-REM sleep, REM sleep, or waking episode. Total wake time over the three hour period is calculated for each rat after either vehicle administration or test compound. Percent increase in wakefulness is then derived for each rat.

It is expressly contemplated that the duration of the test period may be longer or shorter than 3 h and that the time of compound administration may be other than 10 am. It is also expressly contemplated that the polysomnography may be carried out in a mammal other than a rat. In certain embodiments, the mammal other than a rat is a non-human mammal. In certain embodiments, the mammal other than a rat is a mouse. In certain embodiments, the mammal other than a rat is a human.

### Example 14

### Analysis of Co-Expression of BRS-3 and GAD67 Marker for GABAaergic Neurons in the Dorsomedial Hypothalamic Nucleus (DMH) and Other Subregions of Rat Hypothalamus

Co-expression of BRS-3 and GAD67 marker for GABAergic neurons in the dorsomedial hypothalamic nucleus (DMH) and other subregions of rat hypothalamus was investigated by in situ hybridization, using radiolabeled antisense probe for BRS-3 in combination with a digoxigenin (Dig)-labeled antisense probe for GAD67, a marker for GAGAergic neurons. Rat BRS-3 probe corresponding to a 450 base pair cDNA fragment corresponding to the 5′-terminal 450 nucleotides of the coding region of rat BRS-3 cDNA sequence (see, e.g., GenBank® Accession No. AF510984) was inserted into pBS vector (Stratagene, La Jolla, Calif.). Rat GAD67 probe corresponding to a 1300 base pair cDNA fragment derived from the coding region of rat GAD67 cDNA sequence (see, e.g., GenBank® Accession No. M76177) was inserted into pBS vector (Stratagene). In situ hybridization was carried out essentially as described below.

Rats were killed by rapid decapitation 1-2 h after initiation of the light cycle. Brains were removed, frozen in isopentane (−40° C.), and stored at −80° C. Serial 12-gm sections from the hypothalamus were prepared on a cryostat, thaw-mounted onto super frost plus slides, and stored at −80° C. until processing.

Sense and antisense ³³P radiolabeled probes were generated by in vitro transcription by incubating linearized plasmids in transcription buffer containing RNasin (40 units), DTT (2 mM), ATP, CTP and GTP (0.33 mM), [α-³³P]-UTP (Perkin Elmer, 50 μCi, NEG307 H001MC) and the appropriate polymerase (T7 50 units or T3 20 units). Probes were DNase treated, purified by ethanol precipitation and resuspend in 2× hybridization buffer (8× SET, 2×Denhardt's, 0.4% SDS, 200 mM dithiothreitol (DTT), 500 ug/ml tRNA, 50 ug/ml polyA, 50 ug/ml polyC).

Antisense digoxigenin labeled probes were generated by in vitro transcription by incubating linearized plasmids in transcription buffer containing RNasin (40 units), DTT (2 mM), nucleotide mix containing digoxigenin labeled UTP (rNTP digoxigenin RNA labeling mix, Roche #1277073) and the appropriate polymerase (17 50 units or T3 20 units). Probes were DNase treated and cleaned up through a centrisep column (Princeton Separations, # CS-901).

Tissue sections were removed from the freezer and allow to air dry for 15 min. Sections were subsequently fixed in 4% paraformaldehyde in phosphate buffer (0.1 M, pH 7.4) for 30 min at room temperature, rinsed 3 times in 1×PBS, and acetylated in 0.1M triethanolamine (TEA), pH 8.0 for 2 min then briefly in the same buffer containing 0.25% acetic anhydride. Slides were then rinsed for 5 minutes in 1×PBS and then dehydrated through graded alcohol concentrations and air dried. Radiolabeled probes were diluted in 2× hybridization buffer to yield an approximate concentration of 16×10⁶ cpm per slide. Salmon sperm was added at a final concentration of 20 ug/slide and digoxigenin labeled probe was added to a final concentration of 500 ng/slide. Dextran sulfate/Formamide (20%) was added to give a 1:1 ratio with 2× hybridization buffer. Diluted probe was placed on slides, coverslipped and were incubated at 55° C. for 16-18 hours in plastic trays humidified with 1×PBS. Coverslips were floated off with 1 mM DTT/4×SSC (600 mM sodium chloride and 60 mM sodium citrate, pH 7.2) and sections were subsequently washed once in 4×SSC for 10 min, incubated in ribonuclease A (200 ug/ml) for 60 min in a 37° C. water bath, then rinsed in 2×, 1×, and 0.5×SSC for 5 minutes each. Sections were washed to a final stringency of 0.1×SSC at 65° C. for 1 hour, then washed twice in 0.1×SSC then washed in TN (100 mM Tris, pH 7.5, 150 mM NaCl) for 5 minutes. Sections were then placed in 0.5% Casein/TN blocking solution for 30 minutes then incubated for 2 hours with anti-digoxigenin-AP antibody (Roche, #1093274) diluted 1:300 in 0.5% Casein/TN solution. Sections were then washed 3 times, 2 minutes each in TN and then 3 times 5 minutes each in TNM (100 mM Tris, pH 9.5, 100 mM NaCl, 50 mM MgCl₂). After the last wash, sections were incubated in color reaction (0.2 mg/ml levamisole, 3.4 ul/ml NBT (Roche #1383213), 3.5 ul/ml BCIP (Roche # 1383221) in TNM and 0.22 u sterile filtered) for 20-30 minutes and reaction stopped in TE for 30 minutes. Antibody was striped off by incubating sections in 0.1M glycine and 0.5% triton-X 100 for 10 minutes and washed in water. Sections were fixed in 2.5% glutaraldehyde for 1-2 hours and washed with water then air dried. Once section dried, they were exposed to x-ray sensitive film (Bio-Max, Kodak, Eastman Kodak Co., Rochester, N.Y.) for 2-7 days and dipped in photographic emulsion (Ilford Scientific K.5D Emulsion in gel form from Polysciences, #17537) dried and stored in slides box with desiccant at 4° C. for 4-8 weeks depending on the level of expression. After development of dipped slides following manufacturer recommendations (Kodak D19), sections were washed extensively in water and air dried then mounted with coverslips for microscopic examination.

Images of the distribution of BRS-3 and GAD67 mRNA-containing cells were obtained using an Olympus BX51 microscope connected to a videocamera (NTSC 750CE) using Stereoinvestigator® v6.55.2 software (Microbrightfield, VT). Nonradioactive riboprobes were visualized under brightfield as a purple precipitate, and radioactive probes were visualized under darkfield by silver grain distribution. For each pair of probes, analysis was carried out independently on the left and right side of three different sections.

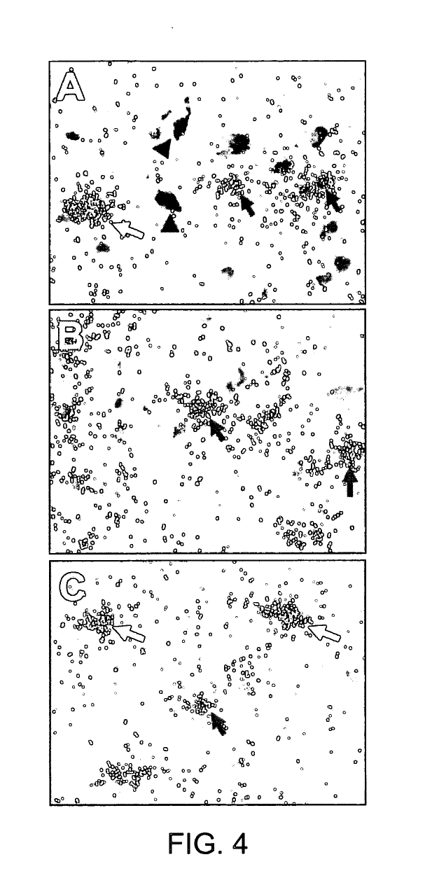

Representative photomicrographic images illustrating the expression of BRS-3 and GAD67 in the dorsomedial hypothalamic nucleus (DMH) in rat are presented as panels A to C in FIG. 4. Note the presence of neurons expressing only BRS-3 (white arrow), neurons expressing only GAD67 (solid arrowhead), and neurons co-expressing BRS-3 and GAD67 (black arrow).

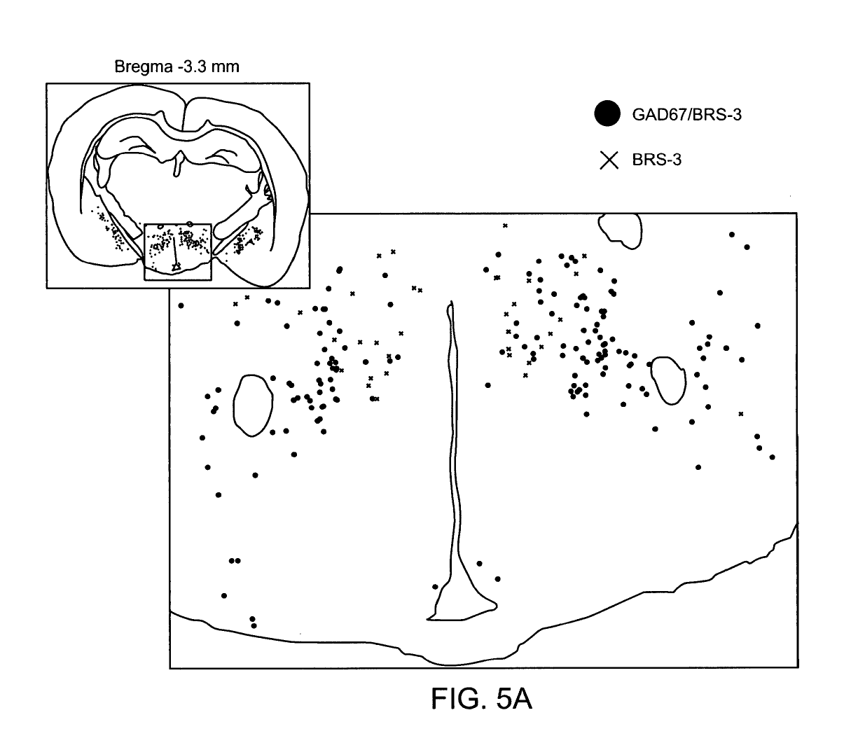

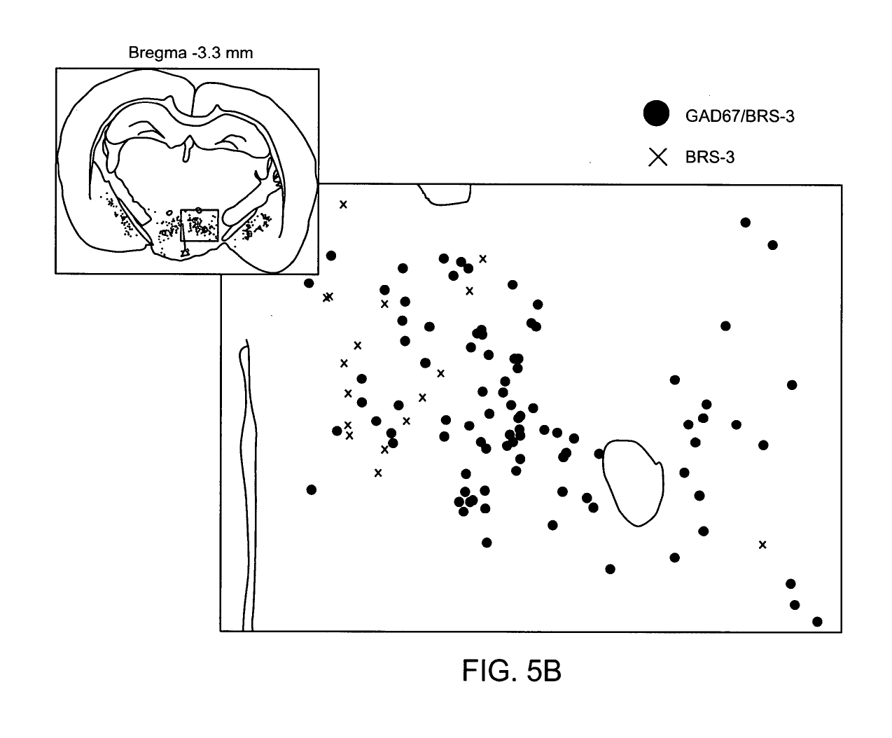

A representative graphic presentation of the expression of BRS-3 and GAD67 in the dorsomedial hypothalamic nucleus (DMH) in rat is shown in FIG. 5, where FIG. 5B is an enlargement of part of FIG. 5A. Neurons expressing only BRS-3 are shown as crosses (x), neurons co-expressing BRS-3 and GAD67 are shown as circles (•). Neurons expressing only GAD67 are not shown. The location of the tissue slice is indicated using coordinates relative to Bregma according to: The Rat Brain in Stereotaxic Coordinates, Paxinos and Watson, 4^{th} Edition, 1998, Academic Press, San Diego.

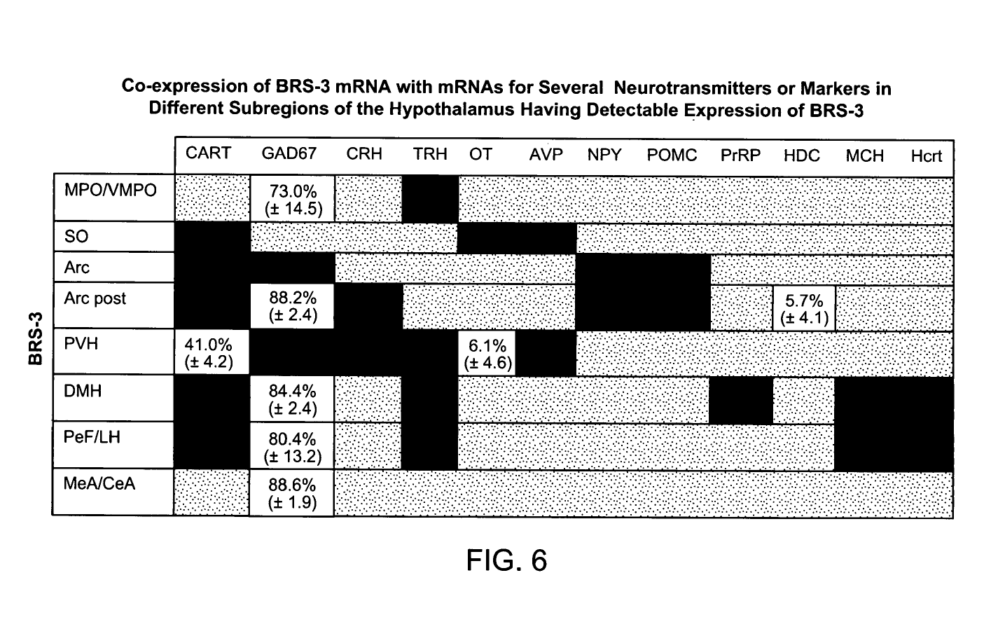

The results of in situ hybridization analysis of the co-expression of BRS-3 and a number of neurotransmitters or markers by neurons within subregions of the hypothalamus exhibiting detectable expression of BRS-3 is presented in FIG. 6. Results are shown for the following hypothalamic subregions: medial preoptic nucleus (MPO), ventromedial preoptic nucleus (VMPO), supraoptic nucleus (SO), arcuate nucleus (Arc), arcuate nucleus posterior part (Arc post), paraventricular nucleus of the hypothalamus (PVH), dorsomedial hypothalamic nucleus (DMH), lateral hypothalamic area (LH), medial amygdala (MeA), and central amygdala (CeA). Results are not presented for the ventrolateral preoptic nucleus (VLPO), as no expression of BRS-3 was detectable in this subregion. The following neurotransmitters or markers were interrogated for co-expression with BRS-3: cocaine-amphetamine regulated transcript (CART), glutamic acid decarboxylase 67 (GAD67), corticotrophin-releasing hormone (CRH), thyrotropin releasing hormone (TRH), oxytocin (OT), vasopressin (AVP), neuropeptide Y (NPY), proopiomelanocortin (POMC), prolactin-releasing peptide (PrRP), histidine decarboxylase (HDC), melanin-concentrating hormone (MCH), and hypocretin/orexin peptide (Hcrt). A black box indicates that no co-expression was found. A gray box indicates either that the neurotransmitter or marker is normally not found in this hypothalamic subregion or that the analysis was not carried out. In FIG. 6, results are presented for the hypothalamic subregion indicated as the percentage of BRS-3 expressing neurons therein having co-expression of the indicated neurotransmitter or marker. Surprisingly, marked co-expression of BRS-3 with GAD67, a marker for GABAergic neurons, was found (FIG. 6).

In the case of the dorsomedial hypothalamic nucleus (DMH), for example, about 84% of the BRS-3 expressing neurons therein were found to be GABAergic neurons (neurons expressing GAD67) (FIG. 6).

### Example 15

### Yeast Reporter Assay for BRS-3 Modulator (e.g., Inverse Agonist, Antagonist, Agonist, or Partial Agonist) Activity

The yeast cell-based reporter assays have previously been described in the literature (e.g., see Miret et al, J Biol Chem (2002) 277:6881-6887; Campbell et al, Bioorg Med Chem Lett (1999) 9:2413-2418; King et al, Science (1990) 250:121-123; WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast alpha-cell GPCR, Step 3 has been deleted to allow for a homologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing (3-galactosidase (LacZ) under the control of the Fus1 promoter (Fuslp), a system has been developed whereby receptor activation leads to an enzymatic readout.

Yeast cells are transformed by an adaptation of the lithium acetate method described by Agatep et al (Agatep et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells are grown overnight on yeast tryptone plates (YT). Carrier single-stranded DNA (10 μg), 2 μg of each of two Fuslp-LacZ reporter plasmids (one with URA selection marker and one with TRP), 2 μg of BRS-3 (e.g., human receptor) in yeast expression vector (2 μg origin of replication) and a lithium acetate/polyethylene glycol/TE buffer is pipetted into an Eppendorf tube. The yeast expression plasmid containing the receptor/no receptor control has a LEU marker. Yeast cells are inoculated into this mixture and the reaction proceeds at 30° C. for 60 min. The yeast cells are then heat-shocked at 42° C. for 15 min. The cells are then washed and spread on selection plates. The selection plates are synthetic defined yeast media minus LEU, URA and TRP (SD-LUT). After incubating at 30° C. for 2-3 days, colonies that grow on the selection plates are then tested in the LacZ assay.

In order to perform fluorimetric enzyme assays for β-galactosidase, yeast cells carrying the subject BRS-3 receptor are grown overnight in liquid SD-LUT medium to an unsaturated concentration (i.e. the cells are still dividing and have not yet reached stationary phase). They are diluted in fresh medium to an optimal assay concentration and 90 μl of yeast cells are added to 96-well black polystyrene plates (Costar). Test compounds, dissolved in DMSO and diluted in a 10% DMSO solution to 10× concentration, are added to the plates and the plates placed at 30° C. for 4 h. After 4 h, the substrate for the β-galactosidase is added to each well. In these experiments, Fluorescein di (β-D-galactopyranoside) is used (FDG), a substrate for the enzyme that releases fluorescein, allowing a fluorimetric read-out. 20 μl per well of 500 μM FDG/2.5% Triton X100 is added (the detergent is necessary to render the cells permeable). After incubation of the cells with the substrate for 60 min, 20 μl per well of 1M sodium carbonate is added to terminate the reaction and enhance the fluorescent signal. The plates are then read in a fluorimeter at 485/535 nm.

A decrease in fluorescent signal in BRS-3-transformed yeast cells over that in yeast cells transformed with empty vector is indicative of a test compound being a compound that inhibits BRS-3 receptor functionality (e.g., a compound that is an inverse agonist or antagonist of BRS-3). In certain embodiments, compounds of the invention give a decrease in fluorescent signal below that of the background signal (the signal obtained in the presence of vehicle alone).

An increase in fluorescent signal in BRS-3-transformed yeast cells over that in yeast cells transformed with empty vector is indicative of a test compound being a compound that stimulates BRS-3 receptor functionality (e.g., a compound that is an agonist or partial agonist of BRS-3). In certain embodiments, compounds of the invention give an increase in fluorescent signal above that of the background signal (the signal obtained in the presence of vehicle alone).

### Example 16

### Receptor Binding Assay

A test compound can be evaluated for its ability to reduce formation of the complex between a compound known to be a ligand of a G protein-coupled receptor of the invention and the receptor. In certain embodiments, the known ligand is radiolabeled. The radiolabeled known ligand can be used in a screening assay to identify/evaluate compounds. In general terms, a newly synthesized or identified compound (i.e., test compound) can be evaluated for its ability to reduce binding of the radiolabeled known ligand to the receptor, by its ability to reduce formation of the complex between the radiolabeled known ligand and the receptor.

In other aspect, a test compound can be radiolabeled and shown to be a ligand of a subject GPCR of the invention by evaluating its ability to bind to a cell comprising the subject GPCR or to membrane comprising the subject GPCR.

A level of specific binding of the radiolabled known ligand in the presence of the test compound less than a level of specific binding of the radiolabeled known ligand in the absence of the test compound is indicative of less of the complex between said radiolabeled known ligand and said receptor being formed in the presence of the test compound than in the absence of the test compound.

Assay Protocol for Detecting the Complex Between a Compound Known to be a Ligand of a G Protein-Coupled Receptor of the Invention and the Receptor

A. Preparation of the Receptor

293 cells are transiently transfected with 10 ug expression vector comprising a polynucleotide encoding a G protein-coupled receptor of the invention using 60 ul Lipofectamine (per 15-cm dish). The transiently transfected cells are grown in the dish for 24 hours (75% confluency) with a media change and removed with 10 ml/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 ul of appropriately diluted membranes (diluted in assay buffer containing 50 mM Tris HCl (pH 7.4), 10 mM MgCl₂, and 1 mM EDTA; 5-50 ug protein) is added to 96-well polypropylene microtiter plates followed by addition of 100 ul of assay buffer and 50 ul of a radiolabeled known ligand. For nonspecific binding, 50 ul of assay buffer is added instead of 100 ul and an additional 50 ul of 10 uM said known ligand which is not radiolabeled is added before 50 ul of said radiolabeled known ligand is added. Plates are then incubated at room temperature for 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plate are sealed, 50 ul of Optiphase Supermix is added to each well, the top of the plates are sealed, and plates are counted in a Trilux MicroBeta scintillation counter. For determining whether less of the complex between said radiolabeled known ligand and said receptor is formed in the presence of a test compound, instead of adding 100 ul of assay buffer, 100 ul of appropriately diluted said test compound is added to appropriate wells followed by addition of 50 ul of said radiolabled known ligand.

### Example 17

### Protection from Pentylenetetrazole-Induced Seizures

A compound of the invention can be shown to be a compound having anticonvulsant activity (e.g., to be an anticonvulsant agent) using the exemplary protocols below. A compound of the invention that inhibits the activity of BRS-3 receptor, e.g. a compound having inverse agonist activity or antagonist activity at BRS-3, can be shown to be a compound having anticonvulsant activity (e.g., to be an anticonvulsant agent) using the exemplary protocols below. A compound of the invention can be shown to be a compound having anti-migraine activity (e.g., to be an anti-migraine agent) using the exemplary protocols below. A compound of the invention that inhibits the activity of BRS-3 receptor, e.g. a compound having inverse agonist activity or antagonist activity at BRS-3, can be shown to be a compound having anti-migraine activity (e.g., to be an anti-migraine agent) using the exemplary protocols below. It is expressly contemplated that a drug (e.g., test compound or diazepam) may be administered, e.g., orally, intraperitoneally, intracerebroventricularly or intravenously. It is expressly contemplated that a protocol below may be carried out using a mammal other than a mouse. In certain embodiments, the mammal other than a mouse is a rat.

### Protocol I

Animals: Male B6.129X1-Brs3^{tm1Jfb}/J a mice (BRS-3 knockout mice; stock number 004366) weighing 20-22 g at the start of the experiment are purchased from Jackson Laboratory (Bar Harbor, Me.), along with control male C57BL/6J mice (stock number 000664). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

Pentylenetetrazole (Sigma Chemical Co.) is administered at 125 mg/kg subcutaneously to mice in experimental (BRS-3 knockout mouse alone and BRS-3 knockout mouse plus diazepam) and control (control mouse alone and control mouse plus diazepam) groups. The number of mice surviving is recorded for each group at 30 min and 60 min after administration of pentylenetetrazole.

Drug administration: Diazepam is administered 60 min before administration of pentylenetetrazole. Diazepam is administered at, e.g., 0.1 mg/kg, 0.3 mg/kg, 1 mg/kg, 3 mg/kg, 10 mg/kg, 30 mg/kg or 100 mg/kg.

Data Analysis

The data are presented for each group as the percentage of mice protected from death. A greater percentage of mice protected from death in the BRS-3 knockout mouse alone group in comparison with the percentage of mice protected from death in the control mouse alone group is indicative of a compound that inhibits the activity of BRS-3 receptor, e.g. a compound having inverse agonist activity or antagonist activity at BRS-3, being a compound having anticonvulsant activity (e.g., being an anticonvulsant agent). A greater percentage of mice protected from death in the BRS-3 knockout mouse plus diazepam group in comparison with the percentage of mice protected from death in the control mouse plus diazepam group is indicative of a compound that inhibits the activity of BRS-3 receptor, e.g. a compound having inverse agonist activity or antagonist activity at BRS-3, being a compound having anticonvulsant activity (e.g., being an anticonvulsant agent). A greater percentage of mice protected from death in the BRS-3 knockout mouse alone group in comparison with the percentage of mice protected from death in the control mouse alone group is indicative of a compound that inhibits the activity of BRS-3 receptor, e.g. a compound having inverse agonist activity or antagonist activity at BRS-3, being a compound having anti-migraine activity (e.g., being an anti-migraine agent). A greater percentage of mice protected from death in the BRS-3 knockout mouse plus diazepam group in comparison with the percentage of mice protected from death in the control mouse plus diazepam group is indicative of a compound that inhibits the activity of BRS-3 receptor, e.g. a compound having inverse agonist activity or antagonist activity at BRS-3, being a compound having anti-migraine activity (e.g., being an anti-migraine agent).

### Protocol II

Animals: Male CF1 mice weighing 20-22 g at the start of the experiment are purchased from Charles River Laboratories (Wilmington, Mass.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

Pentylenetetrazole (Sigma Chemical Co.) is administered at 125 mg/kg subcutaneously to mice in experimental (test compound alone and test compound plus diazepam) and control (vehicle-control and diazepam-control) groups. The number of mice surviving is recorded for each group at 30 min and 60 min after administration of pentylenetetrazole.

Drug administration: All drugs and vehicle are administered 60 min before administration of pentylenetetrazole. Drugs (e.g., test compounds and diazepam) are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that inhibits the activity of BRS-3 receptor, e.g. a compound having inverse agonist activity or antagonist activity at BRS-3.

Data Analysis

The data are presented for each group as the percentage of mice protected from death. A greater percentage of mice protected from death in a test compound alone experimental group in comparison with the percentage of mice protected from death in the vehicle-control group is indicative of the compound administered in the experimental group being a compound having anticonvulsant activity (e.g., being an anticonvulsant agent). A greater percentage of mice protected from death in a test compound plus diazepam experimental group in comparison with the percentage of mice protected from death in the diazepam-control group is indicative of the compound administered in the experimental group being a compound having anticonvulsant activity (e.g., being an anticonvulsant agent). A greater percentage of mice protected from death in a test compound alone experimental group in comparison with the percentage of mice protected from death in the vehicle-control group is indicative of the compound administered in the experimental group being a compound having anti-migraine activity (e.g., being an anti-migraine agent). A greater percentage of mice protected from death in a test compound plus diazepam experimental group in comparison with the percentage of mice protected from death in the diazepam-control group is indicative of the compound administered in the experimental group being a compound having anti-migraine activity (e.g., being an anti-migraine agent). It is expressly contemplated that said test compound which is a compound that inhibits the activity of BRS-3 receptor, e.g. a compound having inverse agonist activity or antagonist activity at BRS-3, can be shown to be a compound having anticonvulsant activity (e.g., to be an anticonvulsant agent). It is expressly contemplated that said test compound which is a compound that inhibits the activity of BRS-3 receptor, e.g. a compound having inverse agonist activity or antagonist activity at BRS-3, can be shown to be a compound having anti-migraine activity (e.g., to be an anti-migraine agent).

### Example 18

### Protection from Electroshock-Induced Seizures

A compound of the invention can be shown to be a compound having anticonvulsant activity (e.g., to be an anticonvulsant agent) using the exemplary protocols below. A compound of the invention that inhibits the activity of BRS-3 receptor, e.g. a compound having inverse agonist activity or antagonist activity at BRS-3, can be shown to be a compound having anticonvulsant activity (e.g., to be an anticonvulsant agent) using the exemplary protocols below. A compound of the invention can be shown to be a compound having anti-migraine activity (e.g., to be an anti-migraine agent) using the exemplary protocols below. A compound of the invention that inhibits the activity of BRS-3 receptor, e.g. a compound having inverse agonist activity or antagonist activity at BRS-3, can be shown to be a compound having anti-migraine activity (e.g., to be an anti-migraine agent) using the exemplary protocols below. It is expressly contemplated that a drug (e.g., test compound or diazepam) may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat.

### Protocol I

Animals: Male B6.129X1-Brs3^{tm1Jfb}/J mice (BRS-3 knockout mice; stock number 004366) weighing 20-22 g at the start of the experiment are purchased from Jackson Laboratory (Bar Harbor, Me.), along with control male C57BL/6J mice (stock number 000664). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

Electroshock is administered using a Ugo Basile ECT, Unit 7801 seizure apparatus (Ugo Basile, Italy) and corneal electodes soaked in 0.9% saline, to mice in experimental (BRS-3 knockout mouse alone and BRS-3 knockout mouse plus diazepam) and control (control mouse alone and control mouse plus diazepam) groups. Mice receive a shock of 30 mA for 0.3 sec.

Drug administration: Diazepam is administered 60 min before administration of electroshock. Diazepam is administered at, e.g., 0.1 mg/kg, 0.3 mg/kg, 1 mg/kg, 3 mg/kg, 10 mg/kg, 30 mg/kg or 100 mg/kg.

Data analysis

The data are presented for each group as the percentage of mice protected from the hind-limb extensor component of the seizure. A greater percentage of mice protected from said hind-limb extensor component in the BRS-3 knockout mouse alone group in comparison with the percentage of mice protected from said hind-limb extensor component in the control mouse alone group is indicative of a compound that inhibits the activity of BRS-3 receptor, e.g. a compound having inverse agonist activity or antagonist activity at BRS-3, being a compound having anticonvulsant activity (e.g., being an anticonvulsant agent). A greater percentage of mice protected from said hind-limb extensor component in the BRS-3 knockout mouse plus diazepam group in comparison with the percentage of mice protected from said hind-limb extensor component in the control mouse plus diazepam group is indicative of a compound that inhibits the activity of BRS-3 receptor, e.g. a compound having inverse agonist activity or antagonist activity at BRS-3, being a compound having anticonvulsant activity (e.g., being an anticonvulsant agent). A greater percentage of mice protected from said hind-limb extensor component in the BRS-3 knockout mouse alone group in comparison with the percentage of mice protected from said hind-limb extensor component in the control mouse alone group is indicative of a compound that inhibits the activity of BRS-3 receptor, e.g. a compound having inverse agonist activity or antagonist activity at BRS-3, being a compound having anti-migraine activity (e.g., being an anti-migraine agent). A greater percentage of mice protected from said hind-limb extensor component in the BRS-3 knockout mouse plus diazepam group in comparison with the percentage of mice protected from said hind-limb extensor component in the control mouse plus diazepam group is indicative of a compound that inhibits the activity of BRS-3 receptor, e.g. a compound having inverse agonist activity or antagonist activity at BRS-3, being a compound having anti-migraine activity (e.g., being an anti-migraine agent).

### Protocol II

Animals: Male CF1 mice weighing 20-22 g at the start of the experiment are purchased from Charles River Laboratories (Wilmington, Mass.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teldad Western Res., Orange, Calif., Rodent Diet 8604) and water. Mice are allowed at least three days of habituation to the animal facility before testing.

Procedures:

Electroshock is administered using a Ugo Basile ECT, Unit 7801 seizure apparatus (Ugo Basile, Italy) and corneal electodes soaked in 0.9% saline, to mice in experimental (test compound alone and test compound plus diazepam) and control (vehicle-control and diazepam-control) groups. Mice receive a shock of 30 mA for 0.3 sec.

Drug administration: All drugs and vehicle are administered 60 min before administration of electroshock. Drugs (e.g., test compounds and diazepam) are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that inhibits the activity of BRS-3 receptor, e.g. a compound having inverse agonist activity or antagonist activity at BRS-3.

Data Analysis

The data are presented for each group as the percentage of mice protected from the hind-limb extensor component of the seizure. A greater percentage of mice protected from said hind-limb extensor component in a test compound alone group in comparison with the percentage of mice protected from said hind-limb extensor component in the vehicle-control group is indicative of the compound administered in the experimental group being a compound having anticonvulsant activity (e.g., being an anticonvulsant agent). A greater percentage of mice protected from said hind-limb extensor component in a test compound plus diazepam group in comparison with the percentage of mice protected from said hind-limb extensor component in the diazepam-control group is indicative of the compound administered in the experimental group being a compound having anticonvulsant activity (e.g., being an anticonvulsant agent). A greater percentage of mice protected from said hind-limb extensor component in a test compound alone group in comparison with the percentage of mice protected from said hind-limb extensor component in the vehicle-control group is indicative of the compound administered in the experimental group being a compound having anti-migraine activity (e.g., being an anti-migraine agent). A greater percentage of mice protected from said hind-limb extensor component in a test compound plus diazepam group in comparison with the percentage of mice protected from said hind-limb extensor component in the diazepam-control group is indicative of the compound administered in the experimental group being a compound having anti-migraine activity (e.g., being an anti-migraine agent). It is expressly contemplated that said test compound which is a compound that inhibits the activity of BRS-3 receptor, e.g. a compound having inverse agonist activity or antagonist activity at BRS-3, can be shown to be a compound having anticonvulsant activity (e.g., to be an anticonvulsant agent). It is expressly contemplated that said test compound which is a compound that inhibits the activity of BRS-3 receptor, e.g. a compound having inverse agonist activity or antagonist activity at BRS-3, can be shown to be a compound having anti-migraine activity (e.g., to be an anti-migraine agent).

### Example 19

### Porsolt Forced Swim Test

The effects measured in this model have been correlated to antidepressant activity for drugs. The paradigm of this model is that an effective antidepressant compound will cause a mouse or rat to make greater attempts to escape a water-filled cylinder than a mouse or rat given vehicle only.

A compound of the invention can be shown to be a compound having antidepressant activity (e.g., to be an antidepressant agent) using the exemplary protocols below. A compound of the invention that inhibits the activity of BRS-3 receptor, e.g. a compound having inverse agonist activity or antagonist activity at BRS-3, can be shown to be a compound having antidepressant activity (e.g., to be an antidepressant agent) using the exemplary protocols below. It is expressly contemplated that a test compound may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat. It is expressly contemplated that a protocol below may alternatively be carried out using a mammal other than a rat. In certain embodiments, the mammal other than a rat is a mouse.

### Protocol I

Animals: Animals used in this study are non-na\ddot{v}e male B6.129X1-Brs3^{tm1Jfb}/J mice (BRS-3 knockout mice; stock number 004366) and control non-naïve male C57BL/6J mice (stock number 000664) weighing 20-22 g at the start of the experiment (Jackson Laboratory, Bar Harbor, Me.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

The test apparatus consists of 6 clear Plexiglas cylinders 20 cm high×10 cm wide. Cylinders are filled to 9 cm with 25° C. water. Each mouse is placed in a cylinder for a 5-10 minute training session. Mice are brought back 24 h later for a 5 min test session. The test session is videotaped for later scoring.

Data Analysis

Scoring is done for each group using a time-sampling computer program written in Visual Basic and run in DOS. Every five seconds, mice are rated as demonstrating one of three behaviors: immobility, mild swim, or climbing. These sampling scores are then converted into percentages of the test session. Greater attempts to escape a water-filled cylinder made by mice in the BRS-3 knockout mouse group in comparison with attempts to escape a water-filled cylinder made by mice in the control mouse group is indicative of a compound that inhibits the activity of BRS-3 receptor, e.g. a compound having inverse agonist activity or antagonist activity at BRS-3, being a compound having antidepressant activity (e.g., being an antidepressant agent).

### Protocol II

Animals: Animals used in this study are non-naïve male Sprague Dawley rats (SASCO, St Louis) weighing between 280-350 g. The rats are housed two per cage and maintained in a humidity—(40-60%) and temperature—(20-22° C.) controlled facility on a 12 h:12 h light/dark cycle (lights on at 6:30 A.M.) with free access to food (Harlan-Teldad Western Res., Orange, Calif., Rodent Diet 8604) and water. Rats are allowed at least three days of habituation to the animal facility before testing.

Procedures:

The test apparatus consists of 6 clear Plexiglas cylinders 40 cm high×19 cm wide. Cylinders are filled to 18 cm with 25° C. water. Each rat is placed in a cylinder for a 15 minute training session. Following either subchronic or acute dosing of either vehicle or test compound, rats—in experimental (test compound) and control (vehicle) groups—are brought back 24 h later for a 5 min test session. The test session is videotaped for later scoring.

Drug administration: Subchronic dosing consists of administering drug three times in the 24-hour period between training and testing. The drug is administered 24 h, 5 h and 1 h prior to the test session. Acute dosing consists of administering the drug once, 1 hour prior to the test session. Test compounds are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that inhibits the activity of BRS-3 receptor, e.g. a compound having inverse agonist activity or antagonist activity at BRS-3.

Data Analysis

Scoring is done for each group using a time-sampling computer program written in Visual Basic and run in DOS. Every five seconds, rats are rated as demonstrating one of three behaviors: immobility, mild swim, or climbing. These sampling scores are then converted into percentages of the test session. Greater attempts to escape a water-filled cylinder made by rats in a test compound group in comparison with attempts to escape a water-filled cylinder made by rats in the vehicle-control group is indicative of the compound administered in the experimental group being a compound having antidepressant activity (e.g., being an antidepressant agent). It is expressly contemplated that said test compound which is a compound that inhibits the activity of BRS-3 receptor, e.g. a compound having inverse agonist activity or antagonist activity at BRS-3, can be shown to be a compound having antidepressant activity (e.g., to be an antidepressant agent).

### Example 20

### Elevated Plus Maze

The elevated plus maze model capitalizes on mice and rats' innate fear of open, elevated spaces. The test apparatus is an elevated plus maze consisting of two open arms and two closed arms. A mouse or rat will naturally choose to spend more time on the closed arms of the maze than on the open arms, but if an efficacious anxiolytic compound is administered to the mouse or rat prior to the test the amount of time the mouse or rat spends in the open arms is increased.

A compound of the invention can be shown to be a compound having anxiolytic activity (e.g., to be an anxiolytic agent) using the exemplary protocols below. A compound of the invention that inhibits the activity of BRS-3 receptor, e.g. a compound having inverse agonist activity or antagonist activity at BRS-3, can be shown to be a compound having anxiolytic activity (e.g., to be an anxiolytic agent) using the exemplary protocols below. It is expressly contemplated that a drug (e.g., test compound or diazepam) may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat. It is expressly contemplated that a protocol below may alternatively be carried out using a mammal other than a rat. In certain embodiments, the mammal other than a rat is a mouse.

### Protocol I

Animals: Animals used in this study are male B6.129X1-Brs3^{tm1Jfb}/J mice (BRS-3 knockout mice; stock number 004366) and control male C57BL/6J mice (stock number 000664) weighing 20-22 g at the start of the experiment purchased from Jackson Laboratory (Bar Harbor, Me.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

Mice, in experimental (BRS-3 knockout mouse alone and BRS-3 knockout mouse plus diazepam) and control (control mouse alone and control mouse plus diazepam) groups, are tested 30 min after administration of drug. The mouse is placed in the center of the maze facing one of the open arms. The mouse's locomotion is tracked over a 5 min test session using photocells interfaced to a computer. The computer measures the number of entries into each arm and the time spend on each arm.

Drug administration: Diazepam is administered 30 min prior to the test session. Diazepam is administered at, e.g., 0.1 mg/kg, 0.3 mg/kg, 1 mg/kg, 3 mg/kg, 10 mg/kg, 30 mg/kg or 100 mg/kg.

Data Analysis

A greater percentage of time spent on the open arms by mice in the BRS-3 knockout mouse alone group in comparison with the percentage of time spent on the open arms by mice in the control mouse alone group is indicative of a compound that inhibits the activity of BRS-3 receptor, e.g. a compound having inverse agonist activity or antagonist activity at BRS-3, being a compound having anxiolytic activity (e.g., being an anxiolytic agent). A greater percentage of time spent on the open arms by mice in the BRS-3 knockout mouse plus diazepam group in comparison with the percentage of time spent on the open arms by mice in the control mouse plus diazempam group is indicative of a compound that inhibits the activity of BRS-3 receptor, e.g. a compound having inverse agonist activity or antagonist activity at BRS-3, being a compound having anxiolytic activity (e.g., being an anxiolytic agent).

### Protocol II

Animals: Animals used in this study are male Sprague Dawley rats (Harlan, UK) weighing between 280-350 g. The rats are housed two per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Rats are allowed at least three days of habituation to the animal facility before testing.

Procedures:

Rats, in experimental (test compound alone and test compound plus diazepam) and control (vehicle-control and diazepam-control) groups, are tested 30 min after administration of drug. The rat is placed in the center of the maze facing one of the open arms. The rat's locomotion is tracked over a 5 min test session using photocells interfaced to a computer. The computer measures the number of entries into each arm and the time spend on each arm.

Drug administration: All drugs and vehicle are administered 30 min prior to the test session. Drugs (e.g., test compounds and diazepam) are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that inhibits the activity of BRS-3 receptor, e.g. a compound having inverse agonist activity or antagonist activity at BRS-3.

Data Analysis

A greater percentage of time spent on the open arms by rats in the test compound alone group in comparison with the percentage of time spent on the open arms by rats in the vehicle-control group is indicative of the compound administered in the experimental group being a compound having anxiolytic activity (e.g., being an anxiolytic agent). A greater percentage of time spent on the open arms by rats in the test compound plus diazepam group in comparison with the percentage of time spent on the open arms by rats in the diazepam-control group is indicative of the compound administered in the experimental group being a compound having anxiolytic activity (e.g., being an anxiolytic agent). It is expressly contemplated that said test compound which is a compound that inhibits the activity of BRS-3 receptor, e.g. a compound having inverse agonist activity or antagonist activity at BRS-3, can be shown to be a compound having anxiolytic activity (e.g., to be an anxiolytic agent).

### Example 21

### Spatial Water Maze

The spatial water maze has been used extensively as a test of spatial learning and memory. Mice or rats are trained to escape from the water by swimming to a platform that is submerged just below the surface of the water. Since the platform is not visible to the animal, it has to utilize visual extra-maze cues in the area of the tank to locate the platform. Testing by spatial water maze is carried out essentially as described by Collinson et al (J Neurosci (2002) 22:5572-5580) and Dawson et al (J Pharmacol Exp Ther (2006) 316:1335-1345).

A compound of the invention can be shown to be a compound having cognition-enhancing activity (e.g., to be a cognition-enhancing agent) using the exemplary protocols below. A compound of the invention that stimulates the activity of BRS-3 receptor, e.g. a compound having agonist activity or partial agonist activity at BRS-3, can be shown to be a compound having cognition-enhancing activity (e.g., to be a cognition-enhancing agent) using the exemplary protocols below. It is expressly contemplated that a drug (e.g., test compound or diazepam or α5IA) may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat. It is expressly contemplated that a protocol below may alternatively be carried out using a mammal other than a rat. In certain embodiments, the mammal other than a rat is a mouse.

### Protocol I

Animals: Animals used in this study are male B6.129X1-Brs3^{tm1Jfb}/J mice (BRS-3 knockout mice; stock number 004366) and control male C57BL/6J mice (stock number 000664) weighing 20-22 g at the start of the experiment purchased from Jackson Laboratory (Bar Harbor, Me.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

Mice, in experimental (BRS-3 knockout mouse alone and BRS-3 knockout mouse plus diazepam and BRS-3 knockout mouse plus α5IA (3-(5-methylisoxazol-3-yl)-6-[(1-methyl-1,2,3-triazol-4-yl)methyl-oxy]-1,2,4-triazolo[3,4-a]phthalazine; Dawson et al, J Pharmacol Exp Ther (2006) 316:1335-1345)) and control (control mouse alone and control mouse plus diazepam and control mouse plus α5IA) groups, are tested daily in a first trial 30 min after administration of drug. Animals are given four trials daily for 10 days.

Drug administration: Diazepam or α5IA is administered 30 min prior to trial 1. Diazepam or α5IA is administered at, e.g., 0.1 mg/kg, 0.3 mg/kg, 1 mg/kg, 3 mg/kg, 10 mg/kg, 30 mg/kg or 100 mg/kg.

Data Analysis

A savings score is obtained by subtracting the trial 2 latency from the trial 1 latency. A greater savings score for the control mouse alone group in comparison with the savings score for the BRS-3 knockout mouse alone group is indicative of a compound that stimulates the activity of BRS-3 receptor, e.g. a compound having agonist activity or partial agonist activity at BRS-3, being a compound having cognition-enhancing activity (e.g., being a cognition-enhancing agent). A greater savings score for the control mouse plus diazepam group in comparison with the savings score for the BRS-3 knockout mouse plus diazepam group is indicative of a compound that stimulates the activity of BRS-3 receptor, e.g. a compound having agonist activity or partial agonist activity at BRS-3, being a compound having cognition-enhancing activity (e.g., being a cognition-enhancing agent). A greater savings score for the control mouse plus α5A group in comparison with the savings score for the BRS-3 knockout mouse plus α5IA group is indicative of a compound that stimulates the activity of BRS-3 receptor, e.g. a compound having agonist activity or partial agonist activity at BRS-3, being a compound having cognition-enhancing activity (e.g., being a cognition-enhancing agent).

### Protocol II

Animals: Animals used in this study are male hooded Lister rats (Charles River, UK Ltd) weighing between 280-350 g. The rats are housed two per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Rats are allowed at least three days of habituation to the animal facility before testing.

Procedures:

Rats, in experimental (test compound alone and test compound plus diazepam and test compound plus α5IA) and control (vehicle-control and diazepam-control and α5A-control) groups, are tested daily in a first trial 30 min after administration of drug. Animals are given four trials daily for 10 days.

Drug administration: All drugs and vehicle are administered 30 min prior to trial 1. Drugs (e.g., test compounds and diazepam and α5IA) are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that stimulates the activity of BRS-3 receptor, e.g. a compound having agonist activity or partial agonist activity at BRS-3.

Data Analysis

A savings score is obtained by subtracting the trial 2 latency from the trial 1 latency. A greater savings score for the test compound alone group in comparison with the savings score for the vehicle-control group is indicative of the compound administered in the experimental group being a compound having cognition-enhancing activity (e.g., being a cognition-enhancing agent).

A greater savings score for the test compound plus diazepam group in comparison with the savings score for the diazepam-control group is indicative of the compound administered in the experimental group being a compound having cognition-enhancing activity (e.g., being a cognition-enhancing agent). A greater savings score for the test compound plus α5A group in comparison with the savings score for the α5A-control group is indicative of the compound administered in the experimental group being a compound having cognition-enhancing activity (e.g., being a cognition-enhancing agent). It is expressly contemplated that said test compound which is a compound that stimulates the activity of BRS-3 receptor, e.g. a compound having agonist activity or partial agonist activity at BRS-3, can be shown to be a compound having cognition-enhancing activity (e.g., to be an cognition-enhancing agent).

### Example 22

### Rat Polysomnography Protocol for Showing Activity for Promoting Sleep or Promoting Wakefulness by a Compound of the Invention

A compound of the invention can be shown to be a compound having activity for promoting sleep (e.g., to be a sleep-promoting agent) using the exemplary protocol below. A compound of the invention that inhibits the activity of BRS-3 receptor, e.g. a compound having inverse agonist activity or antagonist activity at BRS-3, can be shown to be a compound suitable for promoting sleep (e.g., to be a sleep-promoting agent) using the polysomnography protocol below.

A compound of the invention can be shown to be a compound having activity for promoting wakefulness (e.g., to be a wakefulness-promoting agent) using the exemplary protocol below. A compound of the invention that stimulates the activity of BRS-3 receptor, e.g. a compound having agonist activity or partial agonist activity at BRS-3, can be shown to be a compound suitable for promoting wakefulness (e.g., to be a wakefulness-promoting agent) using the polysomnography protocol below.

It is expressly contemplated that a drug (e.g., test compound or diazepam) may be administered, e.g., orally, intraperitoneally, intracerebroventricularly or intravenously. It is expressly contemplated that a protocol below may alternatively be carried out using a mammal other than a rat. In certain embodiments, the mammal other than a rat is a mouse.

Animals: Male Sprague-Dawley rats (225-350 g) (Harlan, San Diego, Calif.) are singly housed and maintained in a humidity—(30-70%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Rats are allowed at least three days of habituation to the animal facility before surgery.

Procedures:

Rats are anaesthetized with a ketamine/xylazine mixture, and surgically prepared for EEG (electroencephalogram) and electromyogram (EMG) recording. After 2-3 weeks of post-surgical recovery, rats are habituated to polypropylene test cages for at least three days. On test days, the rats are placed in the test chambers and habituated overnight. At 10 am the next day, the rats are administered the test compound plus diazepam or diazepam alone, connected to the recording apparatus, and placed back into the test chambers for 3 h.

Drug administration: Drugs (e.g., test compounds and diazepam) are administered at, e.g., 0.1 mg/kg, 0.3 mg/kg, 1 mg/kg, 3 mg/kg, 10 mg/kg, 30 mg/kg or 100 mg/kg.

Data Analysis

EEG and EMG data are digitized and stored in 10 s epochs over the three hour test period. These data are then visually scored, and each 10 s epoch characterized as either a non-REM sleep, REM sleep, or waking episode. Total sleep time over the three hour period was calculated for each rat after either vehicle administration or test compound. Percent increase in sleep is then derived for each rat.

A compound that increases the duration of sleep in comparison to the diazepam-control is a compound suitable for promoting sleep. A compound that promotes one or more of the reduction of the sleep onset latency period, reduction of the number of nighttime awakenings, and prolongation of the amount of time in delta-wave sleep without affecting REM sleep in comparison to the diazepam control is a compound suitable for promoting sleep. A compound that promotes sleep consolidation in comparison to the diazepam control is a compound suitable for promoting sleep.

A compound that decreases the duration of sleep in comparison to the diazepam-control is a compound suitable for promoting wakefulness. A compound that suppresses one or more of the reduction of the sleep onset latency period, reduction of the number of nighttime awakenings, and prolongation of the amount of time in delta-wave sleep without affecting REM sleep in comparison to the diazepam control is a compound suitable for promoting wakefulness. A compound that suppresses sleep consolidation in comparison to the diazepam control is a compound suitable for promoting wakefulness.

It is expressly contemplated that the duration of the test period may be longer or shorter than 3 h and that the time of compound administration may be other than 10 am. It is also expressly contemplated that the polysomnography may be carried out in a mammal other than a rat. In certain embodiments, the mammal other than a rat is a non-human mammal. In certain embodiments, the mammal other than a rat is a mouse. In certain embodiments, the mammal other than a rat is a human.

### Example 23

### Mouse Polysomnography Protocol for Showing Activity for Promoting Sleep or Promoting Wakefulness by a Compound of the Invention

A compound of the invention can be shown to be a compound having activity for promoting sleep (e.g., to be a sleep-promoting agent) using the exemplary protocol below. A compound of the invention that inhibits the activity of BRS-3 receptor, e.g. a compound having inverse agonist activity or antagonist activity at BRS-3, can be shown to be a compound having sleep-promoting activity (e.g., to be a sleep-promoting agent) using the polysomnography protocol below.

A compound of the invention can be shown to be a compound having activity for promoting wakefulness (e.g., to be a wakefulness-promoting agent) using the exemplary protocol below. A compound of the invention that stimulates the activity of BRS-3 receptor, e.g. a compound having agonist activity or partial agonist activity at BRS-3, can be shown to be a compound having wakefulness-promoting activity (e.g., to be a wakefulness-promoting agent) using the polysomnography protocol below.

It is expressly contemplated that a drug (e.g., diazepam) may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat. It is also expressly contemplated that the duration of the test period may be longer or shorter than 3 h and that the time of compound administration may be other than 10 am.

Animals: Animals used in this study are male B6.129X1-Brs3^{tm1Jfb}/J mice (BRS-3 knockout mice; stock number 004366) and control male C57BL/6J mice (stock number 000664) weighing 20-22 g at the start of the experiment purchased from Jackson Laboratory (Bar Harbor, Me.). The mice are housed four per cage and maintained in a humidity —(40-60%) and temperature—(20-22° C.) controlled facility on a 12 h:12 h light/dark cycle (lights on at 6:30 A.M.) with free access to food (Harlan-Teldad Western Res., Orange, Calif., Rodent Diet 8604) and water. Mice are allowed at least three days of habituation to the animal facility before surgery.

Procedures:

Mice are anaesthetized with a ketamine/xylazine mixture, and surgically prepared for EEG (electroencephalogram) and electromyogram (EMG) recording. After 2-3 weeks of post-surgical recovery, mice are habituated to polypropylene test cages for at least three days. On test days, the mice are placed in the test chambers and habituated overnight. At 10 am the next day, the mice are administered diazepam or vehicle, connected to the recording apparatus, and placed back into the test chambers for 3 h.

Drug administration: Diazepam is administered at, e.g., 0.1 mg/kg, 0.3 mg/kg, 1 mg/kg, 3 mg/kg, 10 mg/kg, 30 mg/kg or 100 mg/kg.

Data Analysis

EEG and EMG data are digitized and stored in 10 s epochs over the three hour test period. These data are then visually scored, and each 10 s epoch characterized as either a non-REM sleep, REM sleep, or waking episode. Total sleep time over the three hour period was calculated for each rat after either vehicle administration or test compound. Percent increase in sleep is then derived for each mouse.

An increase in the duration of sleep in the BRS-3 knockout alone mice in comparison to the control alone mice is indicative of a compound that inhibits the activity of BRS-3 receptor, e.g. a compound having inverse agonist activity or antagonist activity at BRS-3, being a compound having sleep-promoting activity (e.g., being a sleep-promoting agent). Promotion of one or more of the reduction of the sleep onset latency period, reduction of the number of nighttime awakenings, and prolongation of the amount of time in delta-wave sleep without affecting REM sleep observed for the BRS-3 knockout alone mice in comparison to the control alone mice is indicative of a compound that inhibits the activity of BRS-3 receptor, e.g. a compound having inverse agonist activity or antagonist activity at BRS-3, being a compound having sleep-promoting activity (e.g., being a sleep-promoting agent). Promotion of sleep consolidation observed for the BRS-3 knockout alone mice in comparison to the control alone mice is indicative of a compound that inhibits the activity of BRS-3 receptor, e.g. a compound having inverse agonist activity or antagonist activity at BRS-3, being a compound having sleep-promoting activity (e.g., being a sleep-promoting agent).

An increase in the duration of sleep in the BRS-3 knockout plus diazepam mice in comparison to the control plus diazepam mice is indicative of a compound that inhibits the activity of BRS-3 receptor, e.g. a compound having inverse agonist activity or antagonist activity at BRS-3, being a compound having sleep-promoting activity (e.g., being a sleep-promoting agent). Promotion of one or more of the reduction of the sleep onset latency period, reduction of the number of nighttime awakenings, and prolongation of the amount of time in delta-wave sleep without affecting REM sleep observed for the BRS-3 knockout plus diazepam mice in comparison to the control plus diazepam mice is indicative of a compound that inhibits the activity of BRS-3 receptor, e.g. a compound having inverse agonist activity or antagonist activity at BRS-3, being a compound having sleep-promoting activity (e.g., being a sleep-promoting agent). Promotion of sleep consolidation observed for the BRS-3 knockout plus diazepam mice in comparison to the control plus diazepam mice is indicative of a compound that inhibits the activity of BRS-3 receptor, e.g. a compound having inverse agonist activity or antagonist activity at BRS-3, being a compound having sleep-promoting activity (e.g., being a sleep-promoting agent).

An increase in the duration of sleep in the BRS-3 knockout alone mice in comparison to the control alone mice is indicative of a compound that stimulates the activity of BRS-3 receptor, e.g. a compound having agonist activity or partial agonist activity at BRS-3, being a compound having wakefulness-promoting activity (e.g., being a wakefulness-promoting agent). Promotion of one or more of the reduction of the sleep onset latency period, reduction of the number of nighttime awakenings, and prolongation of the amount of time in delta-wave sleep without affecting REM sleep observed for the BRS-3 knockout alone mice in comparison to the control alone mice is indicative of a compound that stimulates the activity of BRS-3 receptor, e.g. a compound having agonist activity or partial agonist activity at BRS-3, being a compound having wakefulness-promoting activity (e.g., being a wakefulness-promoting agent). Promotion of sleep consolidation observed for the BRS-3 knockout alone mice in comparison to the control alone mice is indicative of a compound that stimulates the activity of BRS-3 receptor, e.g. a compound having agonist activity or partial agonist activity at BRS-3, being a compound having wakefulness-promoting activity (e.g., being a wakefulness-promoting agent).

An increase in the duration of sleep in the BRS-3 knockout plus diazepam mice in comparison to the control plus diazepam mice is indicative of a compound that stimulates the activity of BRS-3 receptor, e.g. a compound having agonist activity or partial agonist activity at BRS-3, being a compound having wakefulness-promoting activity (e.g., being a wakefulness-promoting agent). Promotion of one or more of the reduction of the sleep onset latency period, reduction of the number of nighttime awakenings, and prolongation of the amount of time in delta-wave sleep without affecting REM sleep observed for the BRS-3 knockout plus diazepam mice in comparison to the control plus diazepam mice is indicative of a compound that stimulates the activity of BRS-3 receptor, e.g. a compound having agonist activity or partial agonist activity at BRS-3, being a compound having wakefulness-promoting activity (e.g., being a wakefulness-promoting agent). Promotion of sleep consolidation observed for the BRS-3 knockout plus diazepam mice in comparison to the control plus diazepam mice is indicative of a compound that stimulates the activity of BRS-3 receptor, e.g. a compound having agonist activity or partial agonist activity at BRS-3, being a compound having wakefulness-promoting activity (e.g., being a wakefulness-promoting agent).

### Example 24

### Prepulse Inhibition

One of the symptoms of schizophrenia is a decrease in the capacity to filter and process novel sensory or cognitive stimuli. This can be demonstrated in both animals and humans by using a paradigm called prepulse inhibition (PPD. If an intense, sudden stimulus is presented, it elicits a startle response, which can be monitored in animals by measuring the flinching of the animal. If the startling stimulus is preceded by a weak prepulse, then the startle response is inhibited. Schizophrenia patients show a weak or deficient PPI, reflecting a lack of inhibitory function or sensorimotor gating. Prepulse inhibition in animals has been used as a model for screening drugs to treat schizophrenia. If the phenotype of a weak PPI can be reversed in an animal model by a compound, then it is indicative that the compound could be used to treat schizophrenia. Certain strains of mice, such as C57BL/6J naturally display poor PPI. (Ouagazzal et al, Psychopharmacol (2001) 156:273-283).

A compound of the invention can be shown to be a compound having antischizophrenic activity (e.g., to be an antischizophrenic agent) using the exemplary protocol below. A compound of the invention can be shown to be a compound having antipsychotic activity (e.g., to be an antipsychotic agent) using the exemplary protocol below. (See, e.g., Ouagazzal et al, Psychopharmacol (2001) 156:273-283.) A compound of the invention that inhibits the activity of BRS-3 receptor, e.g. a compound having inverse agonist activity or antagonist activity at BRS-3, can be shown to be a compound having antischizophrenic activity (e.g., to be an antischizophrenic agent) using the exemplary protocol below. A compound of the invention that inhibits the activity of BRS-3 receptor, e.g. a compound having inverse agonist activity or antagonist activity at BRS-3, can be shown to be a compound having antipsychotic activity (e.g., to be an antipsychotic agent) using the exemplary protocol below. It is expressly contemplated that a drug (e.g., test compound or diazepam) may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat.

### Protocol

Animals: Animals used in this study are naïve male B6.129X1-Brs3^{tm1Jfb}/J mice (BRS-3 knockout mice; stock number 004366) and control naïve male C57BL/6J mice (stock number 000664) weighing 20-22 g at the start of the experiment (Jackson Laboratory, Bar Harbor, Me.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

Testing is conducted in startle devices (SRLAB, San Diego Instruments, San Diego, Calif.) each consisting of a 5.1 cm (outside diameter) Plexiglas cylinder mounted on a Plexiglas platform in a ventilated, sound-attenuated cubicle with a high frequency loudspeaker (28 cm above the cylinder) producing all acoustic stimuli. The background noise of each chamber is 70 dB. Movements within the cylinder are detected and transduced by a piezoelectric accelerometer attached to the Plexiglas base, digitized and stored by a computer. Beginning at the stimulus onset, 65 readings of 1 ms duration are recorded to obtain the animal's startle amplitude.

Twelve naïve mice per group (BRS-3 knockout mouse alone group, BRS-3 knockout mouse plus diazepam group, control mouse alone group, control mouse plus diazepam group, control mouse plus test compound group, control mouse plus test compound plus diazepam group) are tested. Each session is initiated with a 5-min acclimation period followed by five successive 110 dB trials. These trials are not included in the analysis. Six different trial types are then presented: startle pulse (ST110, 110 dB/40 ms), low prepulse stimulus given alone (P74, 74 dB/20 ms), high prepulse stimulus given alone (P90, 90 dB/20 ms), P74 or P90 given 100 ms before the onset of the startle pulse (PP74 and PP90, respectively), and finally a trial where only the background noise is presented (NST) in order to measure the baseline movement in the cylinders. All trials are applied 10 times and presented in random order (P74 and P90 are only given 5 times) and the average inter-trial interval (TTI) is 15 s (10-20 s).

Drug administration: Drug (test compound, diazepam, test compound plus diazepam) is administered 30 min prior to the test session. Test compound and diazepam are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that inhibits the activity of BRS-3 receptor, e.g. a compound having inverse agonist activity or antagonist activity at BRS-3.

Data Analysis

Greater percentage prepulse inhibition in the BRS-3 knockout mouse alone group in comparison with the control mouse alone group is indicative of a compound that inhibits the activity of BRS-3 receptor, e.g. a compound having inverse agonist activity or antagonist activity at BRS-3, being a compound having antischizophrenic activity (e.g., being an antischizophrenic agent). Greater percentage prepulse inhibition in the BRS-3 knockout mouse plus diazepam group in comparison with the control mouse plus diazepam group is indicative of a compound that inhibits the activity of BRS-3 receptor, e.g. a compound having inverse agonist activity or antagonist activity at BRS-3, being a compound having antischizophrenic activity (e.g., being an antischizophrenic agent). Greater percentage prepulse inhibition in the control mouse plus test compound group in comparison with the control mouse alone group is indicative of the compound administered in the experimental group being a compound having antischizophrenic activity (e.g., being an antischizophrenic agent). Greater percentage prepulse inhibition in the control mouse plus test compound plus diazepam group in comparison with the control mouse plus diazepam group is indicative of the compound administered in the experimental group being a compound having antischizophrenic activity (e.g., being an antischizophrenic agent). It is expressly contemplated that said test compound which is a compound that inhibits the activity of BRS-3 receptor, e.g. a compound having inverse agonist activity or antagonist activity at BRS-3, can be shown to be a compound having antischizophrenic activity (e.g., to be an antischizophrenic agent).

Greater percentage prepulse inhibition in the BRS-3 knockout mouse alone group in comparison with the control mouse alone group is indicative of a compound that inhibits the activity of BRS-3 receptor, e.g. a compound having inverse agonist activity or antagonist activity at BRS-3, being a compound having antipsychotic activity (e.g., being an antipsychotic agent). Greater percentage prepulse inhibition in the BRS-3 knockout mouse plus diazepam group in comparison with the control mouse plus diazepam group is indicative of a compound that inhibits the activity of BRS-3 receptor, e.g. a compound having inverse agonist activity or antagonist activity at BRS-3, being a compound having antipsychotic activity (e.g., being an antipsychotic agent). Greater percentage prepulse inhibition in the control mouse plus test compound group in comparison with the control mouse alone group is indicative of the compound administered in the experimental group being a compound having antipsychotic activity (e.g., being an antipsychotic agent). Greater percentage prepulse inhibition in the control mouse plus test compound plus diazepam group in comparison with the control mouse plus diazepam group is indicative of the compound administered in the experimental group being a compound having antipsychotic activity (e.g., being an antipsychotic agent). It is expressly contemplated that said test compound which is a compound that inhibits the activity of BRS-3 receptor, e.g. a compound having inverse agonist activity or antagonist activity at BRS-3, can be shown to be a compound having antipsychotic activity (e.g., to be an antipsychotic agent).

### Example 25

### Object Recognition Task

The object recognition task, originally developed by Ennaceur and Delacour in rats (Behavioural Brain Research, 31, 47-59, 1988), relies on the innate tendency of rodents to explore novel objects in their environment and can be used to determine whether an animal remembers a previously encountered object. Memory for a previously encountered (familiar) object can be measured by the relative time spent exploring this object compared to the time spent exploring a novel object. Novel objects are normally explored more than familiar ones. Therefore, if an animal remembers an object, the object should be explored less than a novel object. If the familiar object has been forgotten, it will be explored as much as a novel object. Memory can be modulated by adjusting the retention interval between the sample presentation and the test session during which a novel object is presented. In order to evaluate the cognitive enhancing properties of compounds, a 24-hour retention interval can be used. Under this condition, control mice fail to show a preference for, while mice injected with a cognitive-enhancing compound may spend more time exploring the novel object. A shorter retention interval may be used to examine compounds that may impair cognitive processing. (See, e.g., Nilsson et al, Neurosci (2007) 149:123-130.)

A compound of the invention can be shown to be a compound having cognition-enhancing activity (e.g., to be a cognition-enhancing agent) using the exemplary protocols below. A compound of the invention that stimulates the activity of BRS-3 receptor, e.g. a compound having agonist activity or partial agonist activity at BRS-3, can be shown to be a compound having cognition-enhancing activity (e.g., to be a cognition-enhancing agent) using the exemplary protocols below. It is expressly contemplated that a drug (e.g., test compound or diazepam or α5IA) may be administered, e.g., orally, intraperitoneally, intracerebroventricularly or intravenously. It is expressly contemplated that a protocol below may alternatively be carried out using a mammal other than a mouse. In certain embodiments, the mammal other than a mouse is a rat.

### Protocol I

Animals: Animals used in this study are male B6.129X1-Brs3^{tm1Jfb}/J mice (BRS-3 knockout mice; stock number 004366) and control male C57BL/6J mice (stock number 000664) weighing 20-22 g at the start of the experiment purchased from Jackson Laboratory (Bar Harbor, Me.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

The testing arena is an open-field of dark gray, matte finished ABS plastic (65×45×45 (H) cm) with a magnetic strip placed under a plastic insert at the base. The objects are small pyramids (9.5 cm long, 5.7 cm high) made of either red plastic (Lego) or gray metal copies. All objects have a thin magnetic strip on their bases to attach to the open field. The arena is illuminated by a ceiling light of approximately 25 lux at the test floor level. A CCD video camera is placed above the open-field and is connected to a monitor and VCR in the adjacent room. The number of times and length of time mice spend exploring familiar (F) and novel (N) objects is recorded manually by the experimenter.

Groups: BRS-3 knockout mouse alone, BRS-3 knockout mouse plus diazepam, BRS-3 knockout mouse plus α5IA, control mouse alone, control mouse plus diazepam, and control mouse plus α5A.

Drug administration: Drugs (e.g., diazepam and α5IA) are administered at, e.g., 0.1 mg/kg, 0.3 mg/kg, 1 mg/kg, 3 mg/kg, 10 mg/kg, 30 mg/kg or 100 mg/kg.

Pre-exposure: The first day animals explore the open-field for a 5-min habituation session.

Training day: On the following day, two identical objects are presented at opposite corners of the open-field (10 cm from walls) for another 5 min session.

Immediately after training, mice are injected with drug or vehicle. Mice are brought back, e.g., 1 h, 4 h, 12 h or 24 h later for testing.

Test: One of the objects previously presented is replaced by a new object, and animals are again allowed to explore these two objects for another 5 min session. Within a group, the corner where the new object is placed is alternated between animals. Objects are thoroughly washed with 70% ethanol to prevent the animal from using olfactory cues.

Scoring criteria: The basic measurement is the exploration time (sec) of the novel and familiar objects. Exploration is considered as touching or directing the nose to the objects at a distance of 1 cm or less. The difference between two exploration times on the new (N) and familiar (F) objects is taken as the discrimination index (d=N−F). In order to show that recognition scores are not biased by differences in overall levels of exploration, another discrimination index can be used (D=N−F/N+F).

A greater discrimination index for the control mouse alone group in comparison with the discrimination index for the BRS-3 knockout mouse alone group is indicative of a compound that stimulates the activity of BRS-3 receptor, e.g. a compound having agonist activity or partial agonist activity at BRS-3, being a compound having cognition-enhancing activity (e.g., being a cognition-enhancing agent). A greater discrimination index for the control mouse plus diazepam group in comparison with the discrimination index for the BRS-3 knockout mouse plus diazepam group is indicative of a compound that stimulates the activity of BRS-3 receptor, e.g. a compound having agonist activity or partial agonist activity at BRS-3, being a compound having cognition-enhancing activity (e.g., being a cognition-enhancing agent). A greater discrimination index for the control mouse plus α5IA group in comparison with the discrimination index for the BRS-3 knockout mouse plus α5A group is indicative of a compound that stimulates the activity of BRS-3 receptor, e.g. a compound having agonist activity or partial agonist activity at BRS-3, being a compound having cognition-enhancing activity (e.g., being a cognition-enhancing agent).

### Protocol II

Animals: Animals used in this study are male C57BL/6J mice weighing 20-22 g at the start of the experiment (Jackson Laboratory, Bar Harbor, Me.). The mice are housed four per cage and maintained in a humidity—(40-60%) and temperature—(20-22° C.) controlled facility on a 12 h:12 h light/dark cycle (lights on at 6:30 A.M.) with free access to food (Harlan-Teklad Western Res., Orange, Calif., Rodent Diet 8604) and water. Mice are allowed at least three days of habituation to the animal facility before testing.

Procedures:

The testing arena is an open-field of dark gray, matte finished ABS plastic (65×45×45 (H) cm) with a magnetic strip placed under a plastic insert at the base. The objects are small pyramids (9.5 cm long, 5.7 cm high) made of either red plastic (Lego) or gray metal copies. All objects have a thin magnetic strip on their bases to attach to the open field. The arena is illuminated by a ceiling light of approximately 25 lux at the test floor level. A CCD video camera is placed above the open-field and is connected to a monitor and VCR in the adjacent room. The number of times and length of time mice spend exploring familiar (F) and novel (N) objects is recorded manually by the experimenter.

Groups: Test compound, test compound plus diazepam, test compound plus α5IA, vehicle-control, diazepam-control, α5IA-control.

Drug administration: Drugs (e.g., test compounds and diazepam and α5IA) are administered at, e.g., 0.1 mg/kg, 0.3 mg/kg, 1 mg/kg, 3 mg/kg, 10 mg/kg, 30 mg/kg or 100 mg/kg. It is expressly contemplated that said test compound is a compound that stimulates the activity of BRS-3 receptor, e.g. a compound having agonist activity or partial agonist activity at BRS-3.

Pre-exposure: The first day animals explore the open-field for a 5-min habituation session.

Training day: On the following'day, two identical objects are presented at opposite corners of the open-field (10 cm from walls) for another 5 min session.

Immediately after training, mice are injected with drug or vehicle. Mice are brought back, e.g., 1 h, 4 h, 12 h or 24 h later for testing.

Test: One of the objects previously presented is replaced by a new object, and animals are again allowed to explore these two objects for another 5 min session. Within a group, the corner where the new object is placed is alternated between animals. Objects are thoroughly washed with 70% ethanol to prevent the animal from using olfactory cues.

Scoring criteria: The basic measurement is the exploration time (sec) of the novel and familiar objects. Exploration is considered as touching or directing the nose to the objects at a distance of 1 cm or less. The difference between two exploration times on the new (N) and familiar (F) objects is taken as the discrimination index (d=N−F). In order to show that recognition scores are not biased by differences in overall levels of exploration, another discrimination index can be used (D=N−F/N+F).

A greater discrimination index for the test compound alone group in comparison with the discrimination index for the vehicle-control group is indicative of the compound administered in the experimental group being a compound having cognition-enhancing activity (e.g., being a cognition-enhancing agent). A greater discrimination index for the test compound plus diazepam group in comparison with the discrimination index for the diazepam-control group is indicative of the compound administered in the experimental group being a compound having cognition-enhancing activity (e.g., being a cognition-enhancing agent). A greater discrimination index for the test compound plus α5IA group in comparison with the discrimination index for the α5IA-control group is indicative of the compound administered in the experimental group being a compound having cognition-enhancing activity (e.g., being a cognition-enhancing agent). It is expressly contemplated that said test compound which is a compound that stimulates the activity of BRS-3 receptor, e.g. a compound having agonist activity or partial agonist activity at BRS-3, can be shown to be a compound having cognition-enhancing activity (e.g., to be an cognition-enhancing agent).

1.-20. (canceled)

21. A method of promoting sleep or of preventing or treating a sleep disorder ameliorated by promoting sleep or for preventing or treating a GABA-related neurological disorder selected from the group consisting of a sleep disorder ameliorated by promoting sleep, an anxiety disorder, a convulsive disorder, migraine, a depressive disorder, and a psychotic disorder comprising administering to a mammal in need thereof a therapeutically effective amount of an inverse agonist or antagonist of the mammalian BRS-3 or of a pharmaceutically acceptable composition comprising the inverse agonist or antagonist and a pharmaceutically acceptable carrier.

22. A method in accordance with claim 21, wherein the sleep disorder comprises fragmented sleep architecture.

23. A method in accordance with claim 21, wherein said method of promoting sleep or of preventing or treating a sleep disorder ameliorated by promoting sleep comprises promoting sleep consolidation.

24. A method in accordance with claim 21, wherein said method of promoting sleep or of preventing or treating a sleep disorder ameliorated by promoting sleep comprises increasing delta power.

25. A method in accordance with claim 21, wherein the sleep disorder is selected from the group consisting of psychophysiological insomnia, sleep state misperception, idiopathic insomnia, obstructive sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder

26. A method in accordance with claim 21, wherein the sleep disorder is insomnia or wherein the GABA-related neurological disorder is selected from the group consisting of Insomnia, Generalized Anxiety Disorder, Panic Attack, Epilepsy, Migraine, Major Depressive Disorder, and Schizophrenia.

27. A method in accordance with any one of claims 21 to 26, wherein the inverse agonist or antagonist of the mammalian BRS-3 is a BRS-3 selective inverse agonist or antagonist.

28. A method in accordance with any one of claims 21 to 27, wherein the mammal is a human.

29.-66. (canceled)

67. A method of promoting wakefulness or of preventing or treating excessive sleepiness or for preventing or treating a GABA-related neurological disorder selected from the group consisting of a sleep disorder ameliorated by promoting wakefulness and a cognitive disorder comprising administering to a mammal in need thereof a therapeutically effective amount of an agonist or a partial agonist of the mammalian BRS-3 or of a pharmaceutically acceptable composition comprising the agonist or partial agonist and a pharmaceutically acceptable carrier.

68. A method in accordance with claim 67, wherein the excessive sleepiness is associated with a sleep disorder.

69. A method in accordance with claim 68, wherein the sleep disorder is selected from the group consisting of sleep state misperception, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, restless legs syndrome, hypnotic-dependent sleep disorder, toxin-induced sleep disorder, time zone change (jet lag) syndrome, shift work sleep disorder, irregular sleep-wake pattern, delayed sleep phase syndrome, advanced sleep phase syndrome, and non-24 hour sleep-wake disorder.

70. A method in accordance with claim 68, wherein the sleep disorder is narcolepsy or wherein the GABA-related neurological disorder is selected from the group consisting of Narcolepsy, Dementia, and Dementia of the Alzheimer's Type.

71. A method in accordance with claim 67, wherein the excessive sleepiness is associated with a neurological disorder.

72. A method in accordance with claim 67, wherein the excessive sleepiness is associated with a psychiatric disorder.

73. A method in accordance with any one of claims 67 to 72, wherein the agonist or partial agonist of the mammalian BRS-3 is a BRS-3 selective agonist or partial agonist.

74. A method in accordance with any one of claims 67 to 72, wherein the mammal is a human.

75.-90. (canceled)

91. A method of screening candidate compounds for a pharmaceutical agent for a GABA-related neurological disorder selected from the group consisting of a sleep disorder ameliorated by promoting sleep, an anxiety disorder, a convulsive disorder, migraine, a depressive disorder, a psychotic disorder, a sleep disorder ameliorated by promoting wakefulness, and a cognitive disorder, said method comprising the elements:
(a) providing a host cell or a membrane of a host cell that comprises a G protein-coupled receptor, said G protein-coupled receptor comprising an amino acid sequence having at least about 75% identity, at least about 80% identity, at least about 85% identity, at least about 90% identity or at least about 95% identity to SEQ ID NO: 2; and
(b) screening candidate compounds against said G protein-coupled receptor.

92. The method of claim 91, wherein the method comprises identifying an agonist of the G protein-coupled receptor.

93. The method of claim 91 or claim 92, wherein the method comprises identifying a partial agonist of the G protein-coupled receptor.

94. The method of claim 91, wherein the method comprises identifying an inverse agonist of the G protein-coupled receptor.

95. The method of claim 91, wherein the method comprises identifying an antagonist of the G protein-coupled receptor.

96. The method of claims 92, 94, or 95, wherein said screening comprises determining whether said agonist, partial agonist, inverse agonist or antagonist promotes sleep, has anxiolytic activity, has anticonvulsant activity, has anti-migraine activity, has antidepressant activity, has antipsychotic activity, promotes wakefulness, or has cognition-enhancing activity.

97. The method of claims 92 94, or 95, wherein the method further comprises formulating said agonist, partial agonist, inverse agonist or antagonist as a pharmaceutical.

98. The method of any claims 91, 92, 94, or 95, wherein the G protein-coupled receptor comprises an amino acid sequence having at least about 95% identity to SEQ ID NO: 2.

99. The method of claims 91, 92, 94, or 95, wherein the G protein-coupled receptor comprises the amino acid sequence of SEQ ID NO: 2.

100.-106. (canceled)


SEQUENCE LISTING (6 sequences)

SEQ ID NO: 1 - DNA, 1200 bp, Homo sapien
atggctcaaaggcagcctcactcacctaatcagactttaatttcaatcacaaatgacacagaatcatcaagctctgtggtttctaacgataacacaaataaaggatggagcggggacaactctccaggaatagaagcattgtgtgccatctatattacttatgctgtgatcatttcagtgggcatccttggaaatgctattctcatcaaagtctttttcaagaccaaatccatgcaaacagttccaaatattttcatcaccagcctggcttttggagatcttttacttctgctaacttgtgtgccagtggatgcaactcactaccttgcagaaggatggctgttcggaagaattggttgtaaggtgctctctttcatccggctcacttctgttggtgtgtcagtgttcacattaacaattctcagcgctgacagatacaaggcagttgtgaagccacttgagcgacagccctccaatgccatcctgaagacttgtgtaaaagctggctgcgtctggatcgtgtctatgatatttgctctacctgaggctatattttcaaatgtatacacttttcgagatcccaataaaaatatgacatttgaatcatgtacctcttatcctgtctctaagaagctcttgcaagaaatacattctctgctgtgcttcttagtgttctacattattccactctctattatctctgtctactattccttgattgctaggaccctttacaaaagcaccctgaacatacctactgaggaacaaagccatgcccgtaagcagattgaatcccgaaagagaattgccagaacggtattggtgttggtggctctgtttgccctctgctggttgccaaatcacctcctgtacctctaccattcattcacttctcaaacctatgtagacccctctgccatgcatttcattttcaccattttctctcgggttttggctttcagcaattcttgcgtaaacccctttgctctctactggctgagcaaaagcttccagaagcattttaaagctcagttgttctgttgcaaggcggagcggcctgagcctcctgttgctgacacctctcttaccaccctggctgtgatgggaacggtcccgggcactgggagcatacagatgtctgaaattagtgtgacctcgttcactgggtgtagtgtgaagcaggcagaggacagattctag

SEQ ID NO: 2 - protein, 399 aa, homo sapien
MAQRQPHSPNQTLISITNDTESSSSVVSNDNTNKGWSGDNSPGIEALCAIYITYAVIISVGILGNAILIKVFFKTKSMQTVPNIFITSLAFGDLLLLLTCVPVDATHYLAEGWLFGRIGCKVLSFIRLTSVGVSVFTLTILSADRYKAVVKPLERQPSNAILKTCVKAGCVWIVSMIFALPEAIFSNVYTFRDPNKNMTFESCTSYPVSKKLLQEIHSLLCFLVFYIIPLSIISVYYSLIARTLYKSTLNIPTEEQSHARKQIESRKRIARTVLVLVALFALCWLPNHLLYLYHSFTSQTYVDPSAMHFIFTIFSRVLAFSNSCVNPFALYWLSKSFQKHFKAQLFCCKAERPEPPVADTSLTTLAVMGTVPGTGSIQMSEISVTSFTGCSVKQAEDRF

SEQ ID NO: 3 - DNA, 30 bp, Artificial
  Primer
acagaattcagaagaaatggctcaaaggca

SEQ ID NO: 4 - DNA, 30 bp, Artificial
  Primer
catggatccttgaaaagctagaatctgtcc

SEQ ID NO: 5 - DNA, 24 bp, Artificial
  Primer
tcccgctctcgattatctctgtct

SEQ ID NO: 6 - DNA, 24 bp, Artificial
  Primer
tcctctcccttcttggcactactg